(19) United States
(12) Patent Application Publication
(10) Pub. No.: US 2023/0348865 A1
(43) Pub. Date: Nov. 2, 2023

(54) ENGINEERED ENZYMES AND METHODS OF MAKING AND USING

(71) Applicant: Genomatica, Inc., San Diego, CA (US)

(72) Inventors: Amit Mahendra SHAH, San Diego, CA (US); Deqiang ZHANG, San Diego, CA (US); Joseph Roy WARNER, Oceanside, CA (US); Benjamin Matthew GRIFFIN, San Diego, CA (US); Russell Scott KOMOR, San Diego, CA (US); Harish NAGARAJAN, San Diego, CA (US); Sankha GHATAK, San Diego, CA (US); Toby Howard RICHARDSON, San Diego, CA (US)

(21) Appl. No.: 17/996,921

(22) PCT Filed: Apr. 23, 2021
(86) PCT No.: PCT/US2021/028768

§ 371 Date: Oct. 21, 2022

Related U.S. Application Data
(60) Provisional application No. 63/015,428, filed on Apr. 24, 2020.

(51) Int. Cl. C12N 9/02; C12P 13/00; C12P 7/04; C12N 15/52; C12N 9/10; C12N 9/88

(52) U.S. Cl. CPC C12N 9/0008; C12Y 102/0103; C12P 13/001; C12P 7/04; C12N 15/52; C12N 9/1029; C12Y 203/01174; C12Y 102/01003; C12N 9/88; C12Y 402/01017; C12N 9/1096

(57) ABSTRACT

The invention provides an engineered carboxylic acid reductase (CAR) enzyme, a nucleic acid encoding the CAR enzyme, and a non-naturally occurring microbial organism comprising an exogenous nucleic acid encoding the CAR, an engineered transaminase (TA) enzyme, and/or a hexamethylenediamine (HMD) transaminase (TA2) enzyme. The invention provides a non-naturally occurring microbial organism that has a 1,6-hexanediol (HDO) pathway with a HDO pathway enzyme expressed in sufficient amounts to produce 6 aminocaproate semi aldehyde, HDO, or both. The invention further provides a non-naturally occurring microbial organism that has an HMD pathway with a HMD pathway enzyme expressed in sufficient amounts to produce 6-aminocaproate semialdehyde, HMD, or both. The invention additionally provides bioderived HMD, 6-aminocaproate semialdehyde, and/or HDO and methods for producing bioderived HMD, 6-aminocaproate semialdehyde, and/or HDO.

### CROSS-REFERENCE TO RELATED APPLICATIONS

This application claims the benefit of U.S. Provisional Patent Application No. 63/015,428, filed Apr. 24, 2020, which is incorporated by reference herein in its entirety.

### SEQUENCE LISTING

This application incorporates by reference a Sequence Listing submitted with this application as an ASCII text file, entitled 12956-492-228_SEQ_LISTING.txt created on Apr. 20, 2021 and having a size of 1,773,544 bytes.

### BACKGROUND

Nylons are polyamides that can be synthesized by the condensation polymerization of a diamine with a dicarboxylic acid or the condensation polymerization of lactams. Nylon 6,6 is produced by reaction of hexamethylenediamine (HMD) and adipic acid, while nylon 6 is produced by a ring opening polymerization of caprolactam. Therefore, adipic acid, hexamethylenediamine, and caprolactam are important intermediates in nylon production.

Microorganisms have been engineered to produce some of the nylon intermediates. However, engineered microorganisms can produce undesirable byproducts as a result of undesired enzymatic activity on pathway intermediates and final products. Such byproducts and impurities therefore increase cost and complexity of biosynthesizing compounds and can decrease efficiency or yield of the desired products.

### SUMMARY

The invention provides an engineered carboxylic acid reductase (CAR) enzyme capable of (a) forming 6-aminocaproate semialdehyde from a 6-aminocaproic acid substrate, (b) forming 6-aminocaproate semialdehyde from a 6-aminocaproic acid substrate at a greater rate as compared to the wild type CAR, (c) having a higher affinity for a 6-aminocaproic acid substrate as compared to the wild type CAR, or any combination of (a), (b), and (c). The engineered CAR enzyme can comprise one or more amino acid alterations at one or more residue positions disclosed herein, for example at least one alteration of an amino acid of SEQ ID NO: 152, 153, or 254. Provided herein is an engineered CAR that has an activity that is at least 20% higher than the activity of the CAR of SEQ ID NO:152, 153 or 254.

The invention also provides the nucleic acid encoding the engineered CAR disclosed herein, which can be operatively linked to a promoter and can be in a vector.

The invention further provides a non-naturally occurring microbial organism comprising an exogenous nucleic acid encoding an engineered CAR disclosed herein, a CAR having an amino acid sequence having at least 50% sequence identity to a CAR disclosed herein, or hexamethylenediamine (TIMID) transaminase (TA2) enzyme having at least 50% sequence identity to a TA2 disclosed herein. In some aspects, the non-naturally occurring microbial organism further contains an exogenous nucleic acid encoding an engineered transaminase (TA) enzyme having a sequence disclosed herein, for example, a TA enzyme having one or more amino acid alterations at one or more positions selected from residues disclosed herein or having at least 50% sequence identity to at least 25 or more contiguous amino acids of any TA sequence disclosed herein. The exogenous nucleic acid can be heterologous or homolgous. The non-naturally occurring microbial organism can comprise a CAR variant, a TA2 variant, and/or a TA variant disclosed herein.

The invention provides a non-naturally occurring microbial organism of any one of claims comprising a hexamethylenediamine (HMD) pathway having a HMD pathway enzyme expressed in sufficient amounts to produce HMD. The HMD pathway comprises (1) 3-oxoadipyl-CoA thiolase, (2) hydroxyadipyl-CoA dehydrogenase (HBD), (3) crotonase, (4) trans-enoylCoA reductase (TER), (5) 6ACA-aldehyde dehydrogenase (6ACA-ALD), (6) 6ACA-transaminase (TA), (7) carboxylic acid reductase (CAR), and (8) HMD-transaminase (TA2). The non-naturally occurring microbial organism can further comprising one or more exogenous nucleic acids encoding a phosphopantetheinyl transferase HMD pathway enzyme. The exogenous nucleic acid can be a heterologous nucleic acid. In some aspects, the non-naturally occurring microbial organism is in a substantially anaerobic culture medium. The microbial organism can be a species of bacteria, yeast, or fungus. In some aspects, the non-naturally occurring microbial organism is capable of producing at least 10% more 6-aminocaproate semialdehyde, HMD or both compared to a control microbial organism that does not contain the exogenous nucleic acid. Further provided herein is a non-naturally occurring microbial organism that converts more: (a) adipate semialdehyde to 6-aminocaproic acid; (b) 6-aminocaproic acid to 6-aminocaproate semialdehyde, and/or (c) 6-aminocaproate semialdehyde to HMD, compared to a control microbial organism that does not contain the exogenous nucleic acid.

Provided herein is a non-naturally occurring microbial organism having an exogenous nucleic acid encoding an engineered CAR disclosed herein, or a CAR comprising an amino acid sequence having at least 50% sequence identity to at least 25 or more contiguous amino acids of the sequence of a CAR disclosed herein. The non-naturally occurring microbial organism can further contain an exogenous nucleic acid encoding (a) an engineered transaminase (TA) enzyme comprising at least one alteration of an amino acid of SEQ ID NOS:1, 13 or 31; (b) an engineered TA enzyme comprising one or more amino acid alterations at one or more positions selected from residues disclosed herein; (c) an engineered TA enzyme comprising at least one amino acid alteration of the engineered protein selected from an alteration disclosed herein and combinations thereof of SEQ ID NO:1, or (d) a transaminase comprising an amino acid sequence having at least 50% sequence identity to at least 25 or more contiguous amino acids of a sequence disclosed herein. The exogenous nucleic acid can be heterologous or homolgous. The non-naturally occurring microbial organism can contain a CAR having an amino acid sequence selected from the group consisting of CAR variants disclosed herein, and/or an engineered TA having an amino acid sequence of a TA variants disclosed herein. In some aspects, the non-naturally occurring microbial organism comprises a 1,6-hexanediol (HDO) pathway having a HDO pathway enzyme expressed in sufficient amounts to produce HDO, wherein said HDO pathway comprises (1) thiolase, (2) hydroxyadipyl-CoA dehydrogenase (HBD), (3) crotonase, (4) trans-enoylCoA reductase (TER), (5) 6ACA-aldehyde dehydrogenase (6ACA-ALD), (6) 6ACA-transaminase (TA), (7) carboxylic acid reductase (CAR), (8) 6-aminocaproate semialdehyde reductase, (9) 6-aminohexanol aminotransferase or oxidoreductase, and (10) 6-hydroxyhexanal reductase. The non-naturally occurring microbial organism can further contain one or more exogenous nucleic acids encoding a phosphopantetheinyl transferase HDO pathway enzyme. The exogenous nucleic acid can be a heterologous nucleic acid. In some aspects, the non-naturally occurring microbial organism is in a substantially anaerobic culture medium. The microbial organism can be a species of bacteria, yeast, or fungus. In some aspects, the non-naturally occurring microbial organism is capable of producing at least 10% more 6-aminocaproate semialdehyde, HDO or both compared to a control microbial organism that does not comprise the exogenous nucleic acid disclosed herein. The non-naturally occurring microbial organism provided herein converts more: (a) adipate semialdehyde to 6-aminocaproic acid, and/or (b) 6-aminocaproic acid to 6-aminocaproate semialdehyde compared to a control microbial organism that does not comprise the exogenous nucleic acid disclosed herein.

The invention further provides a method for producing hexamethylenediamine (HMD), comprising culturing the non-naturally occurring microbial organism disclosed herein under conditions and for a sufficient period of time to produce HMD. The invention also provides a method for producing 1,6-hexanediol (HDO), comprising culturing the non-naturally occurring microbial organism disclosed herein under conditions and for a sufficient period of time to produce HDO. In some aspects, the method further comprises separating the HMD or HDO from other components in the culture. The separating can comprise extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

Provided herein is culture medium comprising bioderived HMD, 6-aminocaproate semialdehyde, and/or HDO that has a carbon-12, carbon-13 and carbon-14 isotope ratio that reflects an atmospheric carbon dioxide uptake source. In some aspects, the bioderived HMD, 6-aminocaproate semialdehyde, and/or HDO is produced by a non-naturally occurring microbial organism disclosed herein or a method disclosed herein. In some aspects, the culture medium comprises the engineered CAR, engineered TA enzyme, the engineered hexamethylenediamine (HMD) transaminase (TA2) enzyme, and/or the aldehyde dehydrogenase (ALD) enzyme disclosed herein. In some aspects, the culture medium contains a nucleic acid encoding the engineered CAR, engineered TA enzyme, the engineered TA2 enzyme, and/or the aldehyde dehydrogenase (ALD) enzyme disclosed herein. In some aspects, the culture medium contains a non-naturally occurring microbial organism disclosed herein. The culture medium can be separated from the non-naturally occurring microbial organism that produces bioderived HMD, 6-aminocaproate semialdehyde, and/or HDO.

Further provided herein is bioderived HMD, 6-aminocaproate semialdehyde, and/or HDO having a carbon-12, carbon-13 and carbon-14 isotope ratio that reflects an atmospheric carbon dioxide uptake source. In some aspects, the bioderived HMD, 6-aminocaproate semialdehyde, and/or HDO is produced by a non-naturally occurring microbial organism and/or a method disclosed herein. In some aspects, the bioderived HMD, 6-aminocaproate semialdehyde, and/or HDO has an Fm value of at least 80%, at least 85%, at least 90%, at least 95% or at least 98%. The invention also provides compositions contain the bioderived HMD, 6-aminocaproate semialdehyde, and/or HDO disclosed herein, and a compound other than the bioderived HMD, 6-aminocaproate semialdehyde, and/or HDO. The composition can contain a portion of the non-naturally occurring microbial organism disclosed herein or a cell lysate or culture supernatant.

### BRIEF DESCRIPTION OF THE DRAWINGS

FIG. 1 shows exemplary pathways from succinyl-CoA and acetyl-CoA to 6-aminocaproate, hexamethylenediamine (HMD), and caprolactam. The enzymes are designated as follows: A) 3-oxoadipyl-CoA thiolase, B) 3-oxoadipyl-CoA reductase, C) 3-hydroxyadipyl-CoA dehydratase, D) 5-carboxy-2-pentenoyl-CoA reductase, E) 3-oxoadipyl-CoA/acyl-CoA transferase, F) 3-oxoadipyl-CoA synthase, G) 3-oxoadipyl-CoA hydrolase, H) 3-oxoadipate reductase, I) 3-hydroxyadipate dehydratase, J) 5-carboxy-2-pentenoate reductase, K) adipyl-CoA/acyl-CoA transferase, L) adipyl-CoA synthase, M) adipyl-CoA hydrolase, N) adipyl-CoA reductase (aldehyde forming), O) 6-aminocaproate transaminase, P) 6-aminocaproate dehydrogenase, Q) 6-aminocaproyl-CoA/acyl-CoA transferase, R) 6-aminocaproyl-CoA synthase, S) amidohydrolase, T) spontaneous cyclization, U) 6-aminocaproyl-CoA reductase (aldehyde forming), V) HMD transaminase, W) HMD dehydrogenase, X) adipate reductase, Y) adipate kinase, Z) adipylphosphate reductase.

FIG. 2 is a graphical representation of the amino acid positions mutated in SEQ ID NO: 1.

FIG. 3 is a graphical representation of the activity of the variants relative to the wild-type SEQ ID NO: 1 control (SEQ ID NO: 1).

FIG. 4 shows an exemplary pathway for synthesis of 6-amino caproic acid and adipate using lysine as a starting point.

FIG. 5 shows an exemplary caprolactam synthesis pathway using adipyl-CoA as a starting point.

FIG. 6 shows exemplary pathways to 6-aminocaproate from pyruvate and succinic semialdehyde. Enzymes are A) HODH aldolase, B) OHED hydratase, C) OHED reductase, D) 2-OHD decarboxylase, E) adipate semialdehyde aminotransferase and/or adipate semialdehyde oxidoreductase (aminating), F) OHED decarboxylase, G) 6-OHE reductase, H) 2-OHD aminotransferase and/or 2-OHD oxidoreductase (aminating), I) 2-AHD decarboxylase, J) OHED aminotransferase and/or OHED oxidoreductase (aminating), K) 2-AHE reductase, L) HODH formate-lyase and/or HODH dehydrogenase, M) 3-hydroxyadipyl-CoA dehydratase, N) 2,3-dehydroadipyl-CoA reductase, O) adipyl-CoA dehydrogenase, P) OHED formate-lyase and/or OHED dehydrogenase, Q) 2-OHD formate-lyase and/or 2-OHD dehydrogenase. Abbreviations are: HODH=4-hydroxy-2-oxoheptane-1,7-dioate, OHED=2-oxohept-4-ene-1,7-dioate, 2-OHD=2-oxoheptane-1,7-dioate, 2-AHE=2-aminohept-4-ene-1,7-dioate, 2-AHD=2-aminoheptane-1,7-dioate, and 6-OHE=6-oxohex-4-enoate.

FIG. 7 shows exemplary pathways to hexamethylenediamine from 6-aminocapropate. Enzymes are A) 6-aminocaproate kinase, B) 6-AHOP oxidoreductase, C) 6-aminocaproic semialdehyde aminotransferase and/or 6-aminocaproic semialdehyde oxidoreductase (aminating), D) 6-aminocaproate N-acetyltransferase, E) 6-acetamidohexanoate kinase, F) 6-AAHOP oxidoreductase, G) 6-acetamidohexanal aminotransferase and/or 6-acetamidohexanal oxidoreductase (aminating), H) 6-acetamidohexanamine N-acetyltransferase and/or 6-acetamidohexanamine hydrolase (amide), I) 6-acetamidohexanoate CoA transferase and/or 6-acetamidohexanoate CoA ligase, J) 6-acetamidohexanoyl-CoA oxidoreductase, K) 6-AAHOP acyltransferase, L) 6-AHOP acyltransferase, M) 6-aminocaproate CoA transferase and/or 6-aminocaproate CoA ligase, N) 6-aminocaproyl-CoA oxidoreductase. Abbreviations are: 6-AAHOP=[(6-acetamidohexanoyl)oxy]phosphonate and 6-AHOP=[(6-aminohexanoyl)oxy]phosphonate.

FIG. 8 shows exemplary biosynthetic pathways leading to 1,6-hexanediol. A) is a 6-aminocaproyl-CoA transferase or synthetase catalyzing conversion of 6ACA to 6-aminocaproyl-CoA; B) is a 6-aminocaproyl-CoA reductase catalyzing conversion of 6-aminocaproyl-CoA to 6-aminocaproate semialdehyde; C) is a 6-aminocaproate semialdehyde reductase catalyzing conversion of 6-aminocaproate semialdehyde to 6-aminohexanol; D) is a 6-aminocaproate reductase catalyzing conversion of 6ACA to 6-aminocaproate semialdehyde; E) is an adipyl-CoA reductase adipyl-CoA to adipate semialdehyde; F) is an adipate semialdehyde reductase catalyzing conversion of adipate semialdehyde to 6-hydroxyhexanoate; G) is a 6-hydroxyhexanoyl-CoA transferase or synthetase catalyzing conversion of 6-hydroxyhexanoate to 6-hydroxyhexanoyl-CoA; H) is a 6-hydroxyhexanoyl-CoA reductase catalyzing conversion of 6-hydroxyhexanoyl-CoA to 6-hydroxyhexanal; I) is a 6-hydroxyhexanal reductase catalyzing conversion of 6-hydroxyhexanal to HDO; J) is a 6-aminohexanol aminotransferase or oxidoreductases catalyzing conversion of 6-aminohexanol to 6-hydroxyhexanal; K) is a 6-hydroxyhexanoate reductase catalyzing conversion of 6-hydroxyhexanoate to 6-hydroxyhexanal; L) is an adipate reductase catalyzing conversion of ADA to adipate semialdehyde; and M) is an adipyl-CoA transferase, hydrolase or synthase catalyzing conversion of adipyl-CoA to ADA.

FIG. 9 shows exemplary pathways from adipate or adipyl-CoA to caprolactone. Enzymes are A. adipyl-CoA reductase, B. adipate semialdehyde reductase, C. 6-hydroxyhexanoyl-CoA transferase or synthetase, D. 6-hydroxyhexanoyl-CoA cyclase or spontaneous cyclization, E. adipate reductase, F. adipyl-CoA transferase, synthetase or hydrolase, G. 6-hydroxyhexanoate cyclase, H. 6-hydroxyhexanoate kinase, I. 6-hydroxyhexanoyl phosphate cyclase or spontaneous cyclization, J. phosphotrans-6-hydroxyhexanoylase.

FIG. 10 shows an exemplary hexamethylenediamine (HMD) biosynthetic pathway. Starting from succinyl-CoA and acetyl-CoA the enzymes are designated as follows: (A) thiolase; (B) hydroxyadipyl-CoA dehydrogenase (HBD); (C) crotonase; (D) trans-enoyl-CoA reductase (Ter); (E) 6ACA-aldehyde dehydrogenase (ALD); (F) 6ACA-transaminase (TA); (G) CoA transferase/CoA ligase; (H) HMD-aldehyde dehydrogenase (ALD); (I) carboxylic acid reductase (CAR), and (J) HMD-transaminase (TA2). PPTase corresponds to a phosphopantetheinyl transferase.

FIG. 11 shows the enzymatic activities of the CAR homolog from Mycobacterium avium (SEQ ID NO:153) on four carbon substrates (butyrate, 4-hydroxybutyrate (4-HB, succinate and 4-aminobutyric acid (GABA)) and on six carbon substrates (hexanoate, 6-hydroxycaproic acid, adipate and 6ACA).

FIG. 12 shows the enzymatic activity of the CAR homolog from Mycobacterium avium (SEQ ID NO:153; Parent) compared to variant 1 shown in Table 9.

### DETAILED DESCRIPTION

Unless defined otherwise, all technical and scientific terms used herein have the same meaning as commonly understood by those of ordinary skill in the art to which embodiments of the invention belongs. Any methods, devices and materials similar or equivalent to those described herein can be used in the practice of embodiments of the invention. The following definitions are provided to facilitate understanding of certain terms used frequently herein and are not meant to limit the scope of the present disclosure. All documents (e. g. patent applications or patents) referred to herein are incorporated by reference in their entirety.

Described are aminotransferases, also known as transaminases (E. C. 2. 6. 1) that catalyze the transfer of an amino group, a pair of electrons, and a proton from a primary amine of an amino donor substrate to the carbonyl group of an amino acceptor molecule. The desired reaction of the transaminase is to transfer the amino group of glutamate or alanine to adipate semialdehyde to form 6-aminocaproic acid (6ACA), which is shown below:

Adipate semialdehyde+Glutamate→6ACA+alpha-ketoglutaric acid

However, transaminases also have specificity for succinate semialdehyde or pyruvate as shown below:

Succinate semialdehyde+glutamate→gamma-aminobutyric acid+alpha-ketoglutaric acid

Pyruvate+glutamate→alanine+alpha-ketoglutaric acid

Alanine may substitute for glutamate as the amine donor.

The described transaminase (TA) enzymes identified to be active on an adipate semialdehyde substrate to form 6-aminocaproic acid. This enzyme maybe used in various pathways leading to nylon intermediates.

The desired transaminases were identified by homology search as well as metagenomic discovery for the enzymes that can perform the desired reaction in the pathway to produce 6ACA. To evaluate the transaminase for adipate semialdehyde utilization, in some embodiments, the assay can be conducted in the forward or reverse direction with 6ACA or another candidate substrate as exemplified herein. The assay can be conducted by direct or indirect measurement of the enzymatic product using methods well known in the art. One exemplary method is an indirect method that is exemplified below and in the Examples.

In some embodiments, a transaminase enzyme from Achromobacter xylosoxidans encoded by SEQ ID NO: 1 was identified. To identify other TA enzymes, SEQ ID NO: 1 was used. Homologous enzymes were identified as set out in Table 6. In some embodiments, transaminase enzymes or sequences are identified by BLAST. In some embodiments, the transaminase shares at least about 40%, 45%, 50%, 55%, 60%, 65%, 70%, 75%, 80%, 85%, 90%, 95%, 96%, 97%, 98%, 99% or 100% sequence identity, to at least 25, 50, 75, 100, 150, 200, 250, 300 or more contiguous amino acids of the amino acid sequences of the transaminases of Table 6.

In some embodiments, the transaminases identified in Table 6 share at least about 40%, 45%, 50%, 55%, 60%, 65%, 70%, 75%, 80%, 85%, 90%, 95%, 96%, 97%, 98%, 99% or 100% sequence identity, to at least 25, 50, 75, 100, 150, 200, 250, 300 or more contiguous amino acids of the amino acid sequences of the transaminases of SEQ ID NO:1, 13 or 31.

In some embodiments, the transaminase enzyme has at least about 50% amino acid sequence identity to at least 25, 50, 75, 100, 150, 200, 250, 300, or more contiguous amino acids of any of SEQ ID NOs: 1, 3, 4, 5, 9, 12, 13, 26, 27, 30, 31, 38, 50, 52, 64, 74, 78, 79, 81, 91, 106, 108, and 116. In some embodiments the amino acid sequence of the transaminase enzyme that reacts with adipate semialdehyde to form 6ACA is selected from the amino acid sequences of SEQ ID NOS: 1, 3, 4, 5, 9, 12, 13, 26, 27, 30, 31, 38, 50, 52, 64, 74, 78, 79, 81, 91, 106, 108, and 116.

In some embodiments, the TA enzymes catalytic efficiency, and/or turnover number for adipate semialdehyde as the substrate is similar to when succinate semialdehyde is the substrate. In some embodiments the enzymes with catalytic efficiency, and/or turnover number for adipate semialdehyde as the substrate that is similar to when succinate semialdehyde share at least about 40%, 45%, 50%, 55%, 60%, 65%, 70%, 75%, 80%, 85%, 90%, 95%, 96%, 97%, 98%, 99% or 100% sequence identity, to at least 25, 50, 75, 100, 150, 200, 250, 300 or more contiguous amino acids of the amino acid sequences of the transaminase of SEQ ID NO: 1, 3, 4, 5, 9, 12, 13, 26, 27, 30, 31, 38, 50, 52, 64, 74, 78, 79, 81, 91, 106, 108, and 116.

As used with respect to any of the enzymes described herein, the term turnover number (also termed as k_{cat}) is defined as the maximum number of chemical conversions of substrate molecules per second that a single catalytic site will execute for a given enzyme concentration [E_{T}]. It can be calculated from the maximum reaction rate Vmax and catalyst site concentration [E_{T}] as follows:
Kcat=Vmax/[E_{T}]. The unit is s⁻¹.

As used with respect to any of the enzymes described herein, the term “catalytic efficiency” is a measure of how efficiently an enzyme converts substrates into products. A comparison of catalytic efficiencies can also be used as a measure of the preference of an enzyme for different substrates (i. e., substrate specificity). The higher the catalytic efficiency, the more the enzyme “prefers” that substrate. It can be calculated from the formula: k_{cat}/K_{M}, where k_{cat} is the turnover number and K_{M} is the Michaelis constant, K_{M} is the substrate concentration at which the reaction rate is half of Vmax. The unit of catalytic efficiency can be expressed as s⁻¹M⁻¹.

The transaminase enzymes identified are derived from very genetically diverse organisms. Shown below are the pairwise sequence alignments of some exemplary transaminases are shown Table 1.

TABLE 1

| | SEQ ID NO: 1 | SEQ ID NO: 13 | SEQ ID NO: 31 |
|---|---|---|---|
| SEQ ID NO: 1 | | 44% | 87% |
| SEQ ID NO: 13 | 44% | | 43% |
| SEQ ID NO: 31 | 87% | 43% | |

The transaminase enzymes have conserved domains. Based on the multiple sequence alignments and hidden Markov models (HMMs), the transaminase enzymes are grouped into Pfam PF00202, of the Pfam database from the European Bioinformatics Institute (pfam.xfam.org).

In some embodiments, amino acid positions were identified for mutation in SEQ ID NO:1 by examination of the crystal structure of the protein, and the gene encoding SEQ ID NO:1 was subjected to saturation mutagenesis at selected amino acid positions. Catalytically-relevant residues were identified that can be subject to change to provide a variant amino acid with activity better than the wild-type (unmodified) SEQ ID NO: 1.

In some embodiments, transaminase enzymes are engineered to have greater specificity for the adipate semialdehyde substrate than its corresponding wild-type.

As used herein “engineered” or “variant” when used in reference to any polypeptide or nucleic acid described here refers to a sequence having at least one variation or alteration at an amino acid position or nucleic acid position as compared to a parent sequence. The parent sequence can be, for example, an unmodified, wild-type sequence, a homolog thereof or a modified variant of, for example, a wild-type sequence or homolog thereof.

In some embodiments, the engineered transaminase has one or more alterations of an amino acid of SEQ ID NO; 1, SEQ ID NO: 13, or SEQ ID NO: 31. In some embodiments the engineered transaminase has alterations in amino acid sequences that have at least one, at least two, at least three, at least four, at least five, at least six, at least seven, or at least eight alterations of an amino acid with respect to SEQ ID NO:1 SEQ ID NO: 13 or SEQ ID NO: 31.

In some embodiments, the engineered TA has one or more amino acid alterations selected from one or more positions corresponding to residues V114, S136, T148, P153, I203, I204, P206, V207, V111, T216, A237, T264, M265 and L386, G19, C22, D70, R94, D99, T109, E112, A113, F137, G144, I149, K150, Y154, S178, L186, Q208, L234, T242, A315, K318, R338, G336, L386, V390, A406, S416, A421, or one or more combinations of the amino acid alterations and amino acid residue positions of SEQ ID NO:1.

In some embodiments, the one or more amino acid alteration of the engineered protein is a substitution of a conservative or non-conservative amino acid at one or more positions corresponding to residue V114, S136, T148, P153, I203, I204, P206, V207, V111, T216, A237, T264, M265 and L386, G19, C22, D70, R94, D99, T109, E112, A113, F137, G144, I149, K150, Y154, S178, L186, Q208, L234, T242, A315, K318, R338, G336, L386, V390, A406, S416, and A421 or one or more combinations of the amino acid alterations and amino acid residue positions of SEQ ID NO:1.

In some embodiments, the engineered TA has one or more amino acid alterations of the engineered protein is an alteration at a positions corresponding to the residues shown in Table 7.

In some embodiments, the engineered TA enzyme has at least a catalytic efficiency for adipate semialdehyde substrate that is at least 1.5×, at least 2×, at least 5×, at least 10×, at least 25×, or 1.5-25× as compared to the corresponding wild-type enzyme having SEQ ID NOs: 1, 13, or 31.

In some embodiments, the enzymatic conversion of adipate semialdehyde by the engineered transaminase enzyme under known standard conditions is at least 10, at least 20, at least 30, at least 40, at least 50, at least 60, at least 70, at least 80, or at least 90 percent more than the enzymatic activity of the enzyme for the corresponding wild-type enzyme having SEQ ID SEQ ID NOs: 1, 13, or 31.

In some embodiments, to provide a TA variant, Achromobacter xylosoxidans TA is represented by SEQ ID NO: 1 of the disclosure is selected as a template or parent sequence. Variants, as described herein, can be created by introducing into the template one or more amino acid alterations (e.g. substitutions). The variants can be screened to identify those that have increased activity and/or specificity for their substrates. For example, a TA variant is screened to identify those alterations leading to increased activity and/or specificity for adipate semialdehyde or analogs thereof. Other variants described herein would similarly be screened to identify increased activity and/or specificity for the parent enzyme's substrate or substrates.

For the purpose of amino acid position numbering, in some embodiments, SEQ ID NO: 1 is used as the reference sequence. Therefore, for example, mention of amino acid position 79 in reference to SEQ ID NO:1, but in the context of a different TA sequence (a target sequence or other template sequence) the corresponding amino acid position for variant creation may have the same or different position number, (e.g. 78, 79 or 80). In some cases, the original amino acid and its position on the SEQ ID NO: 1 reference template will precisely correlate with the original amino acid and position on the target TA sequence. In other cases, the original amino acid and its position on the SEQ ID NO: 1 reference template will correlate with the original amino acid, but its position on the target will not be in the corresponding template position. However, the corresponding amino acid on the target can be a predetermined distance from the position on the template, such as within 10, 9, 8, 7, 6, 5, 4, 3, 2, or 1 amino acid positions from the reference template position. In other cases, the original amino acid on the SEQ ID NO: 1 reference template will not precisely correlate with the original amino acid on the target. However, one can understand what the corresponding amino acid on the target sequence is based on the general location of the amino acid on the template and the sequence of amino acids in the vicinity of the target amino acid. It is understood that sequence alignments can be generated with TA sequences not specifically disclosed herein, and such alignments can be used to understand and generate new TA variants given the teachings and guidance of the current disclosure. In some embodiments, the sequence alignments can allow one to understand common or similar amino acids in the vicinity of the target amino acid, and those amino acids can be viewed as “sequence motif” having a certain amount of identity or similarity to between the template and target sequences. Those sequence motifs can be used to describe portions of TA sequences where variant amino acids are located, and the type of variation(s) that can be present in the motif.

Also, described are carboxylic acid reductases (CAR) that catalyze the ATP and NADPH-dependent reduction of carboxylic acids to their corresponding aldehydes (Venkitasubramanian et al., J. Biol. Chem. 282:478-485 (2007)). The CARs described herein can be used to convert the intermediate 6ACA to 6-aminocaproate semialdehyde and have E.C. number E.C. 1.2.1.30. 6ACA and 6-aminocaproate semialdehyde are intermediates in, and the conversion of 6ACA to 6-aminocaproate semialdehyde is and enzymatic step in, hexamethylenediamine (HMD) and hexanediol (HDO) pathways described herein. Accordingly, the CARs can be utilized in various pathways leading to nylon intermediates including, for example, the HMD and HDO pathways described herein.

The desired CARs were identified by homology search as well as metagenomic discovery for the enzymes that can perform the desired reaction in the pathway to produce 6-aminocaproate semialdehyde. To evaluate the CAR for 6ACA utilization, in some embodiments, the assay can be conducted in the forward direction with 6ACA or another candidate substrate as exemplified herein. Similarly, the assay also can be conducted in the reverse direction with 6-aminocaproate semialdehyde or another candidate substrate. The assay can be conducted by direct or indirect measurement of the enzymatic product using methods well known in the art. One exemplary method is an indirect method that is exemplified below and in the Examples.

In some embodiments, a CAR enzyme from Mycolicibacterium smegmatis MC2 155 encoded by SEQ ID NO:150 was identified. In some embodiments, a CAR enzyme from Mycobacterium avium encoded by SEQ ID NO:153 was identified. To identify other CAR enzymes, SEQ ID NO:150 and SEQ ID NO:153 were used. Homologous enzymes were identified as set out in Table 8. In some embodiments, CAR enzymes or sequences are identified by BLAST. In some embodiments, the CAR shares at least about 40%, 45%, 50%, 55%, 60%, 65%, 70%, 75%, 80%, 8%, 90%, 95%, 96%, 97%, 98%, 99% or 100% sequence identity, to at least 25, 50, 75, 100, 150, 200, 250, 300 or more contiguous amino acids of the amino acid sequences of the CARs of Table 8.

In some embodiments, the CARs identified in Table 8 share at least about 40%, 45%, 50%, 55%, 60%, 65%, 70%, 75%, 80%, 85%, 90%, 95%, 96%, 97%, 98%, 99% or 100% sequence identity, to at least 25, 50, 75, 100, 150, 200, 250, 300 or more contiguous amino acids of the amino acid sequences of the CARs of SEQ ID NOS:152, 153 or 254.

In some embodiments, the CAR enzyme has at least about 50% amino acid sequence identity to at least 25, 50, 75, 100, 150, 200, 250, 300, or more contiguous amino acids of any of SEQ ID NOS:150-165, 168-171, 173-178, 180, 183-185, 187-188, 190-193, 195, 198-200, 202-216, 218-219, 221-230, 232-238, 241-244, 246-249, 251-252 and 255-264. In some embodiments the amino acid sequence of the CAR enzyme that reacts with 6ACA to form 6-aminocproate semialdehyde is selected from the amino acid sequences of SEQ ID NOS:150-165, 168-171, 173-178, 180, 183-185, 187-188, 190-193, 195, 198-200, 202-216, 218-219, 221-230, 232-238, 241-244, 246-249, 251-252 and 255-264.

In some embodiments, the CAR enzymes catalytic efficiency, and/or turnover number for 6ACA as the substrate is similar to when succinate is the substrate. In some embodiments, the CAR enzymes catalytic efficiency and/or turnover number for 6ACA as the substrate is reduced compared to when hexanoate is the substrate. In other embodiments, the CAR enzymes with catalytic efficiency, and/or turnover number for 6ACA as the substrate that is similar to when succinate is the substrate share at least about 40%, 45%, 50%, 55%, 60%, 65%, 70%, 75%, 80%, 85%, 90%, 95%, 96%, 97%, 98%, 99% or 100% sequence identity, to at least 25, 50, 75, 100, 150, 200, 250, 300 or more contiguous amino acids of the amino acid sequences of the CARs of SEQ ID NO:150-165, 168-171, 173-178, 180, 183-185, 187-188, 190-193, 195, 198-200, 202-216, 218-219, 221-230, 232-238, 241-244, 246-249, 251-252 and 255-264. In still other embodiments, the CAR enzymes with catalytic efficiency and/or turnover number for 6ACA as the substrate is reduced compared to when hexanoate is the substrate share at lease about 40%, 45%, 50%, 55%, 60%, 65%, 70%, 75%, 80%, 85%, 90%, 95%, 96%, 97%, 98%, 99% or 100% sequence identity, to at least 25, 50, 75, 100, 150, 200, 250, 300 or more contiguous amino acids of the amino acid sequences of the CARs of SEQ ID NOS:150-165, 168-171, 173-178, 180, 183-185, 187-188, 190-193, 195, 198-200, 202-216, 218-219, 221-230, 232-238, 241-244, 246-249, 251-252 and 255-264.

The CAR enzymes identified are derived from very genetically diverse organisms. Shown below are the pairwise sequence alignments of some exemplary CARs are shown Table 2.

TABLE 2
Percent identity in pairwise sequence
alignments of exemplary CARs

| | SEQ ID | SEQ ID | SEQ ID | SEQ ID |
|---|---|---|---|---|
| | NO: 152 | NO: 153 | NO: 159 | NO: 254 |
| SEQ ID NO: 152 | | 99% | 34% | 74% |
| SEQ ID NO: 153 | 99% | | 34% | 75% |
| SEQ ID NO: 159 | 34% | 34% | | 36% |
| SEQ ID NO: 254 | 74% | 75% | 36% | |

The CAR enzymes have conserved domains. Based on the multiple sequence alignments and hidden Markov models (IMMs), the CAR enzymes can comprise the following domains of the Pfam database from the European Bioinformatics Institute (pfam.xfam.org): an AMP-binding domain (Pfam PF00501), a NAD-binding domain (Pfam PF07993), and a phosphopantetheine (PP)-binding domain (Pfam PF00550).

In some embodiments, amino acid positions were identified for mutation in SEQ ID NO:152 by examination of the crystal structure of the protein, and the gene encoding SEQ ID NO:152 or a homolog thereof was subjected to saturation mutagenesis at selected amino acid positions. Catalytically-relevant residues were identified that can be subject to change to provide a variant amino acid with activity better than the wild-type (unmodified) SEQ ID NO:152 or a homolog thereof. In some embodiments, amino acid positions were identified for mutation in SEQ ID NO:153, and the gene encoding SEQ ID NO:153 or a homolog thereof was used as a template for protein engineering (e.g., subjected to mutagenesis at selected amino acid positions). In some embodiments, amino acid positions were identified for mutation in SEQ ID NO:254, and the gene encoding SEQ ID NO:254 or a homolog thereof was used as a template for protein engineering (e.g., subjected to mutagenesis at selected amino acid positions).

In some embodiments, CAR enzymes are engineered to have greater specificity for the 6ACA substrate than its corresponding wild-type.

In some embodiments, the engineered CAR has one or more alterations of an amino acid of SEQ ID NO:152, SEQ ID NO:153 or SEQ ID NO:254. In some embodiments the engineered CAR has alterations in amino acid sequences that have at least one, at least two, at least three, at least four, at least five, at least six, at least seven, or at least eight alterations of an amino acid with respect to SEQ ID NO:152, SEQ ID NO:153 or SEQ ID NO:254. In some embodiments, the engineered CAR has one or more amino acid alterations selected from one or more positions corresponding to residues P141, L245, I247, W270, S274, K275, N276, F278, G279, N279insert, A282, A283, S299, I300, N335, S336, M389, G391, G414, G421, M422, F425, G636, D809, I810, L811, A812 and F929, or one or more combinations of the amino acid alterations and amino acid residue positions of SEQ ID NO:152, 153 or 254.

In some embodiments, the engineered CAR comprises one or more amino acid alterations at one or more residue positions selected from the group consisting of: N335E; N335D; S274D; S274E; K275D; K275E; S299D; S299E; M389D; M389E; G414D; G414E; G421D; G421E; M422D; M422E; F425D; F425E; N335D and A282P; N335D and A282V; N335D and A283C; N335D, A283C and F929L; N335D, A283C and G636D; N335D and A283G; N335D and F278A; N335D and F278C; N335D and F278S; N335D and F278V; N335D and G279V; N335D and I247M; N335D and I247Q; N335D and I247T; N335D and I247V; N335D and I300C; N335D and I300G; N335D and I300M; N335D and I300M; N335D and I300Y; N335D and K275A; N335D and K275D; N335D and K275D; N335D and K275E; N335D and K275M; N335D and K275N; N335D and K275S; N335D and K275T; N335D and K275V; N335D and K275W; N335D and L245C; N335D and L245G; N335D and L245S; N335D and L245T; N335D and L245V; N335D and M389I; N335D and M389W; N335D and M422D; N335D and N276S; N335D and N279 INSERT; N335D and P141G; N335D and S299D; N335D and S299E; N335D and S391G; N335D and W270M; K275D, N276S, F278S, A283C and N335D; L245V, K275D, N276S, F278S, A283C and N335D; I247M, K275D, N276S, F278A, A283C and N335D; L245V, K275T, N276S, F278S, A283C and N335D; I247M, K275T, N276S, F278S, A283C and N335D; K275D, N276S, F278S, A283C, I300G and N335D; L245T, K275D, N276S, F278A, A283C and N335D; K275N, F278S, A283C, S299D and N335D; I247M, K275N, N276S, F278S, A283C and N335D; K275N, F278S, A283C, I300G and N335D; K275N, N276S, F278S, A283C, I300G and N335D; I247M, K275T, F278S, A283C and N335D; L245V, K275D, N276S, F278A, A283C and N335D; K275N, F278S, A283C, I300G and N335D; K275N, F278A, A283C, S299D and N335D; K275N, N276S, F278A, A283C and N335D; L245V, K275N, N276S, F278S, A283C, I300G and N335D; L245T, K275N, N276S, F278S, A283C, I300G and N335D; K275N, F278A, S299D and N335D; I247M, K275N, F278A, A283C, I300Y and N335D; I247M, K275N, F278A, A283C and N335D; K275D, N276S, F278A and N335D; K275T, F278A, A283C, S299D and N335D; F278A, A282P and N335D; F278A, A283C and N335D; K275N, S299D and N335D; L245T, S299D and N335D; K275D, S299D and N335D; K275N, F278S and N335D; S299D, M389W and N335D; G279V, S299D and N335D; F278A, S283C and N335D; K275D, N276S and N335D; G279V, S299E and N335D; I247M, S299D and N335D; P141G, A282P and N335D; L245T, A282P and N335D; F278S, A283C and N335D; K275D, S284I, S299D and N335D; I247M, I282P and N335D; I247V, K275D, N276S, F278S, A283C, I300G and N335D; I247V, K275D, N276S, F278S, A283C, I300G and N335D; K275D, S274A, N276S, F278S, A283C, I300G and N335D; K275D, S274P, N276S, F278S, A283C, I300G and N335D; K275D, N276S, F278S, A282F, A283C, I300G and N335D; K275D, N276S, F278S, A282P, A283C, I300G and N335D; K275D, N276S, F278S, A283C, S299I, I300G and N335D; K275D, N276S, F278S, A283C, I300G, N335D and M389C; K275D, N276S, F278S, A283C, I300G, N335D and M389Y; and K275D, N276S, F278S, A283C, I300G, N335D and M389S.

In some embodiments, the one or more amino acid alteration of the engineered protein is a substitution of a conservative or non-conservative amino acid at one or more positions corresponding to residue P141, L245, I247, W270, S274, K275, N276, F278, G279, N279insert, A282, A283, S299, I300, N335, S336, M389, G391, G414, G421, M422, F425, G636, D809, I810, L811, A812 and F929 or one or more combinations of the amino acid alterations and amino acid residue positions of SEQ ID NO:152, 153 or 254.

In some embodiments, the one or more amino acid alterations of the engineered protein is an alteration at a positions corresponding to the residues shown in Table 9.

In some embodiments, the engineered CAR enzyme has at least a catalytic efficiency for 6ACA substrate that is at least 1.5×, at least 2×, at least 5×, at least 10×, at least 25×, or 1.5-25× as compared to the corresponding wild-type or parent enzyme having SEQ ID NOS:152, 153, or 254.

In some embodiments, the enzymatic conversion of 6ACA by the engineered CAR enzyme under known standard conditions is at least 10, at least 20, at least 30, at least 40, at least 50, at least 60, at least 70, at least 80, or at least 90 percent more than the enzymatic activity of the enzyme for the corresponding wild type or parent enzyme having SEQ ID SEQ ID NOS:152, 153 or 254.

In some embodiments, to provide a CAR variant, Mycobacterium avium or homolog thereof CAR is represented by SEQ ID NOS:152 and 153 of the disclosure is selected as a template or parent sequence. In some embodiments, to provide a CAR variant, Mycobacterium sp. JS623 or homolog thereof CAR is represented by SEQ ID NO:254 of the disclosure is selected as a template or parent sequence. CAR variants described herein can be screened to identify those alterations leading to increased activity and/or specificity for 6ACA or other candidate substrate as exemplified herein.

For the purpose of amino acid position numbering for CARs described herein, in some embodiments, SEQ ID NO:152 is used as the reference sequence. Therefore, for example, mention of amino acid position 89 in reference to SEQ ID NO:152, but in the context of a different CAR sequence (a target sequence or other template sequence) the corresponding amino acid position for variant creation can have the same or different position number, (e.g. 88, 89 or 90). In some cases, the original amino acid and its position on the SEQ ID NO:152 reference template will precisely correlate with the original amino acid and position on the target CAR sequence. In other cases, the original amino acid and its position on the SEQ ID NO:152 reference template will correlate with the original amino acid, but its position on the target will not be in the corresponding template position. However, the corresponding amino acid on the target can be a predetermined distance from the position on the template, such as within 10, 9, 8, 7, 6, 5, 4, 3, 2, or 1 amino acid positions from the reference template position. In other cases, the original amino acid on the SEQ ID NO:152 reference template will not precisely correlate with the original amino acid on the target. However, one can understand what the corresponding amino acid on the target sequence is based on the general location of the amino acid on the reference template and the sequence of amino acids in the vicinity of the target amino acid. It is understood that sequence alignments can be generated with CAR sequences not specifically disclosed herein, and such alignments can be used to understand and generate new CAR variants given the teachings and guidance of the current disclosure. In some embodiments, the sequence alignments can allow one to understand common or similar amino acids in the vicinity of the target amino acid, and those amino acids can be viewed as “sequence motif” having a certain amount of identity or similarity to between the template and target sequences. Those sequence motifs can be used to describe portions of CAR sequences where variant amino acids are located, and the type of variation(s) that can be present in the motif.

In some embodiments, the engineered CAR has one or more amino acid alterations of the engineered protein, wherein the one or more alterations is an alteration at a positions corresponding to the residues shown in Table 10. In some embodiments the engineered CAR has alterations in amino acid sequences that have at least one, at least two, at least three, at least four, at least five, at least six, at least seven, or at least eight alterations of an amino acid with respect to SEQ ID NO:153 as shown in Table 10. In some embodiments, the engineered CAR has one or more amino acid alterations shown in Table 10 in addition to the amino acid alteration N335D. In some embodiments, the engineered CAR has one or more amino acid alterations selected from one or more positions corresponding to residues for L245, I247, S274, K275, N276, F278, A282, A283, S299, I300, and M389 of SEQ ID NO:153. In some embodiments, the engineered CAR can comprise a L, T or V at position 245 of SEQ ID NO:153. In some embodiments, the engineered CAR can comprise a I, M or T at position 247 of SEQ ID NO:153. In some embodiments, the engineered CAR can comprise a S, C, A, or P at position 274 of SEQ ID NO:153. In some embodiments, the engineered CAR can comprise a K, D, N, or T at position 275 of SEQ ID NO:153. In some embodiments, the engineered CAR can comprise a N or S at position 276 of SEQ ID NO:153. In some embodiments, the engineered CAR can comprise a F, S or A at position 278 of SEQ ID NO:153. In some embodiments, the engineered CAR can comprise a A, F, or P at position 282 of SEQ ID NO:153. In some embodiments, the engineered CAR can comprise a A or C at position 283 of SEQ ID NO:153. In some embodiments, the engineered CAR can comprise a S or D at position 299 of SEQ ID NO:153. In some embodiments, the engineered CAR can comprise a I, G, or Y at position 300 of SEQ ID NO:153. In some embodiments, the engineered CAR can comprise a M, C, Y, or S at position 389 of SEQ ID NO:153. Mutations can be made singly and in combination with mutations at other amino acid positions shown in Table 10.

Additionally, described are transaminases (E.C.2.6.1) different from the TA transaminase described above. These transaminase are referred to herein as TA2 transaminase, transaminase TA2 or TA2 and can be used to convert 6-aminocaproate semialdehyde to HMD. 6-aminocaproate semialdehyde is an intermediate in, and the conversion of 6-aminocaproate to HMD is an enzymatic step in HMD pathways described herein. Accordingly, TA2 can be utilized in various pathways leading to nylon intermediates including, for example, the HMID pathways described herein.

The desired TA2 transaminases were identified by homology search as well as metagenomic discovery for the enzymes that can perform the desired reaction in the pathway to produce hexamethylenediamine (HMD). To evaluate the TA2 for 6-aminocaproate semialdehyde utilization, in some embodiments, the assay can be conducted in the forward direction with 6-aminocaproate semialdehyde or another candidate substrate as exemplified herein. Similarly, the assay also can be conducted in the reverse direction with HMD or another candidate substrate. The assay also can be conducted using 6ACA as with the TA transaminases. Those TA2 transaminases active with 6ACA can then be screened for activity in conversion of 6-aminocaproate semialdehyde to HND. The assay can be conducted by direct or indirect measurement of the enzymatic product using methods well known in the art. One exemplary method is an indirect method that is exemplified below and in the.

In some embodiments, a TA2 enzyme from Escherichia coli encoded by SEQ ID NO:265 was identified. To identify other TA2 enzymes, SEQ ID NO:265 was used. Homologous enzymes were identified as set out in Table 11. In some embodiments, TA2 enzymes or sequences are identified by BLAST. In some embodiments, the TA2 shares at least about 40%, 45%, 50%, 55%, 60%, 65%, 70%, 75%, 80%, 85%, 90%, 95%, 96%, 97%, 98%, 99% or 100% sequence identity, to at least 25, 50, 75, 100, 150, 200, 250, 300 or more contiguous amino acids of the amino acid sequences of the TA2 of Table 11.

In some embodiments, the TA2s identified in Table 11 share at least about 40%, 45%, 50%, 55%, 60%, 65%, 70%, 75%, 80%, 85%, 90%, 95%, 96%, 97%, 98%, 99% or 100% sequence identity, to at least 25, 50, 75, 100, 150, 200, 250, 300 or more contiguous amino acids of the amino acid sequences of the TA2 of SEQ ID NO:265.

In some embodiments, the TA2 enzyme have at least about 50% amino acid sequence identity to at least 25, 50, 75, 100, 150, 200, 250, 300, or more contiguous amino acids of any of SEQ ID NOS:265 and 267-296. In some embodiments the amino acid sequence of the TA2 enzyme that reacts with 6-aminocaproate semialdehyde to form HMD are selected from the amino acid sequences of SEQ ID NOS:265 and 267-296.

In some embodiments, the TA2 enzymes catalytic efficiency, and/or turnover number for 6-aminocaproate semialdehyde as the substrate is similar to when 6ACA is the substrate. In some embodiments the enzymes with catalytic efficiency, and/or turnover number for 6-aminocaproate semialdehyde as the substrate that is similar to when 6ACA is the substrate share at least about 40%, 45%, 50%, 55%, 60%, 65%, 70%, 75%, 80%, 85%, 90%, 95%, 96%, 97%, 98%, 99% or 100% sequence identity, to at least 25, 50, 75, 100, 150, 200, 250, 300 or more contiguous amino acids of the amino acid sequences of any of the TA2 of SEQ ID NO:265 and 267-296.

The TA2 enzymes identified are derived from very genetically diverse organisms. Shown below are the pairwise sequence alignments of some exemplary TA2s are shown Table 3.

TABLE 3
Percent identity in pairwise sequence
alignments of exemplary TA2s

| | SEQ ID | SEQ ID | SEQ ID | SEQ ID |
|---|---|---|---|---|
| | NO: 265 | NO: 270 | NO: 289 | NO: 296 |
| SEQ ID NO: 265 | | 45% | 61% | 95% |
| SEQ ID NO: 270 | 45% | | 47% | 47% |
| SEQ ID NO: 289 | 61% | 47% | | 61% |
| SEQ ID NO: 296 | 95% | 47% | 61% | |

The TA2 enzymes have conserved domains. Based on the multiple sequence alignments and hidden Markov models (HMMs), the TA2 enzymes are grouped into Pfam PF00202, of the Pfam database from the European Bioinformatics Institute (pfam.xfam.org). TA2 enzymes have conserved lysine residues in the active site for pyridoxal phosphate (PLP) binding. The lysine residue and the aldehyde group of PLP can form a Schiff-base structure, resulting in an active conformation.

In one aspect, provided are a non-naturally occurring microbial organism comprising an exogenous nucleic acid encoding: (a) an engineered carboxylic acid reductase (CAR) enzyme comprising at least one alteration of an amino acid of SEQ ID NOS:152, 153 or 254; (b) a CAR comprising an amino acid sequence having at least 50% sequence identity to at least 25, 50, 75, 100, 150, 200, 250, 300, or more contiguous amino acids of any of SEQ ID NOS:150-165, 168-171, 173-178, 180, 183-185, 187-188, 190-193, 195, 198-200, 202-216, 218-219, 221-230, 232-238, 241-244, 246-249, 251-252 and 255-264; or (c) a hexamethylenediamine (HMD) transaminase (TA2) enzyme having at least 50% sequence identity to at least 25, 50, 75, 100, 150, 200, 250, 300, or more contiguous amino acids of any of SEQ ID NOS:265 and 267-296; and further comprising (d) at least one exogenous nucleic acid encoding an aldehyde dehydrogenase (ALD) enzyme that reacts with adipyl-CoA to form adipate-semialdehyde, wherein the aldehyde dehydrogenase has greater catalytic efficiency for adipyl-CoA substrate as compared to succinyl-CoA, acetyl-CoA, or both succinyl-CoA and acetyl-CoA substrates, and/or the aldehyde dehydrogenase has higher turnover number for adipyl-CoA substrate as compared to succinyl-CoA, acetyl-CoA, or both succinyl-CoA and acetyl-CoA substrates.

In some embodiments, the one exogenous nucleic acid encoding an ALD enzyme is integrated into the genome of the non-naturally occurring microbial organism. In some embodiments, the exogenous nucleic acid encoding an ALD enzyme is not integrated into the genome of the microbial organism, e.g., a plasmid. In some embodiments, the exogenous nucleic acid encoding an ALD enzyme is heterologous to the microbial organism.

In some embodiments, the expression of at least one exogenous nucleic acid encoding an ALD enzyme in the non-naturally occurring microbial organism comprising genes encoding a 3-oxoadipyl-CoA thiolase (Thl), a 3-oxoadipyl-CoA dehydrogenase (Hbd), and a 3-oxoadipyl-CoA dehydratase (“crotonase” or Crt), a 5-carboxy-2-pentenoyl-CoA reductase (Ter), and a transaminase (TA), hexamethylenediamine (HMD) transaminase (TA2) and carboxylic acid reductase (CAR) increases the production of HMD as compared to a control microorganism comprising genes encoding a 3-oxoadipyl-CoA thiolase (Thl), a 3-oxoadipyl-CoA dehydrogenase (Hbd), and a 3-oxoadipyl-CoA dehydratase (“crotonase” or Crt), a 5-carboxy-2-pentenoyl-CoA reductase (Ter), and a transaminase (TA), hexamethylenediamine (HMD) transaminase (TA2) and carboxylic acid reductase (CAR) without exogenous nucleic acid encoding an ALD enzyme.

To identify ALD enzymes with greater catalytic efficiency, greater turnover number or both for adipyl CoA substrate than for succinyl-CoA, acetyl CoA, or both substrates, an exemplary sequence of Clostridium kluyveri DSM555, encoded by the gene adh (SEQ ID NO: 141) was used to identify other aldehyde dehydrogenase enzymes. Homologous enzymes were identified as set forth in Table 4.

In some embodiments, aldehyde dehydrogenase enzymes or sequences are identified by BLAST. In some embodiments, the aldehyde dehydrogenase share at least about 40%, 45%, 50%, 55%, 60%, 65%, 70%, 75%, 80%, 85%, 90%, 95%, 96%, 97%, 98%, 99% or 100% sequence identity, to at least 50, 75, 100, 150, 200, 250, 300, or more contiguous amino acids of the amino acid sequences of the ALDs of Table 4.

These ALD enzymes are derived from very genetically diverse organisms. Often a simple amino acid sequence identity between the sequences is not indicative of their common function. For example, the pairwise sequence alignment results of some exemplary aldehyde dehydrogenases disclosed in Table 4 are shown below in Table 12.

TABLE 12
Percent identity in pairwise sequence
alignments of exemplary ALDs

| | SEQ ID | SEQ ID | SEQ ID | SEQ ID |
|---|---|---|---|---|
| | NO: 298 | NO: 306 | NO: 323 | NO: 347 |
| SEQ ID NO: 298 | | 50% | 56% | 60% |
| Source organism: | | | | |
| Peptostreptococcaceae | | | | |
| bacterium oral | | | | |
| SEQ ID NO: 306 | 50% | | 53% | 57% |
| Source organism: | | | | |
| Acidaminococcus | | | | |
| massiliensis | | | | |
| SEQ ID NO: 323 | 56% | 53% | | 60% |
| Source organism: | | | | |
| Collinsella sp. GD7 | | | | |
| SEQ ID NO: 347 | 60% | 67% | 60% | |
| Source organism: | | | | |
| Romboutsia | | | | |
| lituseburensis DSM | | | | |

These ALD enzymes have multiple conserved domains, for example, N-terminal domain, C-terminal domain, and a cysteine residue at its active site. The ALD comprise a cofactor binding domain with a Rossmann-fold type nucleotide binding architecture. The Rossmann fold, also called βαβ fold, is a super-secondary structure that is characterized by an alternating motif of beta-strand-alpha helix-beta strand secondary structures. The β-strands participate in the formation of a β-sheet. The Pap fold structure is commonly observed in enzymes that have dinucleotide coenzymes, such as FAD, NAD and NADP. The Pap fold structure was associated with a specific Gly-rich sequence of (GxGxxG) at the region of the tight loop between the first β-strand the α-helix. In addition, the cofactor binding domain is also the same domain that binds the substrate CoA. It is typical feature of ALDs, where the substrate CoA binds first, forms the intermediate, then the cofactor binds and completes the chemistry and performs the hydride transfer.

Based on the multiple sequence alignments and hidden Markov models (HMMs), the ALD enzymes are grouped into Pfam PF00171, Clan CL0099 of the Pfam database from the European Bioinformatics Institute (pfam.xfam.org). These enzymes are classified as EC 1.2.1 according to the Enzyme Commission nomenclature.

For any of the enzymes described here, in some cases it can be useful to use the Basic Local Alignment Search Tool (BLAST) algorithm to understand the sequence identity between an amino acid motif in a template sequence and a target sequence. Therefore, in preferred modes of practice, BLAST is used to identify or understand the identity of a shorter stretch of amino acids (e. g. a sequence motif) between a template and a target protein. BLAST finds similar sequences using a heuristic method that approximates the Smith-Waterman algorithm by locating short matches between the two sequences. The (BLAST) algorithm can identify library sequences that resemble the query sequence above a certain threshold. Exemplary parameters for determining relatedness of two or more sequences using the BLAST algorithm, for example, can be as set forth below. Briefly, amino acid sequence alignments can be performed using BLASTP version 2. 0. 8 (Jan. 5, 1999) and the following parameters:Matrix: 0 BLOSUM62; gap open: 11; gap extension: 1; x_dropoff: 50; expect: 10. 0; wordsize: 3; filter: on. Nucleic acid sequence alignments can be performed using BLASTN version 2. 0. 6 (Sep. 16, 1998) and the following parameters: Match: 1; mismatch: −2; gap open: 5; gap extension: 2; x_dropoff: 50; expect: 10. 0; wordsize: 11; filter: off. Those skilled in the art will know what modifications can be made to the above parameters to either increase or decrease the stringency of the comparison, for example, and determine the relatedness of two or more sequences.

Site-directed mutagenesis or sequence alteration (e. g., site-specific mutagenesis or oligonucleotide-directed) can be used to make specific changes to a target TA DNA sequence to provide a variant DNA sequence encoding TA with the desired amino acid substitution. As a general matter, an oligonucleotide having a sequence that provides a codon encoding the variant amino acid is used. In some embodiments, artificial gene synthesis of the entire coding region of the variant TA DNA sequence is performed as preferred TA targeted for substitution are generally less than 150 amino acids long.

Exemplary techniques using mutagenic oligonucleotides for generation of a variant TA or CAR sequence include the Kunkel method which may utilize a TA or CAR gene sequence placed into a phagemid. The phagemid in E. coli TA ssDNA which is the template for mutagenesis using an oligonucleotide which is a primer extended on the template.

Depending on the restriction enzyme sites flanking a location of interest in the TA or CAR DNA, cassette mutagenesis may be used to create a variant sequence of interest. For cassette mutagenesis, a DNA fragment is synthesized inserted into a plasmid, cleaved with a restriction enzyme, and then subsequently ligated to a pair of complementary oligonucleotides containing the TA or CAR variant mutation. The restriction fragments of the plasmid and oligonucleotide can be ligated to one another.

In other embodiments, another technique used to generate the variant TA or CAR sequence is PCR site directed mutagenesis. Mutagenic oligonucleotide primers are used to introduce the desired mutation and to provide a PCR fragment carrying the mutated sequence. Additional oligonucleotides may be used to extend the ends of the mutated fragment to provide restriction sites suitable for restriction enzyme digestion and insertion into the gene.

Commercial kits for site-directed mutagenesis techniques are also available. For example, the Quikchange™ kit uses complementary mutagenic primers to PCR amplify a gene region using a high-fidelity non-strand-displacing DNA polymerase such as pfu polymerase. The reaction generates a nicked, circular DNA which is relaxed. The template DNA is eliminated by enzymatic digestion with a restriction enzyme such as DpnI which is specific for methylated DNA.

In some embodiments, optimization method is directed evolution. Directed evolution is a powerful apprTAh that involves the introduction of mutations targeted to a specific gene to improve and/or alter the properties of an enzyme. Improved and/or altered enzymes can be identified through the development and implementation of sensitive high-throughput screening assays that allow the automated screening of many enzyme variants (for example, >104). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened. Numerous directed evolution technologies have been developed (for reviews, see Hibbert et al., Biomol. Eng 22:11-19 (2005); Huisman and Lalonde, In Biocatalysis in the pharmaceutical and biotechnology industries pgs. 717-742 (2007), Patel (ed.), CRC Press; Otten and Quax. Biomol. Eng 22:1-9 (2005); and Sen et al., Appl Biochem. Biotechnol 143:212-223 (2007)) to be effective at creating diverse variant libraries, and these methods have been successfully applied to the improvement of a wide range of properties across many enzyme classes. Enzyme characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Km), including broadening substrate binding to include non-natural substrates; inhibition (Ki), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well-known to those skilled in the art. Any of these can be used to alter and/or optimize the activity of a 6ACA, hexamethylenediamine, caprolactam or 1,6-hexanediol pathway enzyme or protein. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e17 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional ts mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

Any of the aforementioned methods for mutagenesis can be used alone or in any combination. Additionally, any one or combination of the directed evolution methods can be used in conjunction with adaptive evolution techniques, as described herein.

A cell having the desired enzymatic activity can be identified using any method known in the art. For example, enzyme activity assays can be used to identify cells having enzyme activity, see, for example, Enzyme Nomenclature, Academic Press, Inc., New York 2007. Other assays that can be used to determine reaction of TA on adipate semialdhyde, CAR on 6ACA and/or TA2 on 6-aminocaproate semialdehyde include GC/MS analysis. In other examples, levels of NADH/NADPH can be monitored. For example, the NADH/NADPH can be monitored colorimetrically or spectroscopically using NADP/NADPH assay kits (e.g. ab65349 available from ABCAM™).

The disclosed TA enzyme can be used in pathways for the production of nylon intermediates. In some embodiments, a non-naturally occurring microorganism can be used in the production of adipate semialdehyde or other nylon intermediates that are produced using the adipate semialdehyde as an intermediate. One exemplary intermediate using adipate semialdehyde as a substrate for a TA enzyme described herein is 6ACA.

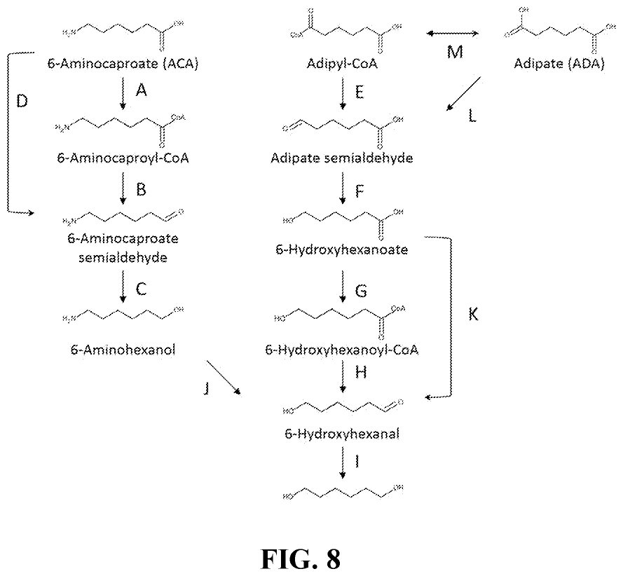

The disclosed CAR enzyme can be used in pathways for the production of nylon intermediates and/or for the production 1,6-hexanediol intermediates. In some embodiments, a non-naturally occurring microorganism can be used in the production of 6ACA or other nylon intermediate, or a 1,6-hexandiol intermediate that are produced using the 6ACA as an intermediate. One exemplary intermediate for both nylon and 1,6-hexanediol using 6ACA as a substrate for a CAR enzyme described herein is 6-aminocaproate semialdehyde. Accordingly, certain nylon intermediates can also be 1,6-hexanediol intermediates (see, e.g., FIG. 8) and, unless otherwise stated, are referred to herein as nylon intermediates.

The disclosed TA2 enzyme can be used in pathways for the production of nylon intermediates. In some embodiments, a non-naturally occurring microorganism can be used in the production of 6-aminocaproate semialdehyde or other nylon intermediates that are produced using the 6-aminocaproate semialdehyde as an intermediate. One exemplary intermediate using 6-aminocaproate semialdehyde as a substrate for a TA2 enzyme described herein is hexamethylenediamine.

In some embodiments, genetically modified cells (e. g. non-naturally occurring microorganisms) are capable of producing the nylon intermediates such as 6-aminocaproic acid, caprolactam, and hexamethylenediamine.

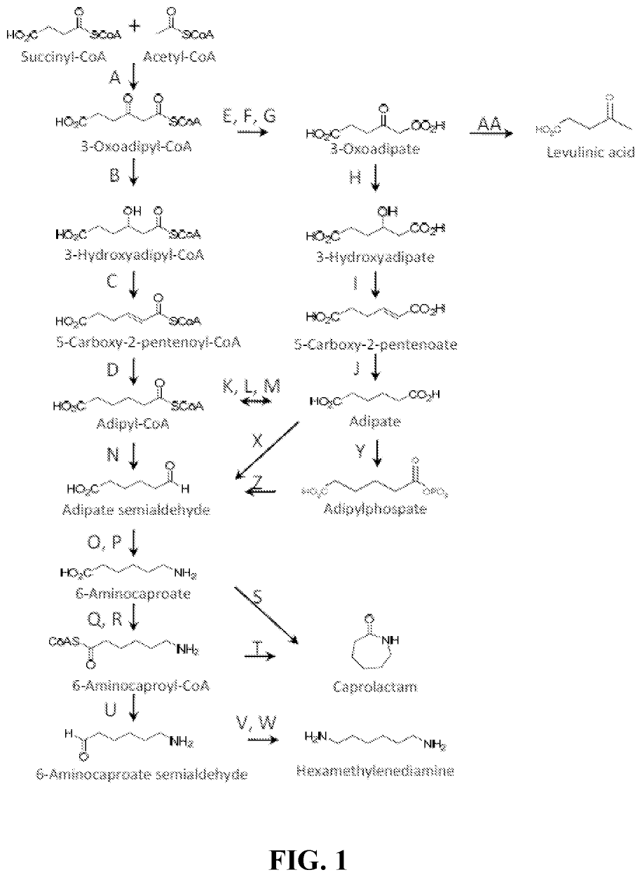

In some embodiments, the nylon intermediates are biosynthesized using the pathway described in FIG. 1. In some embodiments, FIG. 1 pathway is provided in genetically modified cell described herein (e. g., a non-naturally occurring microorganism) where the pathway includes at least one exogenous nucleic acid encoding a pathway enzyme expressed in a sufficient amount to produce 6-aminocaproic acid, caprolactam, and hexamethylenediamine.

In some embodiments the pathway is an HMD pathway as set forth in FIG. 1. The HMD pathway is provided in genetically modified cell described herein (e. g., a non-naturally occurring microorganism) where the HMD pathway includes at least one exogenous nucleic acid encoding a HMD pathway enzyme expressed in a sufficient amount to produce HMD. The enzymes are 1A is a 3-oxoadipyl-CoA thiolase; 1B is a 3-oxoadipyl-CoA reductransaminasee; 1C is a 3-hydroxyadipyl-CoA dehydratransaminasee; 1D is aadipate semialdehydereductransaminasee; 1E is a 3-oxoadipyl-CoA/acyl-CoA transferase; 1F is a 3-oxoadipyl-CoA synthase; 1G is a 3-oxoadipyl-CoA hydrolase; 1H is a 3-oxoadipate reductransaminasee; 1I is a 3-hydroxyadipate dehydratransaminasee; 1J is a 5-carboxy-2-pentenoate reductransaminasee; 1K is an adipyl-CoA/acyl-CoA transferase; 1L is an adipyl-CoA synthase; 1M is an adipyl-CoA hydrolase; 1N is an adipyl-CoA reductransaminasee (aldehyde forming); 1O is a 6-aminocaproate transaminase; 1P is a 6-aminocaproate dehydrogenase; 1Q is a 6-aminocaproyl-CoA/acyl-CoA transferase; 1R is a 6-aminocaproyl-CoA synthase; 1S is an amidohydrolase; 1T is spontaneous cyclization; 1U is a 6-aminocaproyl-CoA reductransaminasee (aldehyde forming); 1V is a HMID transaminase; and 1W is a HMD dehydrogenase.

With reference to FIG. 1, in some embodiments, the non-naturally occurring microorganism has one or more of the following pathways: ABCDNOPQRUVW; ABCDNOPQRT; or: ABCDNOPS. Other exemplary pathways that include the TA enzyme to produce adipate semialdehyde include those described in U.S. Pat. No. 8,377,680 incorporated herein by reference in its entirety.

FIG. 1 also shows a pathway from 6-aminocaproate to 6-aminocaproyl-CoA by a transferase or synthase enzyme (FIG. 1, Step Q or R) followed by the spontaneous cyclization of 6-aminocaproyl-CoA to form caprolactam (FIG. 1, Step T). In other embodiments, 6-aminocaproate is activated to 6-aminocaproyl-CoA (FIG. 1, Step Q or R), followed by a reduction (FIG. 1, Step U) and amination (FIG. 1, Step V or W) to form HMD. 6-Aminocaproic acid can also be activated to 6-aminocaproyl-phosphate instead of 6-aminocaproyl-CoA. 6-Aminocaproyl-phosphate can spontaneously cyclize to form caprolactam. In some embodiments, 6-aminocaproyl-phosphate can be reduced to 6-aminocaproate semialdehyde, which can be then converted to HMD as depicted in FIG. 1.

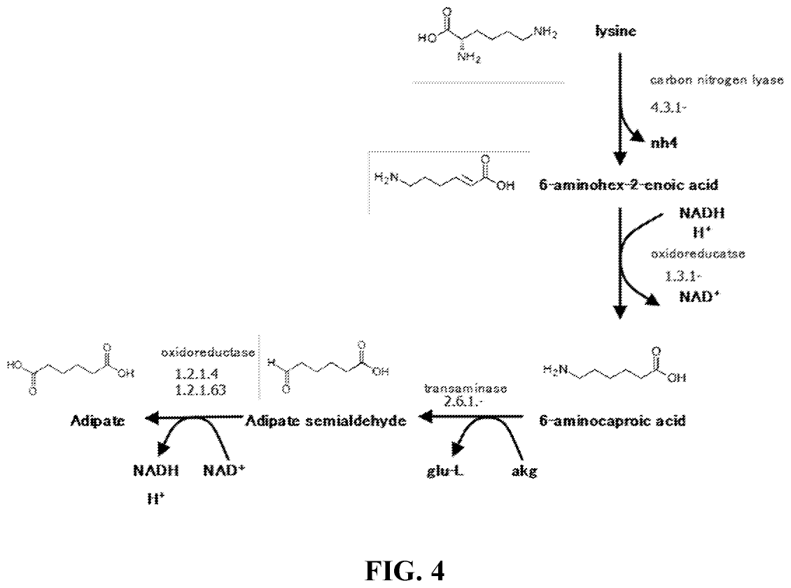

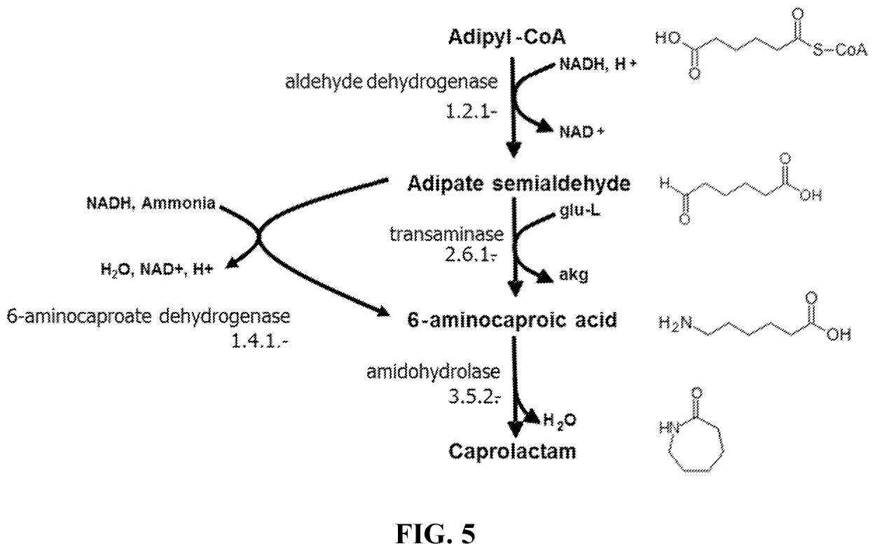

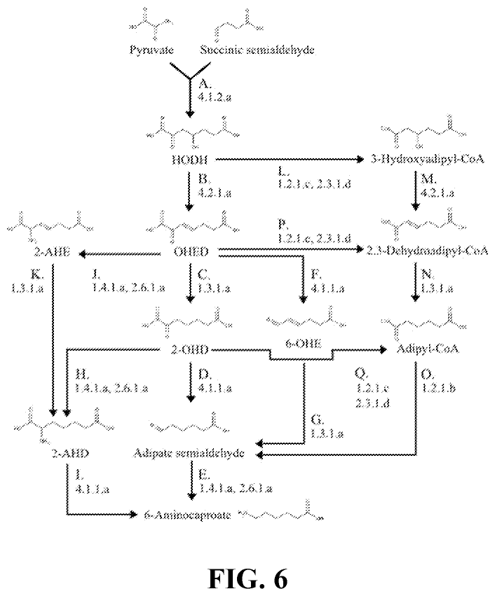

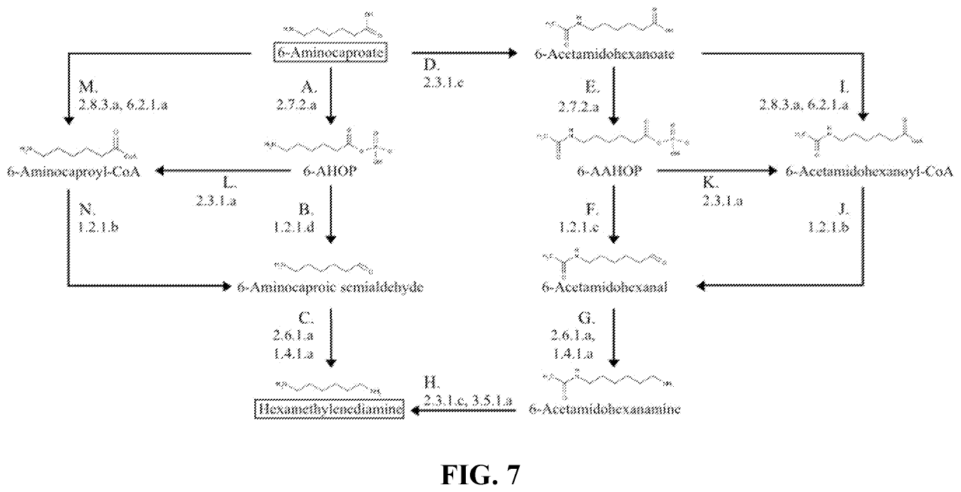

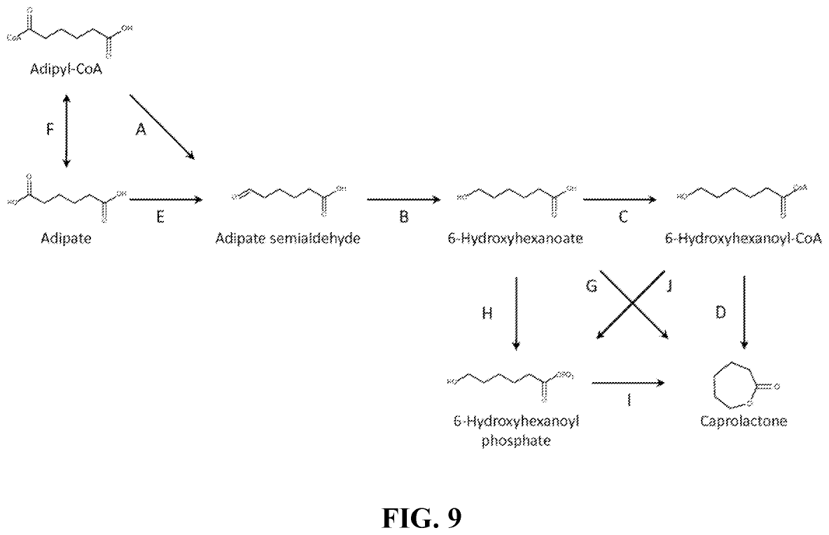

In some embodiments, the non-naturally occurring microrganisms can generate adipate, 6ACA, caprolactone, hexamethyelenediamine or caproclactam as shown in the pathways of FIG. 4-10.

In some embodiments, the non-naturally occurring microrganisms can generate 1,6-hexandiol. FIG. 8 exemplifies biosynthetic pathways to 1,6-hexanediol through nylon intermediates such as 6ACA, 6-aminocaproyl-CoA, 6-aminocaproate semialdehyde, adipate, adipyl-CoA and aidpate semialdehyde.

In some embodiments, the non-naturally occurring microbial organisms further include an exogenously expressed nucleic acid encoding an aldehyde dehydrognease (ALD) or a transenoyl reductase (TER) or both. The ALD reacts with adipyl-CoA to produce adipate semialdehyde, whereas the TER reacts with 5-carboxy-2-pentenoyl-CoA (CPCoA) to form adipylCoA.

In some embodiments, the ALD enzymes have greater catalytic efficiency and activity for the adipyl CoA substrate as compared to succinyl-CoA, or acetyl-CoA, or both substrates. In some embodiments, the ALD enzymes are as shown below in Table 4. In some embodiments, the TER enzyme sequences that can be exogenously expressed from an encoding nucleic acid in a non-naturally occurring microorganism of the disclosure are as shown below in Table 5.

In some embodiments, the TA enzyme sequences that can be exogenously expressed from an encoding nucleic acid in a non-naturally occurring microorganism of the disclosure are shown below in Table 6. In some embodiments, the TA enzyme variant sequences that can be exogenously expressed from an encoding nucleic acid in a non-naturally occurring microorganism of the are shown below in Table 7.

In some embodiments, the CAR enzyme sequences that can be exogenously expressed from an encoding nucleic acid in a non-naturally occurring microorganism of the disclosure are shown below in Table 8. In some embodiments, the CAR variant sequences that can be exogenously expressed from an encoding nucleic acid in a non-naturally occurring microorganism of the disclosure are shown below in Table 9.

In some embodiments, the CAR enzyme sequences that can be exogenously expressed from an encoding nucleic acid in a non-naturally occurring microorganism of the disclosure comprises an amino acid sequence of SEQ ID NO: 153 and one or more amino acid alterations shown below in Table 10. In some embodiments, the CAR enzyme can comprise 1, 2, 3, 4, 5, 6, 7, or 8 amino acid alterations shown below in Table 10.

In some embodiments, the TA2 enzyme sequences that can be exogenously expressed from an encoding nucleic acid in a non-naturally occurring microorganism of the disclosure are shown below in Table 11.

Any, some or all of the TA, CAR, TA2, TER and/or ALD enzymes described herein can be used in any of the biosynthetic pathways described herein so long as the substrate and products of the referenced TA, CAR, TA2, TER and/or ALD enzymatic conversion or conversions are intermediates within the described pathway. For example, a TER can be substituted into any pathway described herein for the referenced enzyme having a conversion of 5-carboxy-2-pentenoyl-CoA (also referred to as 2,3-dehydroadipyl-CoA) to adipyl-CoA. Similarly, an ALD can be substituted into any pathway described herein for the referenced enzyme having a conversion of adipyl-CoA to adipate semialdehyde. Further, a TA enzyme can be substituted into any pathway described herein for the referenced enzyme having a conversion of adipate semialdehyde to 6ACA. Still further, CAR enzyme can be substituted into any pathway described herein for the referenced enzyme having a conversion of 6ACA to 6-aminocaproate semialdehyde. Additionally, TA2 enzyme can be substituted into any pathway described herein for the referenced enzyme having a conversion of 6-aminocaproate to HMD. Accordingly, any combination and/or permutation of any one, two, three, four or all five of TA, CAR, TA2, TER and/or ALD can be utilized in a biosynthetic pathway described herein.

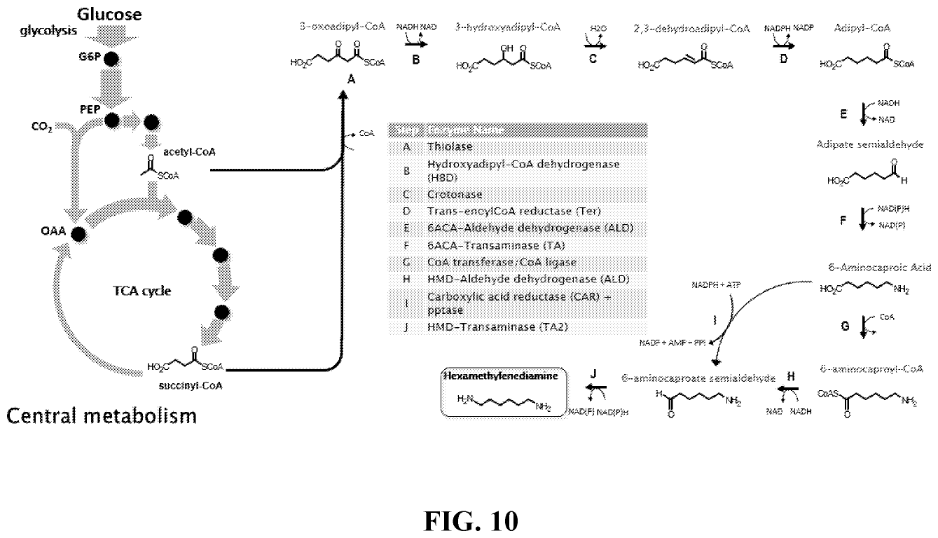

One exemplary pathway that can utilize any, some or all of TA, CAR, TA2, TER and/or ALD is represented in FIG. 10 by reference to one specific embodiment where all of TA, CAR, TA2, TER and ALD are utilized. In some embodiments, the nylon intermediates are biosynthesized using the pathway described in FIG. 10. In some embodiments, FIG. 10 pathway is provided in genetically modified cell described herein (e.g., a non-naturally occurring microorganism) where the pathway includes at least one exogenous nucleic acid encoding a pathway enzyme expressed in a sufficient amount to produce 6-aminocaproic acid, 6-aminoaproate semialdehyde and hexamethylenediamine.

In some embodiments the pathway is an HMD pathway as set forth in FIG. 10. The HMD pathway is provided in genetically modified cell described herein (e. g., a non-naturally occurring microorganism) where the HMD pathway includes at least one exogenous nucleic acid encoding a HMD pathway enzyme expressed in a sufficient amount to produce HMD. Starting from succinyl-CoA and acetyl-CoA the enzymes are designated are: (A) thiolase; (B) hydroxyadipyl-CoA dehydrogenase (HBD); (C) crotonase; (D) trans-enoyl-CoA reductase (Ter); (E) 6ACA-aldehyde dehydrogenase (ALD); (F) 6ACA-transaminase (TA); (G) CoA transferase/CoA ligase; (H) HMD-aldehyde dehydrogenase (ALD); (I) carboxylic acid reductase (CAR), and (J) HMD-transaminase (TA2). An exogenous nucleic acid encoding phosphopantetheinyl transferase (PPTase) can additionally be included. Employing a CAR enzyme, this pathway can omit steps G and H. Without using CAR, step I can be omitted.

With reference to FIG. 10 and with utilization of a CAR enzyme described herein, the non-naturally occurring microorganism has the following HMD pathway: ABCDEFIJ where step I is the CAR conversion of 6ACA to 6-amainocaproate. Enzymes D, E, F and J for the above pathway correspond to the TER, ALD, TA and TA2 enzymes, respectfully.

As used herein, the term “non-naturally occurring” when used in reference to a microbial organism or microorganism is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes within a 6-aminocaproic acid, caprolactam, hexamethylenediamine or 1,6-hexanediol biosynthetic pathway described herein.

A metabolic modification refers to a biochemical reaction that is altered from its naturally occurring state. Therefore, non-naturally occurring microorganisms can have genetic modifications to nucleic acids encoding metabolic polypeptides or, functional fragments thereof. Exemplary metabolic modifications are disclosed herein.

As used herein, the terms “microbial,” “microbial organism” or “microorganism” has been used interchangeably and is intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, “adipate,” having the chemical formula —OOC—(CH₂)₄—COO— (see FIG. 1) (IUPAC name hexanedioate), is the ionized form of adipic acid (IUPAC name hexanedioic acid), and it is understood that adipate and adipic acid can be used interchangeably throughout to refer to the compound in any of its neutral or ionized forms, including any salt forms thereof. It is understood by those skilled understand that the specific form will depend on the pH.

As used herein, “6-aminocaproate,” having the chemical formula —OOC—CH₂)₅—NH₂ (see FIG. 1, and abbreviated as 6-ACA), is the ionized form of 6-aminocaproic acid (IUPAC name 6-aminohexanoic acid), and it is understood that 6-aminocaproate and 6-aminocaproic acid can be used interchangeably throughout to refer to the compound in any of its neutral or ionized forms, including any salt forms thereof. It is understood by those skilled understand that the specific form will depend on the pH.

As used herein, “caprolactam” (IUPAC name azepan-2-one) is a lactam of 6-aminohexanoic acid (see FIG. 1, and abbreviated as CPO).

As used herein, “hexamethylenediamine,” also referred to as 1,6-diaminohexane or 1,6-hexanediamine, has the chemical formula H2N(CH2)6NH2 (see FIG. 1 and abbreviated as HMD).

As used herein, “1,6-hexanediol,” also referred to as hexane-1,6-diol and hexamethylenediol, has the chemical structure C₆H₁₄O₂ (see FIG. 8 and abbreviated as HDO).

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

As used herein, the term “osmoprotectant” when used in reference to a culture or growth condition is intended to mean a compound that acts as an osmolyte and helps a microbial organism as described herein survive osmotic stress. Osmoprotectants include, for example, betaines, amino acids, and the sugar trehalose. Non-limiting examples of such are glycine betaine, praline betaine, dimethylthetin, dimethylslfonioproprionate, 3-dimethylsulfonio-2-methylproprionate, pipecolic acid, dimethylsulfonioacetate, choline, L-carnitine and ectoine.

As used herein, the term “growth-coupled” when used in reference to the production of a biochemical is intended to mean that the biosynthesis of the referenced biochemical is produced during the growth phase of a microorganism. In a particular embodiment, the growth-coupled production can be obligatory, meaning that the biosynthesis of the referenced biochemical is an obligatory product produced during the growth phase of a microorganism.

As used herein, “metabolic modification” is intended to refer to a biochemical reaction that is altered from its naturally occurring state. Metabolic modifications can include, for example, elimination of a biochemical reaction activity by functional disruptions of one or more genes encoding an enzyme participating in the reaction.

As used herein, the term “gene disruption,” or grammatical equivalents thereof, is intended to mean a genetic alteration that renders the encoded gene product inactive. The genetic alteration can be, for example, deletion of the entire gene, deletion of a regulatory sequence required for transcription or translation, deletion of a portion of the gene which results in a truncated gene product, or by any of various mutation strategies that inactivate the encoded gene product. One particularly useful method of gene disruption is complete gene deletion because it reduces or eliminates the occurrence of genetic reversions in the non-naturally occurring microorganisms.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism.

The term “heterologous” refers to a molecule, material, or activity derived from a source other than the referenced species whereas “homologous” refers to a molecule, material, or activity derived from the host microbial organism. Accordingly, exogenous expression of an encoding nucleic acid can utilize either or both a heterologous or homologous encoding nucleic acid.

As used herein the term “about” means±10% of the stated value. The term “about” can mean rounded to the nearest significant digit. Thus, about 5% means 4.5% to 5.5%. Additionally, about in reference to a specific number also includes that exact number. For example, about 5% also includes exact 5%.

As used herein the term “bioderived” in the context of 6-aminocaproic acid, 1,6-hexanediol, caprolactone, caprolactam, hexamethylenediamine or 1,6-hexanediol means that these compounds are synthesized in a microbial organism.

It is understood that when more than one exogenous nucleic acid is included in a microbial organism, the exogenous nucleic acids refer to the referenced encoding nucleic acid or biosynthetic activity, as exemplified above or below. It is further understood, as disclosed herein, that such exogenous nucleic acids can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, which are not integrated into the host chromosome, and the plasmids remain as extra-chromosomal elements, and still be considered as two or more exogenous nucleic acids. The number of referenced exogenous nucleic acids or biosynthetic activities refers to the number of encoding nucleic acids or the number of biosynthetic activities, not the number of separate nucleic acids introduced into the host organism.

The non-naturally occurring microbial organisms can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

In the case of gene disruptions, a particularly useful stable genetic alteration is a gene deletion. The use of a gene deletion to introduce a stable genetic alteration is particularly useful to reduce the likelihood of a reversion to a phenotype prior to the genetic alteration. For example, stable growth-coupled production of a biochemical can be achieved, for example, by deletion of a gene encoding an enzyme catalyzing one or more reactions within a set of metabolic modifications. The stability of growth-coupled production of a biochemical can be further enhanced through multiple deletions, significantly reducing the likelihood of multiple compensatory reversions occurring for each disrupted activity.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less than 25% can also be considered to have arisen by vertical descent if their three-dimensional structure also shows similarities. Members of the serine protease family of enzymes, including tissue plasminogen activator and elastransaminasee, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastransaminasee proteolysis and plasminogen proteolysis, two types of serine protease activity, into distinct molecules as plasminogen activator and elastransaminasee. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms having 6-aminocaproic acid, caprolactam, hexamethylenediamine or 1,6-hexanediol biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes. In gene disruption strategies, evolutionally related genes can also be disrupted or deleted in a host microbial organism, paralogs or orthologs, to reduce or eliminate activities to ensure that any functional redundancy in enzymatic activities targeted for disruption do not short circuit the designed metabolic modifications.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary paramemeters for determining relatedness of two or more sequences using the BLAST algorithm, for example, can be as set forth below. Briefly, amino acid sequence alignments can be performed using BLASTP version 2. 2. 29+ (Jan. 14, 2014) and the following parameTransaminase: Matrix: 0 BLOSUM62; gap open: 11; gap extension: 1; x_dropoff: 50; expect: 10. 0; wordsize: 3; filter: on. Nucleic acid sequence alignments can be performed using BLASTN version 2. 0. 6 (Sep. 16, 1998) and the following parameTransaminase: Match: 1; mismatch: −2; gap open: 5; gap extension: 2; x_dropoff: 50; expect: 10. 0; wordsize: 11; filter: off. Those skilled in the art will know what modifications can be made to the above parameTransaminase to either increase or decrease the stringency of the comparison, for example, and determine the relatedness of two or more sequences.

It is understood that any of the pathways disclosed herein, including those as described in the Figures can be used to generate a non-naturally occurring microbial organism that produces any pathway intermediate or product, as desired. As disclosed herein, such a microbial organism that produces an intermediate can be used in combination with another microbial organism expressing downstream pathway enzymes to produce a desired product. However, it is understood that a non-naturally occurring microbial organism that produces a 6-aminocaproic acid, caprolactam, hexamethylenediamine or 1,6-hexanediol pathway intermediate can be utilized to produce the intermediate as a desired product.

Described herein with general reference to the metabolic reaction, reactant or product thereof, or with specific reference to one or more nucleic acids or genes encoding an enzyme associated with or catalyzing the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze the referenced reaction, reactant or product. Likewise, given the well-known fields of metabolic biochemistry, enzymology and genomics, reference herein to a gene or encoding nucleic acid also constitutes a reference to the corresponding encoded enzyme and the reaction it catalyzes as well as the reactants and products of the reaction.

The non-naturally occurring microbial organisms can be produced by introducing expressible nucleic acids encoding one or more of the enzymes participating in one or more 6-aminocaproic acid, caprolactam, hexamethylenediamine or 1,6-hexanediol biosynthetic pathways. Depending on the host microbial organism chosen for biosynthesis, nucleic acids for some or all of a particular 6-aminocaproic acid, caprolactam, hexamethylenediamine or 1,6-hexanediol biosynthetic pathway can be expressed. For example, if a chosen host is deficient in one or more enzymes for a desired biosynthetic pathway, then expressible nucleic acids for the deficient enzyme(s) are introduced into the host for subsequent exogenous expression. Alternatively, if the chosen host exhibits endogenous expression of some pathway genes, but is deficient in others, then an encoding nucleic acid is needed for the deficient enzyme(s) to achieve 6-aminocaproic acid, caprolactam, hexamethylenediamine or 1,6-hexanediol biosynthesis. Thus, a non-naturally occurring microbial organism can be produced by introducing exogenous enzyme activities to obtain a desired biosynthetic pathway or a desired biosynthetic pathway can be obtained by introducing one or more exogenous enzyme activities that, together with one or more endogenous enzymes, produce a desired product such as 6-aminocaproic acid, caprolactam, hexamethylenediamine or 1,6-hexanediol.

Depending on the 6-aminocaproic acid, caprolactam, hexamethylenediamine or 1,6-hexanediol biosynthetic pathway constituents of a selected host microbial organism, the non-naturally occurring microbial organisms will include at least one exogenously expressed 6-aminocaproic acid, caprolactam, hexamethylenediamine or 1,6-hexanediol pathway-encoding nucleic acid and up to all encoding nucleic acids for one or more adipate, 6-aminocaproic acid or caprolactam biosynthetic pathways. For example, 6-aminocaproic acid, caprolactam, hexamethylenediamine or 1,6-hexanediol biosynthesis can be established in a host deficient in a pathway enzyme through exogenous expression of the corresponding encoding nucleic acid. In a host deficient in all enzymes of a 6-aminocaproic acid, caprolactam, hexamethylenediamine or 1,6-hexanediol pathway, exogenous expression of all enzymes in the pathway can be included, although it is understood that all enzymes of a pathway can be expressed even if the host contains at least one of the pathway enzymes.

Given the teachings and guidance provided herein, those skilled in the art will understand that the number of encoding nucleic acids to introduce in an expressible form will, at least, parallel the adipate, 6-aminocaproic acid, caprolactam, hexamethylenediamine or 1,6-hexanediol pathway deficiencies of the selected host microbial organism. Therefore, a non-naturally occurring microbial organism can have at least one, two, three, four, five, six, seven, eight, nine, ten, eleven or twelve, up to all nucleic acids encoding the above enzymes constituting a 6-aminocaproic acid, caprolactam, hexamethylenediamine or 1,6-hexanediol biosynthetic pathway. In some embodiments, the non-naturally occurring microbial organisms also can include other genetic modifications that facilitate or optimize 6-aminocaproic acid, caprolactam, hexamethylenediamine or 1,6-hexanediol biosynthesis or that confer other useful functions onto the host microbial organism. One such other functionality can include, for example, augmentation of the synthesis of one or more of the 6-aminocaproic acid, caprolactam, hexamethylenediamine or 1,6-hexanediol pathway precursors such as succinyl-CoA and/or acetyl-CoA in the case of adipate synthesis, or adipyl-CoA or adipate in the case of 6-aminocaproic acid, caprolactam or HMID synthesis, including the adipate pathway enzymes disclosed herein, or pyruvate and succinic semialdehyde, glutamate, glutaryl-CoA, homolysine or 2-amino-7-oxosubarate in the case of 6-aminocaprioate synthesis, or 6-aminocaproate, glutamate, glutaryl-CoA, pyruvate and 4-aminobutanal, or 2-amino-7-oxosubarate in the case of hexamethylenediamine synthesis.

In some embodiments, a non-naturally occurring microbial organism has at least one exogenous nucleic acid encoding a transaminase that reacts with adipate semialdhyde to form 6ACA and selected from transaminases comprising the amino acid sequences having at least about 50% amino acid sequence identity to at least 25, 50, 75, 100, 150, 200, 250, 300, or more contiguous amino acids of any of any of SEQ ID NOs: 1, 3, 4, 5, 9, 12, 13, 26, 27, 30, 31, 38, 50, 52, 64, 74, 78, 79, 81, 91, 106, 108, and 116.

In some embodiments, a non-naturally occurring microbial organism has at least one exogenous nucleic acid encoding a CAR that reacts with 6ACA to form 6-aminocaproate semialdehyde and selected from CARs comprising the amino acid sequences having at least about 50% sequence identity to at least 25, 50, 75, 100, 150, 200, 250, 300, or more contiguous amino acids of any of SEQ ID NOS:150-165, 168-171, 173-178, 180, 183-185, 187-188, 190-193, 195, 198-200, 202-216, 218-219, 221-230, 232-238, 241-244, 246-249, 251-252 and 255-264.

In some embodiments, a non-naturally occurring microbial organism has at least one exogenous nucleic acid encoding a CAR that reacts with 6ACA to form 6-aminocaproate semialdehyde and selected from CARs comprising the amino acid sequences having at least about 50% sequence identity to at least 25, 50, 75, 100, 150, 200, 250, 300, or more contiguous amino acids of any of SEQ ID NOS: 265 and 267-296.

Generally, a host microbial organism is selected such that it produces the precursor of a 6-aminocaproic acid, caprolactam, hexamethylenediamine or 1,6-hexanediol pathway, either as a naturally produced molecule or as an engineered product that either provides de novo production of a desired precursor or increased production of a precursor naturally produced by the host microbial organism. A host organism can be engineered to increase production of a precursor, as disclosed herein. In addition, a microbial organism that has been engineered to produce a desired precursor can be used as a host organism and further engineered to express enzymes or proteins of a 6-aminocaproic acid, caprolactam, hexamethylenediamine or 1,6-hexanediol pathway.

In some embodiments, a non-naturally occurring microbial organism is generated from a host that contains the enzymatic capability to synthesize 6-aminocaproic acid, caprolactam, hexamethylenediamine or 1,6-hexanediol. In this specific embodiment it can be useful to increase the synthesis or accumulation of a 6-aminocaproic acid, caprolactam, hexamethylenediamine or 1,6-hexanediol pathway product to, for example, drive 6-aminocaproic acid, caprolactam, hexamethylenediamine or 1,6-hexanediol pathway reactions toward 6-aminocaproic acid, caprolactam, hexamethylenediamine or 1,6-hexanediol production. Increased synthesis or accumulation can be accomplished by, for example, overexpression of nucleic acids encoding one or more of the above-described 6-aminocaproic acid, caprolactam, hexamethylenediamine or 1,6-hexanediol pathway enzymes. Over expression of the 6-aminocaproic acid, caprolactam, hexamethylenediamine or 1,6-hexanediol pathway enzyme or enzymes can occur, for example, through exogenous expression of the endogenous gene or genes, or through exogenous expression of the heterologous gene or genes. Therefore, naturally occurring organisms can be readily generated to be non-naturally occurring microbial organisms, for example, producing 6-aminocaproic acid, caprolactam, hexamethylenediamine or 1,6-hexanediol, through overexpression of at least one, two, three, four, five, six, seven, eight, nine, ten, eleven, twelve, thirteen, fourteen, that is, up to all nucleic acids encoding 6-aminocaproic acid, caprolactam, hexamethylenediamine or 1,6-hexanediol biosynthetic pathway enzymes. In addition, a non-naturally occurring organism can be generated by mutagenesis of an endogenous gene that results in an increase in activity of an enzyme in the 6-aminocaproic acid, caprolactam, hexamethylenediamine or 1,6-hexanediol biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

In some embodiments, a non-naturally occurring microbial organism includes one or more gene disruptions, where the organism produces a 6-ACA, adipate and/or HMD. The disruptions occur in genes encoding an enzyme that couples production of adipate, 6-ACA and/or HMD to growth of the organism when the gene disruption reduces the activity of the enzyme, such that the gene disruptions confer increased production of adipate, 6-ACA and/or HMD onto the non-naturally occurring organism. Thus, in some embodiments is provided a non-naturally occurring microbial organism, comprising one or more gene disruptions, the one or more gene disruptions occurring in genes encoding proteins or enzymes wherein the one or more gene disruptions confer increased production of adipate, 6-ACA and/or HMD in the organism. As disclosed herein, such an organism contains a pathway for production of adipate, 6-ACA and/or HMD.

It is understood that, in methods, any of the one or more exogenous nucleic acids can be introduced into a microbial organism to produce a non-naturally occurring microbial organism. The nucleic acids can be introduced so as to confer, for example, a 6-aminocaproic acid, caprolactam, hexamethylenediamine or 1,6-hexanediol biosynthetic pathway onto the microbial organism. Alternatively, encoding nucleic acids can be introduced to produce an intermediate microbial organism having the biosynthetic capability to catalyze some of the required reactions to confer 6-aminocaproic acid, caprolactam, hexamethylenediamine or 1,6-hexanediol biosynthetic capability. For example, a non-naturally occurring microbial organism having a 6-aminocaproic acid, caprolactam, hexamethylenediamine or 1,6-hexanediol biosynthetic pathway can comprise at least two exogenous nucleic acids encoding desired enzymes. In the case of adipate production, at least two exogenous nucleic acids can encode the enzymes such as the combination of succinyl-CoA: acetyl-CoA acyl transferase and 3-hydroxyacyl-CoA dehydrogenase, or succinyl-CoA: acetyl-CoA acyl transferase and 3-hydroxyadipyl-CoA dehydratransaminasee, or 3-hydroxyadipyl-CoA and adipate semialdehyde transaminase, or 3-hydroxyacyl-CoA and adipyl-CoA synthetase, and the like. In the case of caprolactam production, at least two exogenous nucleic acids can encode the enzymes such as the combination of CoA-dependent trans-enoyl-CoA reductase and transaminase, or CoA-dependent trans-enoyl-CoA reductransaminasee and amidohydrolase, or transaminase and amidohydrolase. In the case of 6-aminocaproic acid production, at least two exogenous nucleic acids can encode the enzymes such as the combination of an 4-hydroxy-2-oxoheptane-1,7-dioate (HODH) TAolase and a 2-oxohept-4-ene-1,7-dioate (OHED) hydratransaminasee, or a 2-oxohept-4-ene-1,7-dioate (OHED) hydratransaminasee and a 2-aminoheptane-1,7-dioate (2-AHD) decarboxylase, a 3-hydroxyadipyl-CoA dehydratransaminasee and a adipyl-CoA dehydrogenase, a glutamyl-CoA transferase and a 6-aminopimeloyl-CoA hydrolase, or a glutaryl-CoA beta-ketothiolase and a 3-aminopimelate 2,3-aminomutransaminasee. In the case of hexamethylenediamine production, at least two exogenous nucleic acids can encode the enzymes such as the combination of 6-aminocaproate kinase and [(6-aminohexanoyl)oxy]phosphonate (6-AHOP) oxidoreductransaminasee, or a 6-acetamidohexanoate kinase and an [(6-acetamidohexanoyl)oxy]phosphonate (6-AAHOP) oxidoreductransaminasee, 6-aminocaproate N-acetyltransferase and 6-acetamidohexanoyl-CoA oxidoreductransaminasee, a 3-hydroxy-6-aminopimeloyl-CoA dehydratransaminasee and a 2-amino-7-oxoheptanoate aminotransferase, or a 3-oxopimeloyl-CoA ligase and a homolysine decarboxylase. Thus, it is understood that any combination of two or more enzymes of a biosynthetic pathway can be included in a non-naturally occurring microbial organism. In the case of 6ACA, 6-aminocaproate semialdehyde or HMD, by reference to FIG. 10, at least two exogenous nucleic acids can encode the enzymes such as the combination of enzymes represented by steps 10A and 10B, 10B and 10C, 10C and 10D, 10D and 10E, 10E and 10F, 10F and 10I and/or 10I and 10J, or any combination thereof of two, three, four, five, six, seven and/or eight of the enzymes represented by steps 10A, 10B, 10C, 10D, 10E, 10F, 10I and/or 10J.

Similarly, it is understood that any combination of three or more enzymes of a biosynthetic pathway can be included in a non-naturally occurring microbial organism, for example, in the case of adipate production, the combination of enzymes succinyl-CoA: acetyl-CoA acyl transferase, 3-hydroxyacyl-CoA dehydrogenase, and 3-hydroxyadipyl-CoA dehydratransaminasee; or succinyl-CoA: acetyl-CoA acyl transferase, 3-hydroxyacyl-CoA dehydrogenase andadipate semialdehydereductransaminasee; or succinyl-CoA: acetyl-CoA acyl transferase, 3-hydroxyacyl-CoA dehydrogenase and adipyl-CoA synthetransaminasee; or 3-hydroxyacyl-CoA dehydrogenase, 3-hydroxyadipyl-CoA dehydratransaminasee and adipyl-CoA: acetyl-CoA transferase, and so forth, as desired, so long as the combination of enzymes of the desired biosynthetic pathway results in production of the corresponding desired product. In the case of 6-aminocaproic acid production, the at least three exogenous nucleic acids can encode the enzymes such as the combination of an 4-hydroxy-2-oxoheptane-1,7-dioate (HODH) TAolase, a 2-oxohept-4-ene-1,7-dioate (OHED) hydratransaminasee and a 2-oxoheptane-1,7-dioate (2-OHD) decarboxylase, or a 2-oxohept-4-ene-1,7-dioate (OHED) hydratransaminasee, a 2-aminohept-4-ene-1,7-dioate (2-AHE) reductransaminasee and a 2-aminoheptane-1,7-dioate (2-AHD) decarboxylase, or a 3-hydroxyadipyl-CoA dehydratransaminasee, 2,3-dehydroadipyl-CoA reductransaminasee and a adipyl-CoA dehydrogenase, or a 6-amino-7-carboxyhept-2-enoyl-CoA reductransaminasee, a 6-aminopimeloyl-CoA hydrolase and a 2-aminopimelate decarboxylase, or a glutaryl-CoA beta-ketothiolase, a 3-aminating oxidoreductransaminasee and a 2-aminopimelate decarboxylase, or a 3-oxoadipyl-CoA thiolase, a 5-carboxy-2-pentenoate reductransaminasee and a adipate reductransaminasee. In the case of hexamethylenediamine production, at least three exogenous nucleic acids can encode the enzymes such as the combination of 6-aminocaproate kinase, [(6-aminohexanoyl)oxy]phosphonate (6-AHOP) oxidoreductransaminasee and 6-aminocaproic semialdehyde aminotransferase, or a 6-aminocaproate N-acetyltransferase, a 6-acetamidohexanoate kinase and an [(6-acetamidohexanoyl)oxy]phosphonate (6-AAHOP) oxidoreductransaminasee, or 6-aminocaproate N-acetyltransferase, a [(6-acetamidohexanoyl)oxy]phosphonate (6-AAHOP) acyltransferase and 6-acetamidohexanoyl-CoA oxidoreductransaminasee, or a 3-oxo-6-aminopimeloyl-CoA oxidoreductransaminasee, a 3-hydroxy-6-aminopimeloyl-CoA dehydratransaminasee and a homolysine decarboxylase, or a 2-oxo-4-hydroxy-7-aminoheptanoate TAolase, a 2-oxo-7-aminohept-3-enoate reductransaminasee and a homolysine decarboxylase, or a 6-acetamidohexanoate reductransaminasee, a 6-acetamidohexanal aminotransferase and a 6-acetamidohexanamine N-acetyltransferase. In the case of 6ACA, 6-aminocaproate semialdehyde or HMD, by reference to FIG. 10, at least three exogenous nucleic acids can encode the enzymes such as the combination of enzymes represented by steps 10A, 10B and 10C; 10B, 10C and 10D; 10C, 10D and 10E; 10D, 10E and 10F; 10E, 10F and/or 10I; 10F, 10I and 10J, or any combination thereof of three, four, five, six, seven and/or eight of the enzymes represented by steps 10A, 10B, 10C, 10D, 10E, 10F, 10I and/or 10J. Similarly, any combination of four or more enzymes of a biosynthetic pathway as disclosed herein can be included in a non-naturally occurring microbial organism, as desired, so long as the combination of enzymes of the desired biosynthetic pathway results in production of the corresponding desired product.

In addition to the biosynthesis of 6-aminocaproic acid, caprolactam, hexamethylenediamine or 1,6-hexanediol as described herein, the non-naturally occurring microbial organisms and methods also can be utilized in various combinations with each other and with other microbial organisms and methods well known in the art to achieve product biosynthesis by other routes. For example, one alternative to produce 6-aminocaproic acid, caprolactam, hexamethylenediamine or 1,6-hexanediol other than use of the 6-aminocaproic acid, caprolactam, hexamethylenediamine or 1,6-hexanediol producers is through addition of another microbial organism capable of converting an adipate, 6-aminocaproic acid or caprolactam pathway intermediate to 6-aminocaproic acid, caprolactam, hexamethylenediamine or 1,6-hexanediol. One such procedure includes, for example, the fermentation of a microbial organism that produces a 6-aminocaproic acid, caprolactam, hexamethylenediamine or 1,6-hexanediol pathway intermediate. The 6-aminocaproic acid, caprolactam, hexamethylenediamine or 1,6-hexanediol pathway intermediate can then be used as a substrate for a second microbial organism that converts the 6-aminocaproic acid, caprolactam, hexamethylenediamine or 1,6-hexanediol pathway intermediate to 6-aminocaproic acid, caprolactamor hexamethylenediamine. The 6-aminocaproic acid, caprolactam, hexamethylenediamine or 1,6-hexanediol pathway intermediate can be added directly to another culture of the second organism or the original culture of 6-aminocaproic acid, caprolactam, hexamethylenediamine or 1,6-hexanediol pathway intermediate producers can be depleted of these microbial organisms by, for example, cell separation, and then subsequent addition of the second organism to the fermentation broth can be utilized to produce the final product without intermediate purification steps.

In other embodiments, the non-naturally occurring microbial organisms and methods can be assembled in a wide variety of sub pathways to achieve biosynthesis of, for example, 6-aminocaproic acid, caprolactam, hexamethylenediamine or 1,6-hexanediol. In these embodiments, biosynthetic pathways for a desired product can be segregated into different microbial organisms, and the different microbial organisms can be co-cultured to produce the final product. In such a biosynthetic scheme, the product of one microbial organism is the substrate for a second microbial organism until the final product is synthesized. For example, the biosynthesis of 6-aminocaproic acid, caprolactam, hexamethylenediamine or 1,6-hexanediol can be accomplished by constructing a microbial organism that contains biosynthetic pathways for conversion of one pathway intermediate to another pathway intermediate or the product. Alternatively, 6-aminocaproic acid, caprolactam, hexamethylenediamine or 1,6-hexanediol also can be biosynthetically produced from microbial organisms through co-culture or co-fermentation using two organisms in the same vessel, where the first microbial organism produces a 6-aminocaproic acid, caprolactam, hexamethylenediamine or 1,6-hexanediol intermediate and the second microbial organism converts the intermediate to 6-aminocaproic acid, caprolactam, hexamethylenediamine or 1,6-hexanediol.

Given the teachings and guidance provided herein, those skilled in the art will understand that a wide variety of combinations and permutations exist for the non-naturally occurring microbial organisms and methods together with other microbial organisms, with the co-culture of other non-naturally occurring microbial organisms having sub pathways and with combinations of other chemical and/or biochemical procedures well known in the art to produce 6-aminocaproic acid, caprolactam, hexamethylenediamine or 1,6-hexanediol.

Similarly, it is understood by those skilled in the art that a host organism can be selected based on desired characteristics for introduction of one or more gene disruptions to increase production of 6-aminocaproic acid, caprolactam, hexamethylenediamine or 1,6-hexanediol. Thus, it is understood that, if a genetic modification is to be introduced into a host organism to disrupt a gene, any homologs, orthologs or paralogs that catalyze similar, yet non-identical metabolic reactions can similarly be disrupted to ensure that a desired metabolic reaction is sufficiently disrupted. Because certain differences exist among metabolic networks between different organisms, those skilled in the art will understand that the actual genes disrupted in a given organism may differ between organisms. However, given the teachings and guidance provided herein, those skilled in the art also will understand that the methods can be applied to any suitable host microorganism to identify the cognate metabolic alterations needed to construct an organism in a species of interest that will increase 6-aminocaproic acid, caprolactam, hexamethylenediamine or 1,6-hexanediol biosynthesis. In a particular embodiment, the increased production couples biosynthesis of 6-aminocaproic acid, caprolactam, hexamethylenediamine or 1,6-hexanediol to growth of the organism, and can obligatorily couple production of 6-aminocaproic acid, caprolactam, hexamethylenediamine or 1,6-hexanediol to growth of the organism if desired and as disclosed herein.

Sources of encoding nucleic acids for a 6-aminocaproic acid, caprolactam, hexamethylenediamine or 1,6-hexanediol pathway enzyme can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. In some embodiments, the source of the encoding nucleic acids for a 6-aminocaproic acid, caprolactam, hexamethylenediamine or 1,6-hexanediol pathway enzyme is shown in Tables 6, 8 and 11. In some embodiments, the source of the encoding nucleic acids for transaminase enzyme is shown in Table 6. In some embodiments, the source of the encoding nucleic acids for transaminase enzyme is from the genus Achromobacter, Acidaminococcus, Collinsella, Peptostreptococcaceae, Paenarthrobacter or Romboustsia. In some embodiments, the source of the encoding nucleic acids for a 6-aminocaproic acid, caprolactam, hexamethylenediamine or 1,6-hexanediol pathway enzyme are species such as, Escherichia coli, Escherichia coli str. K12, Escherichia coli C, Escherichia coli W, Pseudomonas sp, Pseudomonas knackmussii, Pseudomonas sp. Strain B13, Pseudomonas putida, Pseudomonas fluorescens, Pseudomonas stutzeri, Pseudomonas mendocina, Rhodopseudomonas palustris, Mycobacterium tuberculosis, Vibrio cholera, Heliobacter pylori, Klebsiella pneumoniae, Serratia proteamaculans, Streptomyces sp. 2065, Pseudomonas aeruginosa, Pseudomonas aeruginosa PAO1, Ralstonia eutropha, Ralstonia eutropha H16, Clostridium acetobutylicum, Euglena gracilis, Treponema denticola, Clostridium kluyveri, Homo sapiens, Rattus norvegicus, Acinetobacter sp. ADP1, Acinetobacter sp. Strain M-1, Streptomyces coelicolor, Eubacterium barkeri, Peptostreptococcus asaccharolyticus, Clostridium botulinum, Clostridium botulinum A3 str, Clostridium tyrobutyricum, Clostridium pasteurianum, Clostridium thermoaceticum (Moorella thermoaceticum), Moorella thermoacetica Acinetobacter calcoaceticus, Mus musculus, Sus scrofa, Flavobacterium sp, Arthrobacter aurescens, Penicillium chrysogenum, Aspergillus niger, Aspergillus nidulans, Bacillus subtilis, Saccharomyces cerevisiae, Zymomonas mobilis, Mannheimia succiniciproducens, Clostridium ljungdahlii, Clostridium carboxydivorans, Geobacillus stearothermophilus, Agrobacterium tumefaciens, Achromobacter xylosoxidans, Achromobacter denitrificans, Arabidopsis thaliana, Haemophilus influenzae, Acidaminococcus fermentans, Clostridium sp. M62/1, Fusobacterium nucleatum, Bos taurus, Zoogloea ramigera, Rhodobacter sphaeroides, Clostridium beijerinckii, Metallosphaera sedula, Thermoanaerobacter species, Thermoanaerobacter brockii, Acinetobacter baylyi, Porphyromonas gingivalis, Leuconostoc mesenteroides, Sulfolobus tokodaii, Sulfolobus tokodaii 7, Sulfolobus solfataricus, Sulfolobus solfataricus, Sulfolobus acidocaldarius, Salmonella typhimurium, Salmonella enterica, Thermotoga maritima, Halobacterium salinarum, Bacillus cereus, Clostridium difficile, Alkaliphilus metalliredigenes, Thermoanaerobacter tengcongensis, Saccharomyces kluyveri, Helicobacter pylori, Corynebacterium glutamicum, Clostridium saccharoperbutylacetonicum, Pseudomonas chlororaphis, Streptomyces clavuligerus, Campylobacter jejuni, Thermus thermophilus, Pelotomaculum thermopropionicum, Bacteroides capillosus, Anaerotruncus colihominis, Natranaerobius thermophilius, Archaeoglobus fulgidus, Archaeoglobus fulgidus DSM 4304, Haloarcula marismortui, Pyrobaculum aerophilum, Pyrobaculum aerophilum str. IM2, Nicotiana tabacum, Menthe piperita, Pinus taeda, Hordeum vulgare, Zea mays, Rhodococcus opacus, Cupriavidus necator, Bradyrhizobium japonicum, Bradyrhizobium japonicum USDA110, Ascarius suum, butyrate-producing bacterium L2-50, Bacillus megaterium, Methanococcus maripaludis, Methanosarcina mazei, Methanosarcina mazei, Methanocarcina barkeri, Methanocaldococcus jannaschii, Caenorhabditis elegans, Leishmania major, Methylomicrobium alcaliphilum 20Z, Chromohalobacter salexigens, Archaeglubus fulgidus, Chlamydomonas reinhardtii, Trichomonas vaginalis G3, Trypanosoma brucei, Mycoplana ramose, Micrococcus luteas, Acetobacter pasteurians, Kluyveromyces lactis, Mesorhizobium loti, Lactococcus lactis, Lysinibacillus sphaericus, Candida boidinii, Candida albicans SC5314, Burkholderia ambifaria AMMD, Ascaris suun, Acinetobacter baumanii, Acinetobacter calcoaceticus, Burkholderia phymatum, Candida albicans, Clostridium subterminale, Cupriavidus taiwanensis, Flavobacterium lutescens, Lachancea kuyveri, Lactobacillus sp. 30a, Leptospira interrogans, Moorella thermoacetica, Myxococcus xanthus, Nicotiana glutinosa, Nocardia iowensis (sp. NRRL 5646), Pseudomonas reinekei MT1, Ralstonia eutropha JMP134, Ralstonia metallidurans, Rhodococcus jostii, Schizosaccharomyces pombe, Selenomonas ruminantium, Streptomyces clavuligenus, Syntrophus aciditrophicus, Vibrio parahaemolyticus, Vibrio vulrificus, as well as other exemplary species disclosed herein or available as source organisms for corresponding genes (see Examples). However, with the complete genome sequence available for now more than 550 species (with more than half of these available on public databases such as the NCBI), including 395 microorganism genomes and a variety of yeast, fungi, plant, and mammalian genomes, the identification of genes encoding the requisite 6-aminocaproic acid, caprolactam, hexamethylenediamine or 1,6-hexanediol biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations enabling biosynthesis of 6-aminocaproic acid, caprolactam, hexamethylenediamine or 1,6-hexanediol described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when a 6-aminocaproic acid, caprolactam, hexamethylenediamine or 1,6-hexanediol biosynthetic pathway exists in an unrelated species, 6-aminocaproic acid, caprolactam, hexamethylenediamine or 1,6-hexanediol biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods can be applied to all microbial organisms using the cognate metabolic alterations to those exemplified herein to construct a microbial organism in a species of interest that will synthesize 6-aminocaproic acid, caprolactam, hexamethylenediamine or 1,6-hexanediol.

Host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, and the like. For example, E. coli is a particularly useful host organism since it is a well characterized microbial organism suitable for genetic engineering. Other particularly useful host organisms include yeast such as Saccharomyces cerevisiae. It is understood that any suitable microbial host organism can be used to introduce metabolic and/or genetic modifications to produce a desired product.

Methods for constructing and testing the expression levels of a non-naturally occurring 6-aminocaproic acid, caprolactam, hexamethylenediamine or 1,6-hexanediol-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

Exogenous nucleic acid sequences involved in a pathway for production of 6-aminocaproic acid, caprolactam, hexamethylenediamine or 1,6-hexanediol can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more 6-aminocaproic acid, caprolactam, hexamethylenediamine or 1,6-hexanediol biosynthetic pathway encoding nucleic acids as exemplified herein operably linked to expression control sequences functional in the host organism. Expression vectors applicable for use in the microbial host organisms include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoTransaminase, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

In some embodiments are methods for producing a desired intermediate or product such as adipate, 6-aminocaproic acid, caprolactam, hexamethylenediamine or 1,6-hexanediol. For example, a method for producing adipate can involve culturing a non-naturally occurring microbial organism having an adipate pathway, the pathway including at least one exogenous nucleic acid encoding an adipate pathway enzyme expressed in a sufficient amount to produce adipate, under conditions and for a sufficient period of time to produce adipate, the adipate pathway including succinyl-CoA: acetyl-CoA acyl transferase, 3-hydroxyacyl-CoA dehydrogenase, 3-hydroxyadipyl-CoA dehydratransaminasee,adipate semialdehydereductransaminasee, and adipyl-CoA synthetransaminasee or phosphotransadipylase/adipate kinase or adipyl-CoA: acetyl-CoA transferase or adipyl-CoA hydrolase. Additionally, a method for producing adipate can involve culturing a non-naturally occurring microbial organism having an adipate pathway, the pathway including at least one exogenous nucleic acid encoding an adipate pathway enzyme expressed in a sufficient amount to produce adipate, under conditions and for a sufficient period of time to produce adipate, the adipate pathway including succinyl-CoA: acetyl-CoA acyl transferase, 3-oxoadipyl-CoA transferase, 3-oxoadipate reductransaminasee, 3-hydroxyadipate dehydratransaminasee, and 2-enoate reductransaminasee.

Further, a method for producing 6-aminocaproic acid can involve culturing a non-naturally occurring microbial organism having a 6-aminocaproic acid pathway, the pathway including at least one exogenous nucleic acid encoding a 6-aminocaproic acid pathway enzyme expressed in a sufficient amount to produce 6-aminocaproic acid, under conditions and for a sufficient period of time to produce 6-aminocaproic acid, the 6-aminocaproic acid pathway including CoA-dependent trans-enoyl-CoA reductransaminasee and transaminase or 6-aminocaproate dehydrogenase. Additionally, a method for producing caprolactam can involve culturing a non-naturally occurring microbial organism having a caprolactam pathway, the pathway including at least one exogenous nucleic acid encoding a caprolactam pathway enzyme expressed in a sufficient amount to produce caprolactam, under conditions and for a sufficient period of time to produce caprolactam, the caprolactam pathway including CoA-dependent aldehyde dehydrogenase, transaminase or 6-aminocaproate dehydrogenase, and amidohydrolase.

Suitable purification and/or assays to test for the production of 6-aminocaproic acid, caprolactam, hexamethylenediamine or 1,6-hexanediol can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme activities from the exogenous DNA sequences can also be assayed using methods well known in the art.

The 6-aminocaproic acid, caprolactam, hexamethylenediamine or 1,6-hexanediol can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Any of the non-naturally occurring microbial organisms described herein can be cultured to produce and/or secrete the biosynthetic products. For example, the 6-aminocaproic acid, caprolactam, hexamethylenediamine or 1,6-hexanediol producers can be cultured for the biosynthetic production of 6-aminocaproic acid, caprolactam, hexamethylenediamine or 1,6-hexanediol.

For the production of 6-aminocaproic acid, caprolactam, hexamethylenediamine or 1,6-hexanediol, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in U.S. Pat. No. 7,947,483 issued May 24, 2011. Fermentations can be performed in a batch, fed-batch or continuous manner, as disclosed herein.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium can include, for example, any carbohydrate source which can supply a source of carbon to the non-naturally occurring microorganism. Such sources include, for example, sugars such as glucose, xylose, arabinose, galactose, mannose, fructose, sucrose and starch. Other sources of carbohydrate include, for example, renewable feedstocks and biomass. Exemplary types of biomasses that can be used as feedstocks in the methods include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms for the production of 6-aminocaproic acid, caprolactam, hexamethylenediamine or 1,6-hexanediol.

In addition to renewable feedstocks such as those exemplified above, the 6-aminocaproic acid, caprolactam, hexamethylenediamine, or levulinic acid microbial organisms also can be modified for growth on syngas as its source of carbon. In this specific embodiment, one or more proteins or enzymes are expressed in the 6-aminocaproic acid, caprolactam, hexamethylenediamine or 1,6-hexanediol producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H2 and CO and can be obtained from the gasification of any organic feedstock, including but not limited to coal, coal oil, natural gas, biomass, and waste organic matter. Gasification is generally carried out under a high fuel to oxygen ratio. Although largely H2 and CO, syngas can also include CO2 and other gases in smaller quantities. Thus, synthesis gas provides a cost effective source of gaseous carbon such as CO and additionally, CO2.

The Wood-Ljungdahl pathway catalyzes the conversion of CO and H2 to acetyl-CoA and other products such as acetate. Organisms capable of utilizing CO and syngas also generally have the capability of utilizing CO2 and CO2/H2 mixtures through the same basic set of enzymes and transformations encompassed by the Wood-Ljungdahl pathway. H2-dependent conversion of CO2 to acetate by microorganisms was recognized long before it was revealed that CO also could be used by the same organisms and that the same pathways were involved. Many acetogens have been shown to grow in the presence of CO2 and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

2CO2+4H2+n ADP+n Pi→CH₃COOH+2H2O+n ATP

Hence, non-naturally occurring microorganisms possessing the Wood-Ljungdahl pathway can utilize CO2 and H2 mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-THF to acetyl-CoA. The reactions in the methyl branch are catalyzed in order by the following enzymes: ferredoxin oxidoreductransaminasee, formate dehydrogenase, formyltetrahydrofolate synthetransaminasee, methenyltetrahydrofolate cyclodehydratransaminasee, methylenetetrahydrofolate dehydrogenase and methylenetetrahydrofolate reductransaminasee. The reactions in the carbonyl branch are catalyzed in order by the following enzymes or proteins: cobalamide corrinoid/iron-sulfur protein, methyltransferase, carbon monoxide dehydrogenase, acetyl-CoA synthase, acetyl-CoA synthase disulfide reductransaminasee and hydrogenase, and these enzymes can also be referred to as methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC). Following the teachings and guidance provided herein for introducing a sufficient number of encoding nucleic acids to generate a 6-aminocaproic acid, caprolactam, hexamethylenediamine or 1,6-hexanediol pathway, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms such that the modified organism contains the complete Wood-Ljungdahl pathway will confer syngas utilization ability.

Additionally, the reductive (reverse) tricarboxylic acid cycle coupled with carbon monoxide dehydrogenase and/or hydrogenase activities can also be used for the conversion of CO, CO2 and/or H2 to acetyl-CoA and other products such as acetate. Organisms capable of fixing carbon via the reductive TCA pathway can utilize one or more of the following enzymes: ATP citrate-lyase, citrate lyase, aconitransaminase, isocitrate dehydrogenase, alpha-ketoglutarate: ferredoxin oxidoreductransaminasee, succinyl-CoA synthetransaminasee, succinyl-CoA transferase, fumarate reductransaminasee, fumarase, malate dehydrogenase, NAD(P)Ferredoxin oxidoreductransaminasee, carbon monoxide dehydrogenase, and hydrogenase. Specifically, the reducing equivalents extracted from CO and/or H2 by carbon monoxide dehydrogenase and hydrogenase are utilized to fix CO2 via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetransaminasee. Acetyl-CoA can be converted to the p-toluate, terepathalate, or (2-hydroxy-3-methyl-4-oxobutoxy) phosphonate precursors, glyceraldehyde-3-phosphate, phosphoenolpyruvate, and pyruvate, by pyruvate: ferredoxin oxidoreductransaminasee and the enzymes of gluconeogenesis. Following the teachings and guidance provided herein for introducing a sufficient number of encoding nucleic acids to generate a p-toluate, terephthalate or (2-hydroxy-3-methyl-4-oxobutoxy) phosphonate pathway, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the microbial organisms such that the modified organism contains the complete reductive TCA pathway will confer syngas utilization ability.

Given the teachings and guidance provided herein, those skilled in the art will understand that a non-naturally occurring microbial organism can be produced that secretes the biosynthesized compounds when grown on a carbon source such as a carbohydrate. Such compounds include, for example, 6-aminocaproic acid, caprolactam, hexamethylenediamine or 1,6-hexanediol and any of the intermediate metabolites in the 6-aminocaproic acid, caprolactam, hexamethylenediamine or 1,6-hexanediol pathway. All that is required is to engineer in one or more of the required enzyme activities to achieve biosynthesis of the desired compound or intermediate including, for example, inclusion of some or all of the 6-aminocaproic acid, caprolactam, hexamethylenediamine or 1,6-hexanediol biosynthetic pathways. Accordingly, some embodiments provide a non-naturally occurring microbial organism that produces and/or secretes 6-aminocaproic acid, caprolactam, hexamethylenediamine or 1,6-hexanediol when grown on a carbohydrate and produces and/or secretes any of the intermediate metabolites shown in the 6-aminocaproic acid, caprolactam, hexamethylenediamine or 1,6-hexanediol pathway when grown on a carbohydrate. For example, an adipate producing microbial organisms can initiate synthesis from an intermediate, for example, 3-oxoadipyl-CoA, 3-hydroxyadipyl-CoA, 5-carboxy-2-pentenoyl-CoA, or adipyl-CoA (see FIG. 1), as desired. In addition, an adipate producing microbial organism can initiate synthesis from an intermediate, for example, 3-oxoadipyl-CoA, 3-oxoadipate, 3-hydroxyadipate, or hexa-2-enedioate. The 6-aminocaproic acid producing microbial organism can initiate synthesis from an intermediate, for example, adipate semialdehyde. The caprolactam producing microbial organism can initiate synthesis from an intermediate, for example, adipate semialdehyde or 6-aminocaproic acid (see FIG. 1), as desired.

TABLE 4
Activity of Aldehyde Dehydrogenases on Adipyl-CoA

| | | Accession | Activity - | Activity - |
|---|---|---|---|---|
| NO. | Organism | No/SEQ ID NO. | NADH | NADPH |
| 1 | Clostridium kluyveri DSM555 | SEQ ID NO: 141 | − | + |
| 2 | Porphyromonas gingivalis W83 | SEQ ID NO: 142 | + | − |
| 3 | Clostridium difficile 630 | SEQ ID NO: 143 | − | + |
| 4 | Kluyvera intestini | WP_071196317.1 | + | − |
| 5 | Clostridium neonatale | WP_058295546.1 | − | − |
| 6 | Aerococcus sp. HMSC062B07 | WP_070558456.1 | − | − |
| 7 | Peptostreptococcaceae bacterium | WP_021676458.1 | + | − |
| | oral | | | |
| 8 | Dasania marina | WP_026244399.1 | − | − |
| 9 | Porphyromonadaceae bacterium | WP_036830068.1 | − | − |
| | COT-184 | | | |
| 10 | Clostridium lundense | WP_027623222.1 | − | − |
| 11 | Anaerocolumna jejuensis | WP_073279774.1 | + | − |
| 12 | Clostridium homopropionicum | WP_052222510.1 | − | − |
| 13 | Geosporobacter ferrireducens | WP_069981616.1 | − | − |
| 14 | Listeria ivanovii | WP_038407128.1 | − | − |
| 15 | Bacillus soli | WP_066062455.1 | + | − |
| 16 | Enterococcus rivorum | WP_069697141.1 | − | − |
| 17 | Desnuesiella massiliensis | WP_055665162.1 | + | − |
| 18 | Bacteroidales bacterium KA00251 | WP_066041885.1 | − | − |
| 19 | Caldanaerobius | WP_026487268.1 | − | − |
| | polysaccharolyticus | | | |
| 20 | Clostridium sp. ASF356 | WP_004036483.1 | + | − |
| 21 | Clostridiales bacterium DRI-13 | WP_034420506.1 | − | − |
| 22 | Fusobacterium ulcerans ATCC | WP_005981617.1 | − | − |
| | 49185 | | | |
| 23 | Anaerocolumna jejuensis | WP_073279351.1 | − | − |
| 24 | Cellulosilyticum sp. I15G10I2 | WP_070001026.1 | + | − |
| 25 | Geosporobacter ferrireducens | WP_083273866.1 | + | − |
| 26 | Pelosinus sp. UFO1 | WP_038668911.1 | − | − |
| 27 | Bacillus korlensis | WP_084362095.1 | + | − |
| 28 | Acidaminococcus massiliensis | WP_075579339.1 | + | − |
| 29 | Eubacterium sp. SB2 | WP_050640767.1 | − | − |
| 30 | Erwinia teleogrylli | WP_058911295.1 | − | − |
| 31 | Lachnospiraceae bacterium 32 | WP_016223553.1 | + | − |
| 32 | Eubacterium plexicaudatum | WP_004061597.1 | + | − |
| 33 | Clostridium sp. KNHs205 | WP_033166114.1 | + | − |
| 34 | Butyricimonas virosa | WP_027200274.1 | − | − |
| 35 | Malonomonas rubra | WP_072908980.1 | − | − |
| 36 | Robinsoniella peoriensis | WP_044292972.1 | + | − |
| 37 | Clostridium taeniosporum | WP_069679818.1 | − | − |
| 38 | Caldithrix abyssi | WP_006928331.1 | + | − |
| 39 | Piscicoccus intestinalis | WP_084343789.1 | − | − |
| 40 | Sporomusa sphaeroides | WP_075753933.1 | + | − |
| 41 | Bacillus sp. FJAT-25547 | WP_057762439.1 | + | − |
| 42 | Dorea sp. D27 | WP_049729435.1 | + | − |
| 43 | Oscillibacter sp. 13 | WP_081646270.1 | − | − |
| 44 | Enterococcus phoeniculicola | WP_010767571.1 | + | − |
| 45 | Blautia schinkii | WP_044941637.1 | + | − |
| 46 | Shuttleworthia satelles DSM 14600 | WP_006905683.1 | − | − |
| 47 | Clostridium intestinale | WP_073018444.1 | + | − |
| 48 | Massilioclostridium coli | WP_069989048.1 | − | − |
| 49 | Cloacibacillus porcorum | WP_066745012.1 | − | − |
| 50 | Clostridium sp. CL-2 | WP_032120205.1 | − | − |
| 51 | Clostridia bacterium UC5.1-1D10 | WP_054330586.1 | − | − |
| 52 | Methylobacterium sp. CCH5-D2 | WP_082772960.1 | − | − |
| 53 | Sporosarcina globispora | WP_053435653.1 | + | + |
| 54 | Lachnospiraceae bacterium | WP_031546337.1 | − | − |
| | AC3007 | | | |
| 55 | Lachnospiraceae bacterium 28-4 | WP_016290199.1 | − | − |
| 56 | Enterococcus avium | WP_034875865.1 | − | − |
| 57 | Desulfotomaculum | WP_027356260.1 | − | − |
| | thermocisternum | | | |
| 58 | Rhodobacter aestuarii | WP_076486054.1 | + | − |
| 59 | Clostridium grantii | WP_073337420.1 | + | − |
| 60 | Collinsella sp. GD7 | WP_066830323.1 | + | − |
| 61 | Clostridium estertheticum | WP_071611886.1 | − | − |
| 62 | bacterium MS4 | WP_038325413.1 | − | − |
| 63 | Clostridium glycyrrhizinilyticum | WP_009268007.1 | + | − |
| 64 | Bacillus horikoshii | WP_082892049.1 | − | − |
| 65 | Thermincola ferriacetica | WP_052218568.1 | + | − |
| 66 | Lachnospiraceae bacterium | WP_035653923.1 | + | − |
| | AC3007 | | | |
| 67 | Eubacterium sp. 14-2 | WP_016216571.1 | + | − |
| 68 | Candidatus Marispirochaeta | WP_069895590.1 | − | − |
| | associata | | | |
| 69 | Clostridium drakei | WP_032078293.1 | − | − |
| 70 | Halanaerobium kushneri | WP_076543773.1 | − | − |
| 71 | Clostridium fallax | WP_072896506.1 | − | − |
| 72 | Flavonifractor plautii | WP_009261118.1 | − | − |
| 73 | Clostridium propionicum | WP_066049640.1 | − | − |
| 74 | Anaerosalibacter massiliensis | WP_042682918.1 | + | − |
| 75 | Clostridium indolis DSM 755 | WP_024295710.1 | + | − |
| 76 | Gabonibacter massiliensis | WP_059027034.1 | − | − |
| 77 | Catabacter hongkongensis | WP_046444791.1 | + | + |
| 78 | Desulfitibacter alkalitolerans | WP_028307735.1 | − | − |
| 79 | Porphyromonas levii | WP_018357742.1 | − | − |
| 80 | Bacillus thermotolerans | WP_039235348.1 | + | − |
| 81 | Desulfitibacter alkalitolerans | WP_028307055.1 | − | − |
| 82 | Gracilibacillus kekensis | WP_073203236.1 | + | + |
| 83 | Lactonifactor longoviformis | WP_072848455.1 | − | − |
| 84 | Propionispora sp. 2/2-37 | WP_054258533.1 | + | − |
| 85 | Erysipelothrix larvae | WP_067632640.1 | − | − |
| 86 | Clostridium chauvoei | WP_021875658.1 | + | − |
| 87 | Thermoanaerobacterium | WP_014757178.1 | + | − |
| | aotearoense | | | |
| 88 | Ruminococcus sp. AT10 | WP_059066688.1 | + | − |
| 89 | Porphyromonas sp. HMSC077F02 | WP_070707924.1 | − | − |
| 90 | Acetobacterium dehalogenans | WP_026396046.1 | + | − |
| 91 | Spirochaeta alkalica | WP_018526526.1 | + | − |
| 92 | Alistipes sp. ZOR0009 | WP_047449305.1 | − | − |
| 93 | Clostridiisalibacter paucivorans | WP_026895448.1 | − | − |
| 94 | Clostridium caminithermale DSM | WP_073149471.1 | + | + |
| | 15212 | | | |
| 95 | Caldanaerobius fijiensis | WP_073341480.1 | + | − |
| 96 | Clostridium kluyveri | WP_073539833.1 | − | − |
| 97 | Pelosinus fermentans | WP_007958399.1 | + | − |
| 98 | Halanaerobium saccharolyticum | WP_005487288.1 | − | − |
| | subsp. saccharolyticum DSM 6643 | | | |
| 99 | Anaeroarcus burkinensis DSM | WP_018702299.1 | − | − |
| | 6283 | | | |
| 100 | Blautia wexlerae | WP_026648408.1 | + | − |
| 101 | Paenibacillus sp. OSY-SE | WP_019424162.1 | + | − |
| 102 | Brachyspira intermedia PWSA | WP_014488056.1 | − | − |
| 103 | Spirochaetes bacterium | OHD32879.1 | + | − |
| | GWC2_52_13 | | | |
| 104 | Thermoanaerobacterales bacterium | KUK31085.1 | − | − |
| | 50_218 | | | |
| 105 | Cohaesibacter marisflavi | WP_090072157.1 | − | − |
| 106 | Gracilibacillus ureilyticus | WP_089739945.1 | − | − |
| 107 | Romboutsia lituseburensis DSM | WP_092724914.1 | + | − |
| 108 | uncultured Clostridium sp. | SCJ29526.1 | − | − |
| 109 | Clostridium sp. CAG: 448 | CDC62685.1 | + | − |
| 110 | Clostridium ultunense Esp | CCQ95129.1 | − | − |
| 111 | Yersinia bercovieri ATCC 43970 | WP_005274635.1 | + | − |
| 112 | Proteocatella sphenisci | WP_028829945.1 | + | − |
| 113 | Clostridium sp. MSTE9 | WP_009063988.1 | − | − |
| 114 | Spirochaeta africana | WP_014454236.1 | − | − |
| 115 | Deltaproteobacteria bacterium | OGQ13386.1 | − | − |
| | RIFCSPHIGHO2_02_FULL_40_11 | | | |
| 116 | Clostridiales bacterium | KKM11466.1 | − | − |
| | PH28_bin88 | | | |
| 117 | Pelosinus propionicus DSM | WP_090932308.1 | + | − |
| 118 | Propionispora vibrioides | WP_091747803.1 | − | − |
| 119 | Natronincola ferrireducens | WP_090549432.1 | − | − |
| 120 | uncultured Ruminococcus sp. | WP_112331601.1 | − | − |
| 121 | Firmicutes bacterium CAG: 41 | WP_022229858.1 | − | − |
| 122 | Tannerella sp. oral | ETK11816.1 | − | − |
| 123 | Clostridium sp. DL-VIII | WP_009171375.1 | − | − |
| 124 | Desulfobulbus japonicus | WP_028581706.1 | − | − |
| 125 | Veillonella sp. oral | WP_009353657.1 | − | − |
| 126 | Bacillus selenitireducens | WP_013174003.1 | − | − |
| 127 | Deltaproteobacteria bacterium | OGP02283.1 | − | − |
| | GWA2_38_16 | | | |
| 128 | Clostridiaceae bacterium BRH | KJS20094.1 | − | − |
| 129 | Clostridium cadaveris | WP_035770223.1 | − | − |
| 130 | Vibrio hangzhouensis | WP_103880502.1 | − | − |
| 131 | Halanaerobium congolense | SDI24694.1 | − | − |
| 132 | uncultured Eubacterium sp. | SCH28733.1 | − | − |
| 133 | Oscillibacter sp. CAG: 241 | CDB26907.1 | − | − |
| 134 | Clostridium sp. KLE | ERI68946.1 | + | − |
| 135 | Caldalkalibacillus thermarum | WP_007505383.1 | + | − |
| | TA2.A1 | | | |
| 136 | Budvicia aquatica | WP_029095874.1 | − | − |
| 137 | Caldalkalibacillus thermarum | WP_007505383.1 | + | − |
| | TA2.A1 | | | |
| 138 | Rhodospirillum rubrum ATCC | WP_011388669.1 | − | − |
| | 11170 | | | |
| 139 | Bacteroidetes bacterium | OFX78235.1 | − | − |
| | GWE2_39_28 | | | |
| 140 | Desulfosporosinus sp. BICA1 | KJS46946.1 | − | − |
| 141 | Clostridium uliginosum | WP_090094411.1 | − | − |
| 142 | Pseudobutyrivibrio sp. ACV-2 | WP_090301343.1 | − | − |
| 143 | Sporolituus thermophilus DSM | WP_093690468.1 | − | − |
| 144 | Eubacteriaceae bacterium | WP_087275421.1 | − | − |
| | CHKCI004 | | | |
| 145 | Blautia sp. CAG: 257 | CDA04862.1 | + | − |
| 146 | Listeria marthii FSL | EFR88049.1 | + | − |
| 147 | Desulfosporosinus sp. OT | WP_009624792.1 | − | − |
| 148 | Clostridium methoxybenzovorans | WP_024346771.1 | + | − |
| 149 | Bacillus sp. m3-13 | WP_010197697.1 | + | − |
| 150 | bacterium CG2_30_54_10 | OIP28307.1 | + | − |
| 151 | Halanaerobium sp. 4-GBenrich | ODS50009.1 | − | − |
| 152 | Candidatus Izimaplasma sp. | KFZ26741.1 | + | + |
| 153 | Desulfotomaculum guttoideum | WP_092244224.1 | − | − |
| 154 | Bacillus daliensis | WP_090843272.1 | − | − |
| 155 | Sporomusa acidovorans | WP_093796665.1 | − | − |
| 156 | Clostridium sp. C105KSO15 | WP_089994985.1 | − | − |
| 157 | Firmicutes bacterium CAG: 41 | CCZ36420.1 | + | − |
| 158 | Fusobacterium nucleatum subsp. | WP_085057258.1 | + | − |
| 159 | Thermoanaerobacterium | WP_013788835.1 | + | − |
| | xylanolyticum LX-11 | | | |
| 160 | Enterococcus pallens | WP_010758150.1 | − | − |
| 161 | Porphyromonas uenonis | WP_007364879.1 | − | − |
| 162 | Tenericutes bacterium | OHE32257.1 | − | − |
| | GWD2_38_27 | | | |
| 163 | Clostridia bacterium BRH_c25 | KU067763.1 | − | − |
| 164 | Listeria monocytogenes | WP_012951491.1 | + | − |
| 165 | Clostridium lavalense | WP_092361844.1 | + | − |
| 166 | Acetanaerobacterium elongatum | WP_092640331.1 | + | − |
| 167 | Alkaliphilus peptidifermentans | WP_091539210.1 | + | − |
| | DSM | | | |
| 168 | Clostridium sp. C105KSO15 | WP_089983798.1 | − | − |
| 169 | Ruminococcus sp. CAG: 17 | CCY97458.1 | − | − |
| 170 | Clostridium hylemonae DSM 15053 | EEG72288.1 | − | − |
| 171 | Acetonema longum DSM 6540 | EGO64744.1 | − | − |
| 172 | Brachyspira innocens | WP_020003501.1 | − | − |
| 173 | Clostridium saccharobutylicum | WP_022747467.1 | − | − |
| 174 | Tenericutes bacterium | OHE28831.1 | − | − |
| | GWD2_38_27 | | | |
| 175 | Bacillus sp. FJAT-25547 | WP_053476394.1 | − | − |
| 176 | Clostridium populeti | WP_092561044.1 | + | − |
| 177 | Natronincola peptidivorans | WP_090442614.1 | − | − |
| 178 | Megasphaera paucivorans | WP_091652222.1 | − | − |
| 179 | Anaerobium acetethylicum | WP_091232027.1 | − | − |
| 180 | Eubacterium limosum | ALU13318.1 | − | − |
| 181 | Porphyromonas sp. CAG: 1061 | CCY08492.1 | − | − |
| 182 | Clostridium beijerinckii strain | AAD31841.1 | − | − |
| | NRRL B593 | | | |
| 183 | Clostridium sticklandii DSM 519 | WP_013360893.1 | − | − |
| 184 | Bacillus oryziterrae | WP_017754440.1 | − | − |
| 185 | Yersinia enterocolitica | WP_005157703.1 | − | − |
| 186 | Syntrophobacterales bacterium | OHE18777.1 | − | − |
| | GWC2_56_13 | | | |
| 187 | Candidatus Bacteroides | KQM08700.1 | + | − |
| | periocalifornicus | | | |
| 188 | Anaerocolumna aminovalerica | WP_091689178.1 | + | − |
| 189 | Natronincola peptidivorans | WP_090439673.1 | − | − |
| 190 | Dendrosporobacter quercicolus | WP_092070189.1 | − | − |
| 191 | uncultured Flavonifractor sp. | SCJ32847.1 | − | − |
| 192 | Geobacillussp. Y4.1MC1 | OUM85091.1 | − | − |
| 193 | Clostridium bolteae CAG: 59 | CCX97030.1 | − | − |
| 194 | Roseburia inulinivorans A2-194 | WP_118109132.1 | − | − |

TABLE 5
5-carboxy-2-pentenoyl-CoA reductases

| Candidate | | Accession No/ | Activity | Activity |
|---|---|---|---|---|
| # | Species | SEQ ID NO. | CP-CoA | Cr-CoA |
| 1 | Candida | XP_026596596.1/ | + | + |
| | tropicalis | SEQ ID NO: 144 | | |
| 2 | Coraliomargarita | WP_013043307.1 | − | ND |
| | akajimensis | | | |
| | DSM 45221 | | | |
| 3 | Vibrio | WP_009599222.1 | − | ND |
| | caribbenthicus | | | |
| | ATCC BAA- | | | |
| | 2122 | | | |
| 4 | Cephaloticoccus | WP_068629633.1 | + | ND |
| | primus | | | |
| 5 | Opitutaceae | WP_068772922.1 | + | ND |
| | TSB47 | | | |
| 6 | Puniceicoccaceae | PDH30576.1 | + | ND |
| | MEDG31 | | | |
| 7 | Rubritalea | WP_018969548.1 | + | ND |
| | marina | | | |
| 8 | Rubritalea | WP_105041881.1 | + | ND |
| | profundi | | | |
| 9 | Rubritalea | SHK09753.1 | + | ND |
| | squalenifa | | | |
| 10 | Verrucomicrobiae | PAW64593.1 | − | ND |
| | bacterium | | | |
| 11 | Vibrio | WP_053410267.1 | + | ND |
| | hepatarius | | | |
| 12 | Vibrio | WP_065302224.1 | − | ND |
| | natriegens | | | |
| 13 | Vibrio owensii | WP_009705827.1 | − | ND |
| | LMG 25430 | | | |
| 14 | Vibrio sp. EJY3 | WP_014231565.1 | + | ND |
| 15 | Arabidopsis | CAB75790.1 | + | ND |
| | thaliana | | | |
| 16 | Rattus | NP_058905.1 | − | ND |
| | norvegicus | | | |
| 17 | Coraliomargarita | OUU72757.1 | + | ND |
| | sp. | | | |
| 18 | Haloferula sp. | WP_035604676.1 | + | ND |
| 19 | Opitutaceae | KRP37089.1 | + | ND |
| | BACL24 | | | |
| 20 | Opitutaceae | WP_107744621.1 | + | ND |
| | EW11 | | | |
| 21 | Puniceicoccaceae | PDH31188.1 | + | ND |
| | MED-G30 | | | |
| 22 | Terrimicrobium | WP_075078790.1 | − | ND |
| | sacchariphilum | | | |
| 23 | Vibrio owensii | WP_038894812.1 | + | ND |
| | ATCC | | | |
| 24 | Drosophila | NP_610914.2 | + | + |
| | melanogaster | SEQ ID NO: 145 | | |
| 25 | Homo sapiens | NP_057095.4 | + | ND |
| 26 | Euglena gracilis | Truncated version | + | + |
| | | of Q5EU90.1 | | |

The non-naturally occurring microbial organisms are constructed using methods well known in the art as exemplified herein to exogenously express at least one nucleic acid encoding a 6-aminocaproic acid, caprolactam, hexamethylenediamine or 1,6-hexanediol pathway enzyme in sufficient amounts to produce 6-aminocaproic acid, caprolactam, hexamethylenediamine or 1,6-hexanediol. It is understood that the microbial organisms are cultured under conditions sufficient to produce 6-aminocaproic acid, caprolactam, hexamethylenediamine or 1,6-hexanediol. Following the teachings and guidance provided herein, the non-naturally occurring microbial organisms can achieve biosynthesis of 6-aminocaproic acid, caprolactam, hexamethylenediamine or 1,6-hexanediol resulting in intracellular concentrations between about 0.1-200 mM or more. Generally, the intracellular concentration of 6-aminocaproic acid, caprolactam, hexamethylenediamine or 1,6-hexanediol is between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the non-naturally occurring microbial organisms.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. Pat. No. 7,947,483, issued May 24, 2011. Any of these conditions can be employed with the non-naturally occurring microbial organisms as well as other anaerobic conditions well known in the art. Under such anaerobic conditions, the 6-aminocaproic acid, caprolactam, hexamethylenediamine or 1,6-hexanediol producers can synthesize 6-aminocaproic acid, caprolactam, hexamethylenediamine or 1,6-hexanediol at intracellular concentrations of 5-10 mM or more as well as all other concentrations exemplified herein. It is understood that, even though the above description refers to intracellular concentrations, 6-aminocaproic acid, caprolactam, hexamethylenediamine or 1,6-hexanediol producing microbial organisms can produce 6-aminocaproic acid, caprolactam, hexamethylenediamine or 1,6-hexanediol intracellularly and/or secrete the product into the culture medium.

The culture conditions can include, for example, liquid culture procedures as well as fermentation and other large scale culture procedures. As described herein, particularly useful yields of the biosynthetic products can be obtained under anaerobic or substantially anaerobic culture conditions.

As described herein, one exemplary growth condition for achieving biosynthesis of 6-aminocaproic acid, caprolactam, hexamethylenediamine or 1,6-hexanediol includes anaerobic culture or fermentation conditions. In certain embodiments, the non-naturally occurring microbial organisms can be sustained, cultured or fermented under anaerobic or substantially anaerobic conditions. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also include growing or resting cells in liquid medium or on solid agar inside a sealed chamber maintained with an atmosphere of less than 1% oxygen. The percent of oxygen can be maintained by, for example, sparging the culture with an N2/CO2 mixture or other suitable non-oxygen gas or gases.

The culture conditions described herein can be scaled up and grown continuously for manufacturing of 6-aminocaproic acid, caprolactam, hexamethylenediamine or 1,6-hexanediol. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of 6-aminocaproic acid, caprolactam, hexamethylenediamine or 1,6-hexanediol. Generally, and as with non-continuous culture procedures, the continuous and/or near-continuous production of 6-aminocaproic acid, caprolactam, hexamethylenediamine or 1,6-hexanediol will include culturing a non-naturally occurring 6-aminocaproic acid, caprolactam, hexamethylenediamine or 1,6-hexanediol producing organism in sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, organisms can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of 6-aminocaproic acid, caprolactam, hexamethylenediamine or 1,6-hexanediol can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the 6-aminocaproic acid, caprolactam, hexamethylenediamine or 1,6-hexanediol producers for continuous production of substantial quantities of 6-aminocaproic acid, caprolactam, hexamethylenediamine or 1,6-hexanediol, the 6-aminocaproic acid, caprolactam, hexamethylenediamine or 1,6-hexanediol producers also can be, for example, simultaneously subjected to chemical synthesis procedures to convert the product to other compounds or the product can be separated from the fermentation culture and sequentially subjected to chemical conversion to convert the product to other compounds, if desired. As described herein, an intermediate in the adipate pathway utilizing 3-oxoadipate, hexa-2-enedioate, can be converted to adipate, for example, by chemical hydrogenation over a platinum catalyst.

As described herein, exemplary growth conditions for achieving biosynthesis of 6-aminocaproic acid, caprolactam, hexamethylenediamine or 1,6-hexanediol includes the addition of an osmoprotectant to the culturing conditions. In certain embodiments, the non-naturally occurring microbial organisms can be sustained, cultured or fermented as described above in the presence of an osmoprotectant. Briefly, an osmoprotectant means a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethylslfonioproprionate, 3-dimethylsulfonio-2-methylproprionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. For example, Escherichia coli in the presence of varying amounts of 6-aminocaproic acid is suitably grown in the presence of 2 mM glycine betaine. The amount of osmoprotectant in the culturing conditions can be, for example, no more than about 0. 1 mM, no more than about 0. 5 mM, no more than about 1. 0 mM, no more than about 1. 5 mM, no more than about 2. 0 mM, no more than about 2. 5 mM, no more than about 3. o mM, no more than about 5. 0 mM, no more than about 7. 0 mM, no more than about 10 mM, no more than about 50 mM, no more than about 100 mM or no more than about 500 mM.

Successfully engineering a pathway involves identifying an appropriate set of enzymes with sufficient activity and specificity. This entails identifying an appropriate set of enzymes, cloning their corresponding genes into a production host, optimizing fermentation conditions, and assaying for product formation following fermentation. To engineer a production host for the production of 6-aminocaproic acid or caprolactam, one or more exogenous DNA sequence(s) can be expressed in a host microorganism. In addition, the microorganism can have endogenous gene(s) functionally deleted. These modifications will allow the production of 6-aminocaproate or caprolactam using renewable feedstock.

In some embodiments minimizing or even eliminating the formation of the cyclic imine or caprolactam during the conversion of 6-aminocaproic acid to MD entails adding a functional group (for example, acetyl, succinyl) to the amine group of 6-aminocaproic acid to protect it from cyclization. This is analogous to ornithine formation from L-glutamate in Escherichia coli. Specifically, glutamate is first converted to N-acetyl-L-glutamate by N-acetylglutamate synthase. N-Acetyl-L-glutamate is then activated to N-acetylglutamyl-phosphate, which is reduced and transaminated to form N-acetyl-L-ornithine. The acetyl group is then removed from N-acetyl-L-ornithine by N-acetyl-L-ornithine deacetylase forming L-ornithine. Such a route is necessary because formation of glutamate-5-phosphate from glutamate followed by reduction to glutamate-5-semialdehyde leads to the formation of (S)-1-pyrroline-5-carboxylate, a cyclic imine formed spontaneously from glutamate-5-semialdehyde. In the case of forming HMD from 6-aminocaproic acid, the steps can involve acetylating 6-aminocaproic acid to acetyl-6-aminocaproic acid, activating the carboxylic acid group with a CoA or phosphate group, reducing, aminating, and deacetylating.

The invention additionally provides culture medium comprising bioderived HMD, 6-aminocaproate semialdehyde, and/or HDO, or other products disclosed herein, wherein the bioderived product has a carbon-12, carbon-13 and carbon-14 isotope ratio that reflects an atmospheric carbon dioxide uptake source. In a particular embodiment, the culture medium can be separated from a non-naturally occurring microbial organism having a HMD, 6-aminocaproate semialdehyde, and/or HDO pathway. In another embodiment, the invention provides bioderived a HMD, 6-aminocaproate semialdehyde, and/or HDO having a carbon-12, carbon-13 and carbon-14 isotope ratio that reflects an atmospheric carbon dioxide uptake source. In a particular embodiment, the bioderived HMD, 6-aminocaproate semialdehyde, and/or HDO of claims 61-62, can have an Fm value of at least 80%, at least 85%, at least 90%, at least 95% or at least 98%. Such bioderived products of the invention can be produced by the methods of the invention, as disclosed herein.

The invention further provides a composition comprising bioderived HMD, 6-aminocaproate semialdehyde, and/or HDO, and a compound other than the bioderived HMD, 6-aminocaproate semialdehyde, and/or HDO. The compound other than the bioderived product can be a trace amount of a cellular portion of a non-naturally occurring microbial organism of the invention having a HMD, 6-aminocaproate semialdehyde, and/or HDO. The composition can comprise, for example, bioderived HMD, 6-aminocaproate semialdehyde, and/or HDO, or a cell lysate or culture supernatant of a microbial organism of the invention. In some embodiments, the invention provides a composition comprising bioderived a HMD, 6-aminocaproate semialdehyde, and/or HDO having a carbon-12, carbon-13 and carbon-14 isotope ratio that reflects an atmospheric carbon dioxide uptake source. In a particular embodiment, the bioderived HMD, 6-aminocaproate semialdehyde, and/or HDO of claims 61-62, can have an Fm value of at least 80%, at least 85%, at least 90%, at least 95% or at least 98%. Compositions comprising such bioderived products of the invention can be produced by the methods of the invention, as disclosed herein.

### EXAMPLES

### Example 1. Identification of Transaminases with Activity on 6-Aminocaproic Acid (6ACA)

Genes encoding transaminases were identified bioinformatically from metagenomic libraries and public databases using a basic local alignment search tool (BLAST) (Table 6). Genes encoding each of the transaminases were synthesized, expressed in, and evaluated for catalytic activity on 6-aminocaproic acid (6ACA) and 7-aminobutyric acid (GABA) using an enzyme-coupled assay.

The genes encoding the TA enzyme candidates of Table 6 were cloned into a low copy number vector under a constitutive promoter and the constructs were transformed into E. coli using standard techniques. Transformants were cultured in LB medium in the presence of antibiotic overnight at 35° C., after which the cells were spun down at 15,000×g at room temperature. To make lysates, the supernatants were removed and E. coli cells expressing the TA gene were resuspended in a chemical lysis solution containing lysozyme, nuclease, and 10 mM DTT. Lysates were used immediately.

The transaminase assay solution contained 0. 1 M Tris-HCl, pH 8. 0; 0. 3 mM 6ACA, 0. 3 mM GABA, 0.3 mM HMD, or 20 mM Ala; 0. 1 mM γ-ketoglutarate; 1 mM NAD; and 50 U/mL glutamate dehydrogenase. The assay was initiated by adding the TA lysate and conducted at room temperature. Activity was monitored by an increase in absorbance of NADH at 340 nm relative to a lysate that contained no TA. The linear rate was calculated as Δabs/min. Rates >100 were designated as (++), rates between 1-100 were designated as (+), and rates with little to no activity were a designated (−). ND=not determined.

Table 6 shows that TA homologs 1, 3, 4, 5, 9, 12, 26, 27, 30, 31, 38, 50, 64, 74, 78, 79, 81, 91, 106, 108, and 116 have the highest activity levels on 6ACA.

TABLE 6
Transaminase Candidate Genes

| | | | | | | In vivo |
|---|---|---|---|---|---|---|
| Homolog | | | | Active | Active | 6ACA |
| # | Organism | SEQ ID NO: | Accession No. | 6ACA | GABA | Production |
| 1 | Achromobacter | SEQ ID NO: 1 | WP_013395096.1 | ++ | ++ | + |
| | xylosoxidans | | | | | |
| 2 | Metagenomic | | HAC67230.1 | − | ++ | |
| 3 | Metagenomic | SEQ ID NO: 3 | WP_100856578.1 | ++ | ++ | + |
| 4 | Metagenomic | SEQ ID NO: 4 | WP_025513437.1 | ++ | ++ | |
| 5 | Metagenomic | SEQ ID NO: 5 | WP_056075635.1 | ++ | ++ | |
| 6 | Metagenomic | | WP_017521342.1 | + | ++ | |
| 7 | Metagenomic | | WP_017201530.1 | ND | ND | |
| 8 | Metagenomic | | WP_059316136.1 | + | ++ | |
| 9 | Metagenomic | SEQ ID NO: 9 | WP_024817707.1 | ++ | ++ | |
| 10 | Metagenomic | | WP_093015681.1 | + | ++ | |
| 11 | Metagenomic | | WP_069902234.1 | − | ++ | |
| 12 | Metagenomic | SEQ ID NO: 12 | WP_009357183.1 | ++ | ++ | |
| 13 | Paenarthrobacter | SEQ ID NO: 13 | SEQ ID NO: 13 | + | ++ | + |
| | aurescens | | | | | |
| | TC1 | | | | | |
| 14 | Metagenomic | | WP_052967527.1 | − | ++ | |
| 15 | Metagenomic | SEQ ID NO: 15 | | + | ++ | |
| 16 | Metagenomic | | WP_043819139.1 | − | + | |
| 17 | Metagenomic | | WP_062073360.1 | + | ++ | |
| 18 | Metagenomic | | WP_015937959.1 | + | ++ | |
| 19 | Metagenomic | | WP_078774761.1 | + | ++ | |
| 20 | Metagenomic | | WP_046815379.1 | + | ++ | |
| 21 | Metagenomic | | WP_090288152.1 | − | + | |
| 22 | Metagenomic | | WP_056075635.1 | ND | ND | |
| 23 | Metagenomic | | WP_003220503.1 | − | − | |
| 24 | Metagenomic | | WP_003255866.1 | − | + | |
| 25 | Metagenomic | | WP_104171925.1 | − | + | |
| 26 | Metagenomic | SEQ ID NO: 26 | WP_123429804.1 | ++ | ++ | + |
| 27 | Metagenomic | SEQ ID NO: 27 | WP_030536079.1 | ++ | ++ | + |
| 28 | Metagenomic | | WP_040078233.1 | + | ++ | |
| 29 | Metagenomic | | EQM68262.1 | − | − | |
| 30 | Metagenomic | SEQ ID NO: 30 | WP_026539700.1 | ++ | ++ | |
| 31 | Metagenomic | SEQ ID NO: 31 | WP_012250302.1 | ++ | ++ | + |
| 32 | Metagenomic | | WP_007983072.1 | − | − | |
| 33 | Metagenomic | | WP_045059477.1 | − | + | |
| 34 | Metagenomic | | WP_093432282.1 | − | ++ | |
| 35 | Metagenomic | | WP_125050266.1 | − | ++ | |
| 36 | Metagenomic | SEQ ID NO:36 | | − | ++ | |
| 37 | Metagenomic | | WP_134929664.1 | − | − | |
| 38 | Metagenomic | SEQ ID NO: 38 | WP_056075635.1 | ++ | ++ | |
| 39 | Metagenomic | | WP_065926318.1 | − | − | |
| 40 | Metagenomic | | WP_123366550.1 | − | − | |
| 41 | Metagenomic | | WP_019473946.1 | − | ++ | |
| 42 | Metagenomic | | WP_065926966.1 | − | ++ | |
| 43 | Metagenomic | | WP_121733815.1 | − | ++ | |
| 44 | Metagenomic | | WP_015937959.1 | − | − | |
| 45 | Metagenomic | | WP_071291640.1 | + | + | |
| 46 | Metagenomic | | WP_111045681.1 | − | − | |
| 47 | Metagenomic | | WP_056737712.1 | + | ++ | |
| 48 | Metagenomic | | WP_042650496.1 | − | ++ | |
| 49 | Metagenomic | | WP_106886181.1 | + | ++ | |
| 50 | Metagenomic | SEQ ID NO: 50 | WP_056563276.1 | ++ | ++ | + |
| 51 | Metagenomic | | WP_017197131.1 | + | ++ | |
| 52 | Metagenomic | SEQ ID NO: 52 | WP_099790225.1 | + | ++ | + |
| 53 | Metagenomic | | WP_048393168.1 | − | + | |
| 54 | Metagenomic | | WP_123366550.1 | − | ++ | |
| 55 | Metagenomic | | WP_017391017.1 | + | ++ | |
| 56 | Metagenomic | SEQ ID NO: 56 | | + | ++ | |
| 57 | Metagenomic | | WP_056075635.1 | − | − | |
| 58 | Metagenomic | | WP_054597933.1 | − | − | |
| 59 | Metagenomic | | WP_095746204.1 | − | − | |
| 60 | Metagenomic | | WP_003220503.1 | − | − | |
| 61 | Metagenomic | | WP_017197131.1 | − | − | |
| 62 | Metagenomic | | WP_052967527.1 | − | ++ | |
| 63 | Metagenomic | | WP_021472307.1 | − | − | |
| 64 | Arthrobacter | SEQ ID NO: 64 | WP_055239729.1 | ++ | ++ | + |
| | sp. Edens01 | | | | | |
| 65 | Arthrobacter | | WP_074699490.1 | − | − | |
| | crytallopoietes | | | | | |
| 66 | Arthrobacter | | WP_079941468.1 | − | ++ | |
| | sp. KI72 | | | | | |
| 67 | Arthrobacter | | WP_087028133.1 | + | ++ | |
| | agilis | | | | | |
| 68 | Metagenomic | | WP_119966131.1 | + | ND | |
| 69 | Metagenomic | | RIK89039.1 | + | ND | |
| 70 | Metagenomic | | WP_016152939.1 | − | ND | |
| 71 | Metagenomic | | WP_089085686.1 | + | ND | |
| 72 | Metagenomic | | WP_054094475.1 | − | ND | |
| 73 | Metagenomic | | WP_105516444.1 | + | ++ | |
| 74 | Metagenomic | SEQ ID NO: 74 | WP_123787193.1 | ++ | ++ | |
| 75 | Metagenomic | | WP_088966344.1 | − | ND | |
| 76 | Metagenomic | | WP_119775651.1 | + | ND | |
| 77 | Metagenomic | SEQ ID NO: 77 | | + | ++ | |
| 78 | Metagenomic | SEQ ID NO: 78 | WP_011748622.1 | ++ | ND | |
| 79 | Metagenomic | SEQ ID NO: 79 | WP_111824632.1 | ++ | ++ | |
| 80 | Metagenomic | | HAU57754.1 | + | ND | |
| 81 | Metagenomic | SEQ ID NO: 81 | SHM39441.1 | ++ | ++ | |
| 82 | Metagenomic | | WP_043683590.1 | + | ND | |
| 83 | Metagenomic | SEQ ID NO: 83 | | − | ND | |
| 84 | Metagenomic | | WP_121579846.1 | + | ND | |
| 85 | Metagenomic | | WP_101193102.1 | + | ND | |
| 86 | Metagenomic | | WP_012249988.1 | + | ++ | |
| 87 | Metagenomic | | WP_119775651.1 | − | ND | |
| 88 | Metagenomic | | OGB44516.1 | + | ND | |
| 89 | Metagenomic | | WP_102820733.1 | + | ND | |
| 90 | Metagenomic | | SHM39441.1 | + | ++ | |
| 91 | Metagenomic | SEQ ID NO: 91 | SHM39441.1 | ++ | ++ | |
| 92 | Metagenomic | | WP_088966344.1 | + | ++ | |
| 93 | Metagenomic | | RIK89039.1 | + | ND | |
| 94 | Metagenomic | | WP_043683590.1 | + | ND | |
| 95 | Metagenomic | | WP_137155920.1 | + | ND | |
| 96 | Metagenomic | | WP_099059309.1 | + | ++ | |
| 97 | Metagenomic | | WP_101193102.1 | ND | ND | |
| 98 | Metagenomic | | WP_043683590.1 | + | ND | |
| 99 | Metagenomic | | WP_062685190.1 | + | ++ | |
| 100 | Metagenomic | | WP_081768742.1 | + | ND | |
| 101 | Metagenomic | | ODT51733.1 | + | ND | |
| 102 | Metagenomic | | WP_104959793.1 | + | ND | |
| 103 | Metagenomic | | WP_043683590.1 | + | ND | |
| 104 | Metagenomic | | HAU57754.1 | + | ND | |
| 105 | Metagenomic | | WP_009615038.1 | − | ND | |
| 106 | Metagenomic | SEQ ID NO: 106 | SHM39441.1 | ++ | ++ | |
| 107 | Metagenomic | | WP_056323744.1 | + | ++ | |
| 108 | Metagenomic | SEQ ID NO: 108 | WP_056323744.1 | ++ | ++ | |
| 109 | Metagenomic | | WP_088966344.1 | + | ++ | |
| 110 | Metagenomic | | SHM39441.1 | + | ND | |
| 111 | Metagenomic | | WP_125868825.1 | + | ND | |
| 112 | Metagenomic | SEQ ID NO: 112 | | + | ND | |
| 113 | Metagenomic | | WP_124644434.1 | + | ND | |
| 114 | Metagenomic | | WP_101193102.1 | + | ND | |
| 115 | Metagenomic | | WP_057067610.1 | + | ND | |
| 116 | Metagenomic | SEQ ID NO: 116 | ODT32214.1 | ++ | ++ | |
| 117 | Metagenomic | | WP_136961719.1 | + | ND | |
| 118 | Metagenomic | | WP_093109347.1 | − | ND | |
| 119 | Metagenomic | | WP_043683590.1 | + | ND | |
| 120 | Metagenomic | | WP_043683590.1 | − | ND | |
| 121 | Metagenomic | | WP_008955628.1 | + | ND | |
| 122 | Metagenomic | | SHM39441.1 | + | ND | |
| 123 | Metagenomic | | WP_003832711.1 | + | ND | |
| 124 | Metagenomic | | WP_116003699.1 | + | ND | |
| 125 | Metagenomic | | WP_120809350.1 | − | ND | |
| 126 | Metagenomic | | WP_026133539.1 | + | ++ | |
| 127 | Metagenomic | | WP_119775651.1 | + | ND | |
| 128 | Metagenomic | | SHM39441.1 | + | ++ | |
| 129 | Metagenomic | | WP_043683590.1 | + | ND | |
| 130 | Metagenomic | | WP_015475018.1 | − | ND | |
| 131 | Metagenomic | | WP_047290758.1 | ND | ND | |
| 132 | Metagenomic | | WP_116810265.1 | − | ND | |
| 133 | Saccharomyces | SEQ ID NO: 133 | NP 011533.3 | + | ++ | |
| | cerevisiae | | | | | |
| 134 | Clostridium | SEQ ID NO: 134 | WP_027096390.1 | + | ++ | |
| | viride | | | | | |
| 135 | Gulosibacter | SEQ ID NO: 135 | WP_026937197.1 | + | ++ | |
| | molinativorax | | | | | |
| 136 | Kocuria | SEQ ID NO: 136 | WP_012399017.1 | ++ | ++ | |
| | rhizophila | | | | | |
| 137 | Arthrobacter | SEQ ID NO: 137 | ABK04473.1 | ++ | ++ | |
| | sp. FB24 | | | | | |
| 138 | Pseudomonas | SEQ ID NO: 138 | WP_009615038.1 | + | + | |
| | sp. AAC | | | | | |
| 139 | Pseudomonas | SEQ ID NO: 139 | WP_043861306.1 | + | + | |
| | putida KG-4 | | | | | |
| 140 | Alcaligenes | SEQ ID NO: 140 | WP_006225725.1 | + | + | |
| | A. faecalis | | | | | |

### Example 2. In Vivo Assays of Transaminase Homologs

To test the activity of transaminase homologs in vivo, genes encoding selected transaminases were transformed into a strain of E. coli that also included introduced genes encoding 1) a 3-oxoadipyl-CoA thiolase (Thl), 2) a 3-oxoadipyl-CoA dehydrogenase (Hbd), 3) a 3-oxoadipyl-CoA dehydratratase (“crotonase” or Crt), 4) a 5-carboxy-2-pentenoyl-CoA reductases (Ter); and 5) an aldehyde dehydrogenase (Ald). The Thl, Hbd, Crt, Ter, Ald genes are reported in U.S. Pat. No. 8,377,680 (e.g., Example 8, which is incorporated by reference in its entirety). These genes are introduced in an E. coli strain included all of the pathway enzymes necessary for producing 6-aminocaproate (6ACA), with the exception of the TA enzyme.

The vectors for expressing the TA genes were transformed into the Thl/Hbd/Crt/Ter/Ald E. coli strain and transformants were tested for 6ACA production. The engineered E. coli cells were fed 2% glucose in minimal media, and after an 18 hour incubation at 35° C., the cells were harvested, and the supernatants were evaluated by analytical IPLC or standard LC/MS analytical method for 6ACA production. As shown in Table 6, expression of genes encoding the TA enzymes in E. coli that included Thl, Hbd, Crt, Ter, and Ald genes resulted in 6ACA production by these strains.

### Example 3. Transaminase Variants

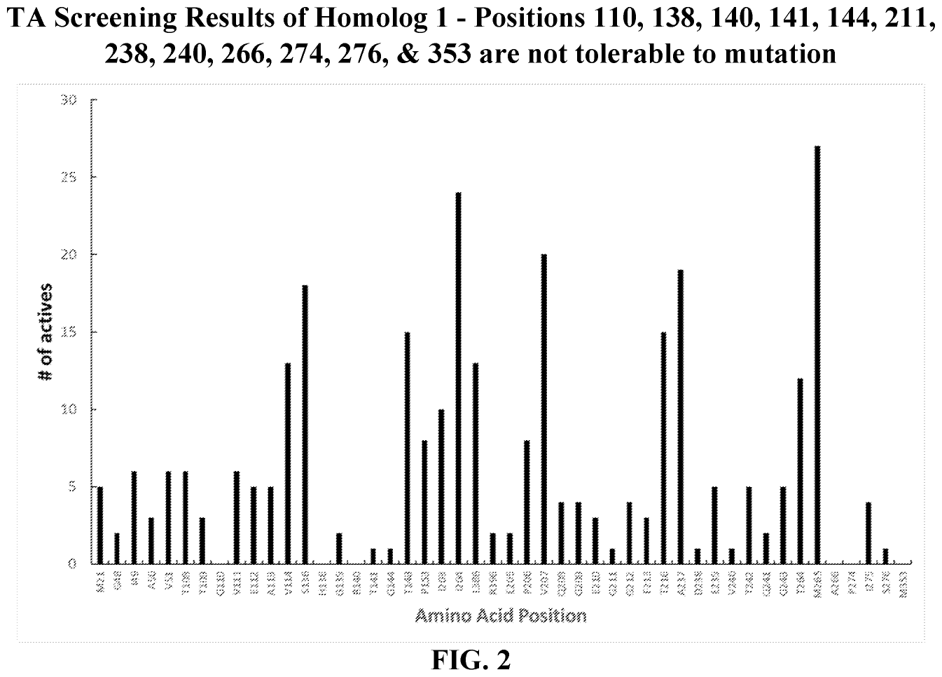

Amino acid positions were identified for mutations in the Homolog 1 TA of Achromobacter xylosoxidans and encoded by SEQ ID NO:1 by examination of the crystal structure of the protein, and a gene encoding SEQ ID NO:1 was subjected to saturation mutagenesis at selected amino acid positions. The resulting variants were tested for activity in the lysate assay as described in Example 1. Results for the TA SEQ ID NO: 1 are shown in FIG. 2. FIG. 2 shows which positions of SEQ ID NO: 1 are important for activity.

Variants were generated by mutating the gene encoding the TA enzyme (SEQ ID NO: 1) at amino acid positions for V114, S136, T148, P153, I203, I204, P206, V207, V111, T216, A237, T264, M265 and L386 as well as the codons for G19, C22, D70, R94, D99, T109, E112, A113, F137, G144, I149, K150, Y154, S178, L186, Q208, L234, T242, A315, K318, R338, G336, L386, V390, A406, S416, and A421. Mutations were made singly and in combination with mutations at other amino acid positions. Mutations at the amino acid positions were made using degenerate primer sequences and PCR, where the altered gene sequence mixtures were transformed into E. coli. Transformants were tested in lysate assays as described in Example 1. Clones that provided lysates showing activity higher than wild type controls in the assays were retested in triplicate lysate assays. Clones that continued to demonstrate higher than wild type activity were then prepped for sequence analysis of the TA genes they contained.

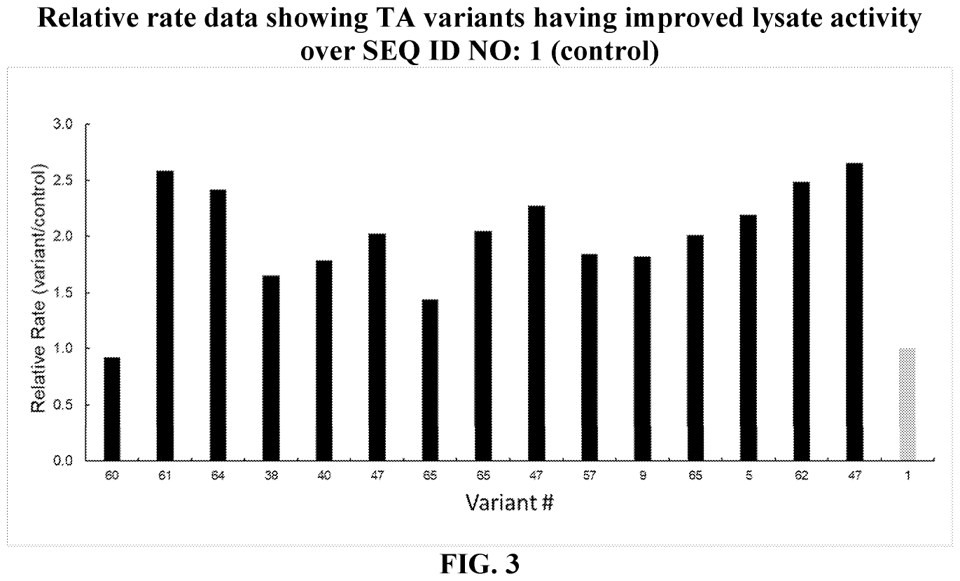

Table 7 and FIG. 3 provide the mutations found in the variant TA gene sequences of the active clones. Variants demonstrating higher than wild type activity, denoted “+” or “++”, included single mutations and combinatorial (multiple) mutations in the TA gene. Table 7 shows that multiple variants demonstrated greater activity than the wild type TA (SEQ ID NO:1), with mutations at amino acid positions Vi 14, S136, T148, P153, I203, I204, P206, V207, V111, T216, A237, T264, M265, L386, G19, C22, D70, R94, D99, T109, E112, A113, F137, G144, 1149, K150, Y154, S178, L186, Q208, L234, T242, A315, K318, R338, G336, L386, V390, A406, S416, A421, G17, M21, A50, A76, Y77, Q78, 179, G84, F107, T108, K119, G139, M142, A152, P153, E205, G209, G211, D238, M285, A290, G291, G292, L293, Y297, M353, S387, S388, and G392 (positions identified with respect to SEQ ID NO:1) resulting in multiple variants with higher activity than the wild type TA from which they were derived.

In addition, several of the variants were assayed using 20 mM alanine as the substrate instead of 6ACA or GABA.

TABLE 7
Transaminase Variants

| | | | | | In vivo |
|---|---|---|---|---|---|
| Variant | | Active | Active | Active | 6ACA |
| # | Amino acid substitutions to SEQ ID NO: 1 | 6ACA | GABA | Ala | Production |
| 1 | A113V | + | ++ | ND | |
| 2 | E112K | − | − | ND | |
| 3 | I49V | + | ++ | ND | |
| 4 | T264S | + | ++ | ND | |
| 5 | G19R C22S D70N L186V K318M G336S | + | ++ | ND | |
| | S416Y | | | | |
| 6 | V111A | + | ++ | ND | |
| 7 | I203L | + | ++ | ND | |
| 8 | T148I | + | ++ | ND | |
| 9 | G19R D70N D99E L186V K318M G336S | + | ++ | ND | |
| | S416N | | | | |
| 10 | T109S | + | ++ | ND | |
| 11 | T148V | + | ++ | ND | |
| 12 | S136A | ++ | ++ | ND | |
| 13 | Q208R | + | ++ | ND | |
| 14 | L386V | + | ++ | ND | |
| 15 | G144C | + | ++ | ND | |
| 16 | I49V S136A T148I | + | ++ | ND | |
| 17 | S136C | + | ++ | ND | |
| 18 | S136G | + | ++ | ND | |
| 19 | I204K | + | ++ | ND | |
| 20 | M265C | + | ++ | ND | |
| 21 | V207E | + | ++ | ND | |
| 22 | S136A T148I V207E | ++ | ++ | ND | |
| 23 | V207T | + | ++ | ND | |
| 24 | I204Q | + | ++ | ND | |
| 25 | I204T | + | ++ | ND | |
| 26 | L386C | + | ++ | ND | |
| 27 | M265N | + | ++ | ND | |
| 28 | G19R D70N L186V K318M G336S S416Y | + | ++ | ND | |
| 29 | A237T | + | ++ | ND | |
| 30 | A237D | + | ++ | ND | |
| 31 | A237V | + | ++ | ND | |
| 32 | A237G | + | ++ | ND | |
| 33 | A237S | ++ | ++ | ND | |
| 34 | G243C | + | ++ | ND | |
| 35 | I49V S136A T148I V207E | ++ | ++ | ND | |
| 36 | M265A | + | ++ | ND | |
| 37 | T216V | + | ++ | ND | |
| 38 | G19R D70N F133L L186V K318M G336S S416Y | + | ++ | ND | |
| 39 | T216A | + | ++ | ND | |
| 40 | G19R C22S D70N L186V K318M G336S S416Y | + | ++ | ND | |
| 41 | T216C | + | ++ | ND | |
| 42 | T242A | + | ++ | ND | |
| 43 | T264P | + | ++ | ND | |
| 44 | F137W Y154N | + | ++ | ND | |
| 45 | Y154N | + | ++ | ND | |
| 46 | Y154I | + | ++ | ND | |
| 47 | G19R D70N L186V K318M G336S S416D | + | ++ | ND | |
| 48 | Y154L | + | ++ | ND | |
| 49 | Y154V | + | ++ | ND | |
| 50 | Y154F | + | ++ | ND | |
| 51 | Y154T | + | ++ | ND | |
| 52 | Y154C | + | + | ND | |
| 53 | Y154M | + | ++ | ND | |
| 54 | S136A T148I | ++ | ++ | ND | |
| 55 | F137T Y154K | − | ND | + | |
| 56 | F137I | − | ND | + | |
| 57 | G19R D70N K150R L186V L234I K318M G336S | + | ++ | ND | |
| | V390D S416N | | | | |
| 58 | T148D | + | ND | + | |
| 59 | L386A | + | ND | + | |
| 60 | R94H S178T A315V R338L | + | ++ | ND | |
| 61 | K226T R338L | + | ++ | ND | |
| 62 | R338L | + | ++ | ND | |
| 63 | L386C | + | ND | + | |
| 64 | G19R D70N L186V K318M G336S A406E | + | ++ | ND | |
| | S416Y | | | | |
| 65 | G19R D70N L186V K318M G336S L386P S416Y | + | ++ | ND | |
| | A421E | | | | |
| 66 | G19R D70N A76G L186V K318M G336S L386P | ++ | ++ | ND | |
| | S416Y A421E | | | | |
| 67 | G19R D70N A76Q L186V K318M G336S L386P | ++ | ++ | ND | |
| | S416Y A421E | | | | |
| 68 | G19R D70N G139E L186V K318M G336S | + | ++ | ND | |
| | L386P S416Y A421E | | | | |
| 69 | G19R D70N L186V G291S K318M G336S L386P | ++ | ++ | ND | |
| | S416Y A421E | | | | |
| 70 | G19R D70N L186V G292C K318M G336S | ++ | ++ | ND | |
| | L386P S416Y A421E | | | | |
| 71 | G19R I49Y D70N L186V K318M G336S L386P | ++ | ++ | ND | |
| | S416Y A421E | | | | |
| 72 | G19R D70N I79V L186V K318M G336S L386P | ++ | ++ | ND | |
| | S416Y A421E | | | | |
| 73 | G19R D70N K119Y L186V K318M G336S | + | ++ | ND | |
| | L386P S416Y A421E | | | | |
| 74 | G19R D70N L186V K318M G336S L386P L293C | ++ | ++ | ND | |
| | S416Y A421E | | | | |
| 75 | G19R D70N L186V K318M G336S L386P | ++ | ++ | ND | |
| | L293M S416Y A421E | | | | |
| 76 | G19R D70N M142S L186V K318M G336S | ++ | ++ | ND | |
| | L386P S416Y A421E | | | | |
| 77 | G19R D70N M142C L186V K318M G336S | ++ | ++ | ND | |
| | L386P S416Y A421E | | | | |
| 78 | G19R D70N M142Y L186V K318M G336S | ++ | ++ | ND | |
| | L386P S416Y A421E | | | | |
| 79 | G19R D70N P153D L186V K318M G336S L386P | ++ | ++ | ND | |
| | S416Y A421E | | | | |
| 80 | G19R D70N Q78V L186V K318M G336S L386P | ++ | ++ | ND | |
| | S416Y A421E | | | | |
| 81 | G19R D70N Q78A L186V K318M G336S L386P | ++ | ++ | ND | |
| | S416Y A421E | | | | |
| 82 | G19R D70N Q78Y L186V K318M G336S L386P | ++ | ++ | ND | |
| | S416Y A421E | | | | |
| 83 | G19R D70N Q78N L186V K318M G336S L386P | ++ | ++ | ND | |
| | S416Y A421E | | | | |
| 84 | G19R D70N V111S L186V K318M G336S L386P | ++ | ++ | ND | |
| | S416Y A421E | | | | |
| 85 | G19R D70N V111A L186V K318M G336S | ++ | ++ | ND | |
| | L386P S416Y A421E | | | | |
| 86 | G19R D70N Y154W L186V K318M G336S | + | ++ | ND | |
| | L386P S416Y A421E | | | | |
| 87 | G19R D70N Y154F L186V K318M G336S L386P | ++ | ++ | ND | |
| | S416Y A421E | | | | |
| 88 | G19R D70N Y77F L186V K318M G336S L386P | ++ | ++ | ND | |
| | S416Y A421E | | | | |
| 89 | G19R D70N I79V L186V K318M G336S L386P | ++ | ++ | ND | |
| | S416Y A421E | | | | |
| 90 | G19R D70N Q78N L186V K318M G336S L386P | ++ | ++ | + | + |
| | S416Y A421E | | | | |
| 91 | G19R D70N Q78N I79V L186V K318M G336S | ++ | ++ | ND | |
| | L386P S416Y A421E | | | | |
| 92 | G19R D70N Q78Y L186V K318M G336S L386P | ++ | ++ | ND | |
| | S416Y A421E | | | | |
| 93 | G19R D70N Q78Y I79V L186V K318M G336S | ++ | ++ | ND | |
| | L386P S416Y A421E | | | | |
| 94 | G19R D70N A76G L186V K318M G336S L386P | ++ | ++ | ND | |
| | S416Y A421E | | | | |
| 95 | G19R D70N A76G I79V L186V K318M G336S | ++ | ++ | ND | |
| | L386P S416Y A421E | | | | |
| 96 | G19R D70N A76G Q78N L186V K318M G336S | ++ | ++ | ND | |
| | L386P S416Y A421E | | | | |
| 97 | G19R D70N A76G Q78N I79V L186V K318M | ++ | ++ | ND | |
| | G336S L386P S416Y A421E | | | | |
| 98 | G19R D70N A76G Q78Y L186V K318M G336S | ++ | ++ | ND | |
| | L386P S416Y A421E | | | | |
| 99 | G19R D70N A76G Q78Y I79V L186V K318M | ++ | ++ | ND | |
| | G336S L386P S416Y A421E | | | | |
| 100 | G19R D70N A76G L186V K318M G336S L386P | ++ | ++ | ND | |
| | S416Y A421E | | | | |
| 101 | G19R D70N A76Q I79V L186V K318M G336S | ++ | ++ | ND | |
| | L386P S416Y A421E | | | | |
| 102 | G19R D70N A76Q Q78N L186V K318M G336S | ++ | ++ | ND | |
| | L386P S416Y A421E | | | | |
| 103 | G19R D70N A76Q Q78N I79V L186V K318M | ++ | ++ | + | + |
| | G336S L386P S416Y A421E | | | | |
| 104 | G19R D70N A76Q Q78Y L186V K318M G336S | − | ++ | ND | |
| | L386P S416Y A421E | | | | |
| 105 | G19R D70N A76Q Q78Y I79V L186V K318M | + | ++ | ND | |
| | G336S L386P S416Y A421E | | | | |
| 106 | G19R D70N V111A L186V K318M G336S | ++ | ++ | ND | |
| | L386P S416Y A421E | | | | |
| 107 | G19R D70N I79V V111A L186V K318M G336S | ++ | ++ | ND | |
| | L386P S416Y A421E | | | | |
| 108 | G19R D70N Q78N V111A L186V K318M G336S | ++ | ++ | ND | |
| | L386P S416Y A421E | | | | |
| 109 | G19R D70N Q78N I79V V111A L186V K318M | ++ | ++ | ND | |
| | G336S L386P S416Y A421E | | | | |
| 110 | G19R D70N Q78Y V111A L186V K318M G336S | ++ | ++ | + | + |
| | L386P S416Y A421E | | | | |
| 111 | G19R D70N Q78Y I79V V11A L186V K318M | ++ | ++ | ND | |
| | G336S L386P S416Y A421E | | | | |
| 112 | G19R D70N A76G V111A L186V K318M G336S | ++ | ++ | ND | |
| | L386P S416Y A421E | | | | |
| 113 | G19R D70N A76G I79V V111A L186V K318M | ++ | ++ | ND | |
| | G336S L386P S416Y A421E | | | | |
| 114 | G19R D70N A76G Q78N V111A L186V K318M | ++ | ++ | ND | |
| | G336S L386P S416Y A421E | | | | |
| 115 | G19R D70N A76G Q78N I79V V111A L186V | ++ | ++ | ND | |
| | K318M G336S L386P S416Y A421E | | | | |
| 116 | G19R D70N A76G Q78Y V111A L186V K318M | ++ | ++ | ND | |
| | G336S L386P S416Y A421E | | | | |
| 117 | G19R D70N A76G Q78Y I79V V11A L186V | ++ | ++ | ND | |
| | K318M G336S L386P S416Y A421E | | | | |
| 118 | G19R D70N A76Q V111A L186V K318M G336S | ++ | ++ | ND | |
| | L386P S416Y A421E | | | | |
| 119 | G19R D70N A76Q I79V V111A L186V K318M | ++ | ++ | + | + |
| | G336S L386P S416Y A421E | | | | |
| 120 | G19R D70N A76Q Q78N V111A L186V K318M | ++ | ++ | ND | |
| | G336S L386P S416Y A421E | | | | |
| 121 | G19R D70N A76Q Q78N I79V V111A L186V | ++ | ++ | ND | |
| | K318M G336S L386P S416Y A421E | | | | |
| 122 | G19R D70N A76Q Q78Y V111A L186V K318M | + | ++ | ND | |
| | G336S L386P S416Y A421E | | | | |
| 123 | G19R D70N A76Q Q78Y I79V V11A L186V | + | ++ | ND | |
| | K318M G336S L386P S416Y A421E | | | | |
| 124 | A76Q Q78N I79V L386P | + | + | + | + |
| 125 | G19R D70N A76Q Q78N I79V S136A M142I | ++ | ++ | ND | |
| | L186I A290L K318M G336S L386P V390A | | | | |
| | S416Y A421E | | | | |
| 126 | G19R D70N A76Q Q78N I79V K318M G336S | ++ | ++ | ND | + |
| | L386P V390L S416Y A421E | | | | |
| 127 | G19R D70N A76Q Q78N I79V K318M G336S | ++ | ++ | ND | |
| | L386P V390L S416Y A421E | | | | |
| 128 | G19R D70N A76Q Q78N I79V S136A L186V | ++ | ++ | ND | |
| | Y297F K318M G336S L386V V390A S416Y | | | | |
| | A421E | | | | |
| 129 | G19R D70N A76Q Q78N I79V S136A A152V | ++ | ++ | ND | |
| | L186M A290L Y297F K318M G336S L386I | | | | |
| | S416Y A421E | | | | |
| 130 | G19R D70N A76Q Q78N I79V A152T L186I | ++ | ++ | + | + |
| | Y297F K318M G336S L386V V390L S416Y | | | | |
| | A421E | | | | |
| | G19R D70N A76Q Q78N I79V A152T L186I | | | | |
| | Y297F K318M G336S L386P V390L S416Y | | | | |
| | A421E | | | | |
| 131 | G19R D70N A76Q Q78N I79V S136G A152T | ++ | ++ | ND | |
| | L186M K318M G336S L386V S416Y A421E | | | | |
| 132 | G19R D70N A76Q Q78N I79V A152T L186M | ++ | ++ | ND | |
| | A290L K318M G336S L386V S416Y A421E | | | | |
| 133 | G19R D70N A76Q Q78N I79V S136A A152V | ++ | ++ | ND | |
| | L186M Y297F K318M G336S L386P V390A | | | | |
| | S416Y A421E | | | | |
| 134 | G19R D70N A76Q Q78N I79V S136G A152V | ++ | ++ | ND | |
| | L1861 Y297F K318M G336S L386P S416Y | | | | |
| | A421E | | | | |
| 135 | G19R D70N A76Q Q78N I79V S136G A152T | ++ | ++ | ND | |
| | L186M A290L K318M G336S L386V S416Y | | | | |
| | A421E | | | | |
| 136 | G19R D70N A76Q Q78N I79V S136A A152V | ++ | ++ | + | + |
| | Y297F K318M G336S L386V V390A S416Y | | | | |
| | A421E | | | | |
| 137 | G19R D70N A76Q Q78N I79V S136A L186M | ++ | ++ | ND | |
| | A290L K318M G336S L386I V390L S416Y | | | | |
| | A421E | | | | |
| 138 | G19R D70N A76Q Q78N I79V S136A A152T | ++ | ++ | ND | |
| | L186M K318M G336S L386V S416Y A421E | | | | |
| 139 | G19R D70N A76Q Q78N I79V S136A L186M | ++ | ++ | ND | |
| | A290L K318M G336S L386V S416Y A421E | | | | |
| 140 | G19R D70N A76Q Q78N I79V S136A A152T | ++ | ++ | ND | |
| | L186I A290L K318M G336S L386V V390L | | | | |
| | S416Y A421E | | | | |
| 141 | G19R D70N A76Q Q78N I79V S136A L186I | ++ | ++ | ND | |
| | K318M G336S L386V Y297F V390A S416Y | | | | |
| | A421E | | | | |
| 142 | G19R D70N A76Q Q78N I79V S136A K318M | ++ | ++ | ND | |
| | G336S L386V V390L S416Y A421E | | | | |
| 143 | G19R D70N A76Q Q78N I79V S136A K318M | ++ | ++ | ND | |
| | G336S L386V V390A S416Y A421E | | | | |
| 144 | G19R D70N A76Q Q78N I79V S136G K318M | ++ | ++ | ND | |
| | G336S Y297F L386P S416Y A421E | | | | |
| 145 | G19R D70N A76Q Q78N I79V A152T A290L | ++ | ++ | ND | |
| | K318M G336S Y297F L386P S416Y A421E | | | | |
| 146 | G19R D70N A76Q Q78N I79V A152T A290L | ++ | ++ | ND | |
| | K318M G336S Y297F L386P S416Y A421E | | | | |
| 147 | G19R D70N A76Q Q78N I79V S136A A152T | ++ | ++ | ND | |
| | L186A A290L K318M G336S Y297F L386P | | | | |
| | V390A S416Y A421E | | | | |
| 148 | G19R D70N A76Q Q78N I79V S136G A152V | ++ | ++ | ND | |
| | L186M A290L K318M G336S Y297F L386P | | | | |
| | S416Y A421E | | | | |
| 149 | G19R D70N A76Q Q78N I79V K318M G336S | ++ | ++ | + | + |
| | Y297F L386V S416Y A421E | | | | |
| 150 | G17A G19R D70N A76Q Q78N I79V L186V | + | + | ND | |
| | K318M G336S L386P S416Y A421E | | | | |
| 151 | G19K D70N A76Q Q78N I79V L186V K318M | + | + | ND | |
| | G336S L386P S416Y A421E | | | | |
| 152 | G19Q D70N A76Q Q78N I79V L186V K318M | + | + | ND | |
| | G336S L386P S416Y A421E | | | | |
| 153 | M21Q G19R D70N A76Q Q78N I79V L186V | + | + | ND | |
| | K318M G336S L386P S416Y A421E | | | | |
| 154 | M21Q M285I G19R D70N A76Q Q78N I79V | + | + | ND | |
| | L186V K318M G336S L386P S416Y A421E | | | | |
| 155 | G17S G19R D70N A76Q Q78N I79V L186V | + | + | ND | |
| | K318M G336S L386P S416Y A421E | | | | |
| 156 | G17Y G19R D70N A76Q Q78N I79V L186V | − | + | ND | |
| | K318M G336S L386P S416Y A421E | | | | |
| 157 | C22M G19R D70N A76Q Q78N I79V L186V | − | + | ND | |
| | K318M G336S L386P S416Y A421E | | | | |
| 158 | C22Y G19R D70N A76Q Q78N I79V L186V | + | + | ND | |
| | K318M G336S L386P S416Y A421E | | | | |
| 159 | N70A G19R D70N A76Q Q78N I79V L186V | + | + | ND | |
| | K318M G336S L386P S416Y A421E | | | | |
| 160 | N70Y G19R D70N A76Q Q78N I79V L186V | − | + | ND | |
| | K318M G336S L386P S416Y A421E | | | | |
| 161 | G19R A76Q Q78N I79V L186V K318M G336S | + | + | ND | |
| | L386P S416Y A421E | | | | |
| 162 | G19R D70C A76Q Q78N I79V L186V K318M | + | + | ND | |
| | G336S L386P S416Y A421E | | | | |
| 163 | A50N G19R D70N A76Q Q78N I79V L186V | − | + | ND | |
| | K318M G336S L386P S416Y A421E | | | | |
| 164 | F107M G19R D70N A76Q Q78N I79V L186V | + | + | ND | |
| | K318M G336S L386P S416Y A421E | | | | |
| 165 | F107S G19R D70N A76Q Q78N I79V L186V | − | + | ND | |
| | K318M G336S L386P S416Y A421E | | | | |
| 166 | F107Q G19R D70N A76Q Q78N I79V L186V | + | + | ND | |
| | K318M G336S L386P S416Y A421E | | | | |
| 167 | T108Q G19R D70N A76Q Q78N I79V L186V | − | − | ND | |
| | K318M G336S L386P S416Y A421E | | | | |
| 168 | T108A G19R D70N A76Q Q78N I79V L186V | − | + | ND | |
| | K318M G336S L386P S416Y A421E | | | | |
| 169 | E112M G19R D70N A76Q Q78N I79V L186V | − | + | ND | |
| | K318M G336S L386P S416Y A421E | | | | |
| 170 | S136D G19R D70N A76Q Q78N I79V L186V | + | + | ND | |
| | K318M G336S L386P S416Y A421E | | | | |
| 171 | S136G G19R D70N A76Q Q78N I79V L186V | ++ | ++ | ND | |
| | K318M G336S L386P S416Y A421E | | | | |
| 172 | S136A G19R D70N A76Q Q78N I79V L186V | + | ++ | ND | |
| | K318M G336S L386P S416Y A421E | | | | |
| 173 | A152M G19R D70N A76Q Q78N I79V L186V | + | ++ | ND | |
| | K318M G336S L386P S416Y A421E | | | | |
| 174 | A152V G19R D70N A76Q Q78N I79V L186V | + | ++ | ND | |
| | K318M G336S L386P S416Y A421E | | | | |
| 175 | A152C G19R D70N A76Q Q78N I79V L186V | + | ++ | ND | |
| | K318M G336S L386P S416Y A421E | | | | |
| 176 | A152L G19R D70N A76Q Q78N I79V L186V | + | ++ | ND | |
| | K318M G336S L386P S416Y A421E | | | | |
| 177 | A152T G19R D70N A76Q Q78N I79V L186V | + | ++ | ND | |
| | K318M G336S L386P S416Y A421E | | | | |
| 178 | A152Q G19R D70N A76Q Q78N I79V L186V | + | ++ | ND | |
| | K318M G336S L386P S416Y A421E | | | | |
| 179 | K150H G19R D70N A76Q Q78N I79V L186V | + | + | ND | |
| | K318M G336S L386P S416Y A421E | | | | |
| 180 | G19R D70N A76Q Q78N I79V L186I K318M | + | ++ | ND | |
| | G336S L386P S416Y A421E | | | | |
| 181 | G19R D70N A76Q Q78N I79V L186M K318M | + | ++ | ND | |
| | G336S L386P S416Y A421E | | | | |
| 182 | E205D G19R D70N A76Q Q78N I79V L186V | + | + | ND | |
| | K318M G336S L386P S416Y A421E | | | | |
| 183 | D238E G19R D70N A76Q Q78N I79V L186V | − | + | ND | |
| | K318M G336S L386P S416Y A421E | | | | |
| 184 | D238I G19R D70N A76Q Q78N I79V L186V | − | + | ND | |
| | K318M G336S L386P S416Y A421E | | | | |
| 185 | G84V G19R D70N A76Q Q78N I79V L186V | + | + | ND | |
| | K318M G336S L386P S416Y A421E | | | | |
| 186 | D238M G19R D70N A76Q Q78N I79V L186V | − | + | ND | |
| | K318M G336S L386P S416Y A421E | | | | |
| 187 | G209G G19R D70N A76Q Q78N I79V L186V | + | ++ | ND | |
| | K318M G336S L386P S416Y A421E | | | | |
| 188 | G211N G19R D70N A76Q Q78N I79V L186V | − | + | ND | |
| | K318M G336S L386P S416Y A421E | | | | |
| 189 | T242F G19R D70N A76Q Q78N I79V L186V | − | + | ND | |
| | K318M G336S L386P S416Y A421E | | | | |
| 190 | A290D G19R D70N A76Q Q78N I79V L186V | − | + | ND | |
| | K318M G336S L386P S416Y A421E | | | | |
| 191 | A290K G19R D70N A76Q Q78N I79V L186V | + | + | ND | |
| | K318M G336S L386P S416Y A421E | | | | |
| 192 | A290I G19R D70N A76Q Q78N I79V L186V | + | + | ND | |
| | K318M G336S L386P S416Y A421E | | | | |
| 193 | Y297F G19R D70N A76Q Q78N I79V L186V | + | ++ | ND | |
| | K318M G336S L386P S416Y A421E | | | | |
| 194 | Y297P G19R D70N A76Q Q78N I79V L186V | + | + | ND | |
| | K318M G336S L386P S416Y A421E | | | | |
| 195 | M353N G19R D70N A76Q Q78N I79V L186V | + | + | ND | |
| | K318M G336S L386P S416Y A421E | | | | |
| 196 | G19R D70N A76Q Q78N I79V L186V K318F | + | ++ | ND | |
| | G336S L386P S416Y A421E | | | | |
| 197 | S387Y G19R D70N A76Q Q78N I79V L186V | + | + | ND | |
| | K318M G336S L386P S416Y A421E | | | | |
| 198 | C388A G19R D70N A76Q Q78N I79V L186V | + | + | ND | |
| | K318M G336S L386P S416Y A421E | | | | |
| 199 | G389G G19R D70N A76Q Q78N I79V L186V | + | + | ND | |
| | K318M G336S L386P S416Y A421E | | | | |
| 200 | G392T G19R D70N A76Q Q78N I79V L186V | − | + | ND | |
| | K318M G336S L386P S416Y A421E | | | | |
| 201 | G392N G19R D70N A76Q Q78N I79V L186V | + | + | ND | |
| | K318M G336S L386P S416Y A421E | | | | |
| 202 | V390L G19R D70N A76Q Q78N I79V L186V | + | ++ | ND | |
| | K318M G336S L386P S416Y A421E | | | | |
| 203 | V390D G19R D70N A76Q Q78N I79V L186V | + | + | ND | |
| | K318M G336S L386P S416Y A421E | | | | |
| 204 | V390A G19R D70N A76Q Q78N I79V L186V | + | + | ND | |
| | K318M G336S L386P S416Y A421E | | | | |
| 205 | G19R D70N A76Q Q78N I79V L186V K318M | + | + | ND | |
| | G336S L386P S416W A421E | | | | |
| 206 | G19R D70N A76Q Q78N I79V L186V K318M | + | + | ND | |
| | G336S L386P S416C A421E | | | | |
| 207 | G392A G19R D70N A76Q Q78N I79V L186V | + | + | ND | |
| | K318M G336S L386P S416Y A421E | | | | |
| 208 | G19R D70N A76Q Q78N I79V L186V K318M | + | + | ND | |
| | G336S L386S S416Y A421E | | | | |
| 209 | G19R D70N A76Q Q78N I79V L186V K318M | + | + | ND | |
| | G336S L386P S416Y A421D | | | | |
| 210 | G19R D70N A76Q Q78N I79V L186V K318M | ++ | ++ | ND | |
| | G336S S416Y A421E | | | | |
| 211 | G19R D70N A76Q Q78N I79V L186V K318M | ++ | ++ | ND | |
| | G336S L386V S416Y A421E | | | | |
| 212 | S136A A152V V386I V390A G19R D70N A76Q | ++ | ++ | ND | |
| | Q78N I79V K318M G336S Y297F L386V S416Y | | | | |
| | A421E | | | | |
| 213 | S136A A152T V390L G19R D70N A76Q Q78N | ++ | ++ | ND | |
| | I79V K318M G336S Y297F L386I S416Y A421E | | | | |
| 214 | S136A A152V V186I A290L Y297F G19R D70N | ++ | ++ | ND | |
| | A76Q Q78N I79V K318M G336S Y297F L386V | | | | |
| | S416Y A421E | | | | |
| 215 | S136A V186M Y297F G19R D70N A76Q Q78N | ++ | ++ | ND | |
| | I79V K318M G336S L386V S416Y A421E | | | | |
| 216 | S136A A152V G19R D70N A76Q Q78N I79V | ++ | ++ | ND | |
| | K318M G336S Y297F L386I S416Y A421E | | | | |
| 217 | G19R D70N A76Q Q78N I79V K318M G336S | ++ | ++ | ND | |
| | Y297F L386I S416Y A421E | | | | |
| 218 | V186I V390A G19R D70N A76Q Q78N I79V | ++ | ++ | ND | |
| | K318M G336S Y297F L386I S416Y A421E | | | | |
| 219 | S136A G19R D70N A76Q Q78N I79V K318M | ++ | ++ | ND | |
| | G336S Y297F L386V S416Y A421E | | | | |
| 220 | S136G A152V V186I G19R D70N A76Q Q78N | ++ | ++ | ND | |
| | I79V K318M G336S Y297F L386V S416Y | | | | |
| | A421E | | | | |
| 221 | V186I G19R D70N A76Q Q78N I79V K318M | ++ | ++ | ND | |
| | G336S Y297F L386V S416Y A421E | | | | |
| 222 | S136A A290L V390L G19R D70N A76Q Q78N | ++ | ++ | ND | |
| | I79V K318M G336S Y297F L386V S416Y | | | | |
| | A421E | | | | |
| 223 | S136A V186A G19R D70N A76Q Q78N I79V | ++ | ++ | ND | |
| | K318M G336S Y297F L386V S416Y A421E | | | | |
| 224 | S136A V186M G19R D70N A76Q Q78N I79V | ++ | ++ | ND | |
| | K318M G336S Y297F L386V S416Y A421E | | | | |
| 225 | S136A V186M A290L G19R D70N A76Q Q78N | ++ | ++ | ND | |
| | I79V K318M G336S Y297F L386V S416Y | | | | |
| | A421E | | | | |
| 226 | S136G V186I G19R D70N A76Q Q78N I79V | ++ | ++ | ND | |
| | K318M G336S Y297F L386V S416Y A421E | | | | |
| 227 | S136A A152V A290L G19R D70N A76Q Q78N | ++ | ++ | ND | |
| | I79V K318M G336S Y297F L386V S416Y | | | | |
| | A421E | | | | |
| 228 | S136G V186I G19R D70N A76Q Q78N I79V | ++ | ++ | ND | |
| | K318M G336S Y297F L386V S416Y A421E | | | | |
| 229 | S136G V186M G19R D70N A76Q Q78N I79V | ++ | ++ | ND | |
| | K318M G336S Y297F L386V S416Y A421E | | | | |
| 230 | S136A V186A G19R D70N A76Q Q78N I79V | ++ | ++ | ND | |
| | K318M G336S Y297F L386V S416Y A421E | | | | |
| 231 | S136G V186M G19R D70N A76Q Q78N I79V | ++ | ++ | ND | |
| | K318M G336S Y297F L386V S416Y A421E | | | | |
| 232 | S136A A152V G19R D70N A76Q Q78N I79V | ++ | ++ | ND | |
| | K318M G336S Y297F L386V S416Y A421E | | | | |
| 233 | S136A G19R D70N A76Q Q78N I79V K318M | ++ | ++ | ND | |
| | G336S Y297F L386V S416Y A421E | | | | |

### Example 4. Identification of CAR Homologs with Activity on 6-Aminocaproic Acid (6ACA)

Genes encoding CARs were identified bioinformatically from metagenomic libraries and public databases using a basic local alignment search tool (BLAST) (Table 8). Genes encoding each of the CARs were synthesized, expressed in, and evaluated for catalytic activity on 6-aminocaproic acid (6ACA) and hexanoate (GABA) using an enzyme-coupled assay.

Briefly, to evaluate carboxylic acid reductase (CAR) candidates and variants, an E. coli strain containing an integrated phosphopantetheine transferase strain harboring the CAR plasmid was generated. The strain was inoculated in LB with carbenicillin (100 μg/mL) and grown overnight at 37° C. in a shaking incubator. The overnight culture was diluted into into fresh LB with carbenicillin (100 μg/mL), IPTG (0.5 mM) and cumate (0.2 mM) and grown overnight at 27° C. in a shaking incubator. Cells were collected by centrifugation and frozen at −20° C. until the day of assay.

For in vitro lysate assay, the cell pellet was thawed and resuspended in 0.1 M Tris-HCl, pH 7.0 buffer. The OD600 was measured of cell suspension and each of the candidates were normalized to an OD of 4. Pellets were prepared by centrifugation and the pellet was then lysed with a chemical lysis reagent containing nuclease and lysozyme for 30 minutes at room temperature. This lysate was used to measure the CAR activity and the assay was carried out as follows: aliquot of the crude CAR lysate, desired acid substrate (hexanoate, 6-aminocaproic acid, butyrate, and 4-aminobutyric acid), 1 mM ATP, 0.3 mM NADPH, and 10 mM MgCl₂ were mixed in 0.04 mL of 0.1 M Tris-HCl, pH 7.4 buffer. The kinetics of the reaction was monitored by NADPH oxidation either by fluorescence or absorbance. The rate of CAR activity was determined from the progress curve.

Table 8 shows that CAR homologs corresponding to SEQ TD NOS: 150-165, 168-171, 173-178, 180, 183-185, 187-188, 190-193, 195, 198-200, 202-216, 218-219, 221-230, 232-238, 241-244, 246-249, 251-252 and 255-264 all exhibit activity on 6ACA, hexanoate or both.

TABLE 8
CAR Homologs

| | | SEQ | | | | In vivo |
|---|---|---|---|---|---|---|
| Homolog | | ID | | Active | Active | HMD |
| # | Organism | NO: | Accession No. | 6ACA | Hexanoate | production |
| 1 | Mycolicibacterium | 150 | WP_011728718.1 | + | + | |
| | smegmatis | | | | | |
| | MC2 155 | | | | | |
| 2 | Mycolicibacterium | 151 | AFP42026.1 | + | + | |
| | smegmatis | | | | | |
| | MC2 155 | | | | | |
| 3 | Mycobacterium | 152 | WP_003872682.1 | + | + | |
| | avium | | | | | |
| 4 | Mycobacterium | 153 | WP_003872682.1 | + | +++ | + |
| | avium | | | | | |
| 5 | Mycobacterium | 154 | WP_012393886.1 | + | + | |
| | marinum M | | | | | |
| 6 | Mycobacterium | 155 | ACC41375.1 | + | + | |
| | liflandii | | | | | |
| | 128FXT | | | | | |
| 7 | Mycobacterium | 156 | WP_011799278.1 | + | + | |
| | tuberculosis | | | | | |
| | variant bovis | | | | | |
| | BCG | | | | | |
| 8 | Streptomyces | 157 | WP_012382217.1 | + | + | |
| | griseus subsp. | | | | | |
| | griseus NBRC | | | | | |
| | 13350 | | | | | |
| 9 | Saccharomyces | 158 | NP 009673.1 | + | + | |
| | cerevisiae | | | | | |
| | S288C | | | | | |
| 10 | Cyanobium sp. | 159 | EDY38441.1 | + | + | |
| | PCC 7001 | | | | | |
| 11 | Neurospora | 160 | XP 955820.1 | ND | ND | |
| | crassa | | | | | |
| 12 | Streptomyces | 161 | ASW31113.1 | + | + | |
| | fradiae | | | | | |
| 13 | Mycobacterium | 162 | AXN44890.1 | + | + | |
| | liflandii | | | | | |
| | 128FXT | | | | | |
| 14 | Mycobacteroides | 163 | CPX02987.1 | ND | ND | |
| | abscessus | | | | | |
| 15 | Mycolicibacterium | 164 | EJZ09834.1 | − | + | |
| | vaccae | | | | | |
| | ATCC 25954 | | | | | |
| 16 | Sorangium | 165 | KYF89500.1 | − | + | |
| | cellulosum | | | | | |
| 17 | Mycobacteroides | 166 | SLB41318.1 | − | − | − |
| | abscessus | | | | | |
| | subsp. | | | | | |
| | massiliense | | | | | |
| 18 | Curtobacterium | 167 | WP_028185036.1 | − | − | |
| | sp. | | | | | |
| | MCSS17_007 | | | | | |
| 19 | Nocardia | 168 | WP_029900022.1 | | | |
| | brasiliensis | | | | | |
| | ATCC 700358 | | | | | |
| 20 | Allokutzneria | 169 | WP_030430720.1 | − | + | |
| | albata | | | | | |
| 21 | Mycolicibacterium | 170 | WP_042264421.1 | + | − | |
| | vanbaalenii | | | | | |
| | PYR-1 | | | | | |
| 22 | Nocardia | 171 | KIA63422.1 | − | ++ | |
| | vulneris | | | | | |
| 23 | Mycolicibacterium | 172 | WP_045824606.1 | − | − | |
| | confluentis | | | | | |
| 24 | Mycolicibacter | 173 | WP_046295775.1 | − | ++ | |
| | arupensis | | | | | |
| 25 | Mycobacterium | 174 | WP_047542266.1 | + | + | |
| | simiae | | | | | |
| 26 | Streptomyces | 175 | WP_050362911.1 | + | + | |
| | sp. TSRI0395 | | | | | |
| 27 | Nocardia | 176 | WP_051178149.1 | − | + | |
| | seriolae | | | | | |
| 28 | Acaromyces | 177 | WP_051297098.1 | + | + | |
| | ingoldii | | | | | |
| 29 | Rhodococcus | 178 | WP_056069829.1 | + | + | |
| | sp. Leaf247 | | | | | |
| 30 | Streptomyces | 179 | WP_057605035.1 | − | − | |
| | sp. Root1310 | | | | | |
| 31 | Sphingobium | 180 | WP_062112979.1 | + | + | |
| | sp. MI1205 | | | | | |
| 32 | Streptomyces | 181 | WP_062207887.1 | − | − | |
| | yunnanensis | | | | | |
| 33 | Mycobacterium | 182 | WP_064915933.1 | − | − | |
| | sp. KMS | 1 | | | | |
| 34 | Mycobacterium | 183 | WP_064939119.1 | − | + | |
| | intracellulare | | | | | |
| | ATCC 13950 | | | | | |
| 35 | Mycolicibacterium | 184 | WP_067216488.1 | − | + | |
| | celeriflavum | | | | | |
| 36 | Mycolicibacterium | 185 | WP_067876059.1 | + | + | |
| | fallax | | | | | |
| 37 | Nocardia | 186 | WP_067585591.1 | − | − | |
| | terpenica | | | | | |
| 38 | Mycolicibacterium | 187 | WP_067973901.1 | − | ++ | |
| | peregrinum | | | | | |
| 39 | Mycobacterium | 188 | WP_068119938.1 | − | + | |
| | sp. E1386 | | | | | |
| 40 | Mycobacteroides | 189 | WP_052544110 | − | − | |
| | abscessus | | | | | |
| | subsp. | | | | | |
| | abscessus | | | | | |
| 41 | Mycobacterium | 190 | WP_073684817.1 | − | ++ | |
| | sp. SWH-M1 | | | | | |
| 42 | Mycobacteroides | 191 | WP_074248349.1 | − | +++ | |
| | abscessus | | | | | |
| | subsp. bolletii | | | | | |
| 43 | Curtobacterium | 192 | WP_071245982.1 | ND | ND | |
| | sp. | | | | | |
| | MCBA15_012 | | | | | |
| 44 | Mycobacteroides | 193 | WP_074331300.1 | | | |
| | abscessus | | | | | |
| | subsp. | | | | | |
| | abscessus | | | | | |
| 45 | Mycobacterium | 194 | WP_076102317.1 | − | − | |
| | colombiense | | | | | |
| 46 | Williamsia | 195 | WP_076478068.1 | + | + | |
| | sterculiae | | | | | |
| 47 | Mycobacteroides | 196 | WP_078287768.1 | − | − | |
| | chelonae | | | | | |
| 48 | Mycobacterium | 197 | WP_079921768.1 | − | − | |
| | sp. AT1 | | | | | |
| 49 | Brevibacterium | 198 | WP_082829042.1 | + | ++ | |
| | casei | | | | | |
| 50 | Mycobacterium | 199 | WP_083087901.1 | + | + | |
| | noviomagense | | | | | |
| 51 | Mycolicibacterium | 200 | OBB89769.1 | ++ | ++ | |
| | peregrinum | | | | | |
| 52 | Pseudonocardia | 201 | ODU29987.1 | − | − | |
| | sp. SCN 73-27 | | | | | |
| 53 | Chloroflexi | 202 | PKN69095.1 | − | + | |
| | bacterium | | | | | |
| | HGW- | | | | | |
| | Chloroflexi-5 | | | | | |
| 54 | Mycolicibacterium | 203 | WP_083123568.1 | + | ++ | |
| | tusciae | | | | | |
| 55 | Mycolicibacterium | 204 | WP_100516314.1 | + | ++ | |
| | goodii | | | | | |
| 56 | Frankia sp. | 205 | WP_007506938.1 | ++ | ++ | |
| | EUN1h | | | | | |
| 57 | Mycobacterium | 206 | WP_015306631.1 | | | |
| | sp. JS623 | | | | | |
| 58 | Legionella | 207 | WP_028384907.1 | | | |
| | moravica | | | | | |
| 59 | Pseudomonas | 208 | WP_101209428.1 | ++ | ++ | |
| | sp. 43NM1 | | | | | |
| 60 | Mycobacterium | 209 | WP_104865516.1 | ++ | ++ | |
| | sp. EPG1 | | | | | |
| 61 | Aphanothece cf. | 210 | WP_106220800.1 | + | − | |
| | minutissima | | | | | |
| | CCALA 015 | | | | | |
| 62 | Rhodococcus | 211 | WP_114723703.1 | − | + | |
| | sp. AG1013 | | | | | |
| 63 | Rhodococcus | 212 | WP_114723596.1 | + | + | |
| | sp. AG1013 | | | | | |
| 64 | Microbacterium | 213 | WP_116239091.1 | ++ | ++ | |
| | sp. AG157 | | | | | |
| 65 | Nocardia sp. | 214 | WP_120042211.1 | + | + | |
| | YIM PH 21724 | | | | | |
| 66 | Myceligenerans | 215 | WP_123814706.1 | + | + | |
| | xiligouense | | | | | |
| 67 | Pseudonocardiales | 216 | WP_132386047.1 | | | |
| | bacterium | | | | | |
| 68 | Streptomyces | 217 | WP_126639580.1 | − | − | |
| | scabrisporus | | | | | |
| 69 | Mycobacterium | 218 | WP_133895376.1 | − | + | |
| | [tuberculosis] | | | | | |
| | TKK-01-0051 | | | | | |
| 70 | Mycolicibacterium | 219 | WP_133759136.1 | + | ++ | |
| | llatzerense | | | | | |
| 71 | Rhodococcus | 220 | WP_094685926.1 | − | − | |
| | sp. 06-469-3-2 | | | | | |
| 72 | Gordonia sputi | 221 | WP_005206594.1 | | | |
| | NBRC 100414 | | | | | |
| 73 | Mycobacteroides | 222 | SHU01071.1 | ++ | ++ | |
| | abscessus | | | | | |
| | subsp. | | | | | |
| | abscessus | | | | | |
| 74 | Mycobacterium | 223 | PJE23808.1 | ++ | ++ | |
| | sp. | | | | | |
| 75 | Mycobacterium | 224 | WP_104061604.1 | ++ | ++ | |
| | decipiens | | | | | |
| 76 | Mycobacteroides | 225 | WP_100483059.1 | − | + | |
| | abscessus | | | | | |
| | subsp. Bolletii | | | | | |
| 77 | Pseudomonas | 226 | WP_095030548.1 | − | + | |
| | fragi | | | | | |
| 78 | Rhodococcus | 227 | WP_094713117.1 | − | + | |
| | sp. 15-1154-1 | | | | | |
| 79 | Rhodococcus | 228 | WP_037189966.1 | + | + | |
| | sp. 05-340-1 | | | | | |
| 80 | Mycobacterium | 229 | WP_087077450.1 | − | + | |
| | dioxanotrophicus | | | | | |
| 81 | Mycobacterium | 230 | ORV97129.1 | | | |
| | kubicae | | | | | |
| 82 | Mycobacterium | 231 | OBK63104.1 | − | − | |
| | colombiense | | | | | |
| 83 | Mycobacterium | 232 | WP_096287632.1 | | | |
| | ahvazicum | | | | | |
| 84 | Mycobacterium | 233 | WP_065073482.1 | − | + | |
| | colombiense | | | | | |
| 85 | Clavibacter | 234 | CAN00144.1 | | | |
| | michiganensis | | | | | |
| | subsp. | | | | | |
| | michiganensis | | | | | |
| | NCPPB 382 | | | | | |
| 86 | Mycobacteroides | 235 | WP_134080798.1 | + | − | |
| | abscessus | | | | | |
| | subsp. bolletii | | | | | |
| | 50594 | | | | | |
| 87 | Mycolicibacterium | 236 | EHB47859.1 | | | |
| | rhodesiae | | | | | |
| | JS60 | | | | | |
| 88 | Mycobacteroides | 237 | WP_064629783.1 | − | + | |
| | immunogenum | | | | | |
| 89 | Mycobacteroides | 238 | WP_070909839.1 | − | + | |
| | saopaulense | | | | | |
| 90 | Rhodococcus | 239 | WP_057479409.1 | − | − | |
| | sp. 06-412-2C | | | | | |
| 91 | Mycobacterium | 240 | WP_066859835.1 | − | − | |
| | sp. 1482292.6 | | | | | |
| 92 | Rhodococcus | 241 | AMY55468.1 | + | + | |
| | sp. PBTS 2 | | | | | |
| 93 | Mycolicibacterium | 242 | WP_058127019.1 | − | + | |
| | smegmatis | | | | | |
| 94 | Mycobacteroides | 243 | WP_070928686.1 | − | + | |
| | sp. H092 | | | | | |
| 95 | Mycobacterium | 244 | WP_064951073.1 | + | + | |
| | colombiense | | | | | |
| 96 | Mycobacterium | 245 | OBH69834.1 | − | − | |
| | sp. TKK-01-0059 | | | | | |
| 97 | Mycobacterium | 246 | SOX51546.1 | | | |
| | ahvazicum | | | | | |
| 98 | Mycobacterium | 247 | WP_085225350.1 | − | + | |
| | florentinum | | | | | |
| 99 | Mycobacterium | 248 | ORV62949.1 | | | |
| | europaeum | | | | | |
| 100 | Mycobacterium | 249 | OBH23094.1 | − | ++ | |
| | sp. E1319 | | | | | |
| 101 | Mycobacterium | 250 | ORA19103.1 | − | − | |
| | arosiense ATCC | | | | | |
| | BAA-1401 = | | | | | |
| | DSM 45069 | | | | | |
| 102 | Mycolicibacterium | 251 | AQT80557.1 | − | + | |
| | litorale | | | | | |
| 103 | Mycobacterium | 252 | WP_015304658.1 | | | |
| | sp. JS623 | | | | | |
| 104 | Candida | 253 | KGU19302.1 | − | − | |
| | albicans | | | | | |
| | SC5314 | | | | | |
| 105 | Mycobacterium | 254 | WP_015306631.1 | ++ | ++ | |
| | sp. JS623 | | | | | |
| 106 | Nocardia | 255 | AFU02004.1 | + | − | |
| | brasiliensis | | | | | |
| | ATCC 700358 | | | | | |
| 107 | Segniliparus | 256 | WP_007468889.1 | − | + | |
| | rugosus ATCC | | | | | |
| | BAA-974 | | | | | |
| 108 | Nocardia sp. | 257 | AHH98121.1 | | | |
| | Y48 | | | | | |
| 109 | Mycolicibacterium | 258 | WP_042264421.1 | + | − | |
| | vanbaalenii | | | | | |
| | PYR-1 | | | | | |
| 110 | Mycolicibacterium | 259 | WP_067876059.1 | + | + | |
| | fallax | | | | | |
| 111 | Brevibacteriumcasei | 260 | WP_082829042.1 | + | ++ | |
| 112 | Myceligenerans | 261 | WP_123814706.1 | + | + | |
| | xiligouense | | | | | |
| 113 | Mycolicibacterium | 262 | WP_133759136.1 | + | ++ | |
| | llatzerense | | | | | |
| 114 | Rhodococcus | 263 | AMY55468.1 | + | ++ | |
| | fascians D188 | | | | | |
| 115 | Mycobacteroides | 264 | WP_005082584.1 | | | |
| | abscessus | | | | | |
| | subsp. | | | | | |
| | abscessus | | | | | |

| Activity Legend | |
|---|---|
| | ΔF/min |
| − | no to little activity |
| + | active; 0.1-1 |
| ++ | active; 1-100 |
| +++ | active; 100-500 |
| ++++ | active; >500 |
| ND | Not Determined |

### Example 5. In Vivo Assays of CAR Homologs

To test the activity of CAR homologs in vivo, genes encoding selected CAR homologs were transformed into a strain of E. coli that also included introduced genes encoding 1) a 3-oxoadipyl-CoA thiolase (Thl), 2) a 3-oxoadipyl-CoA dehydrogenase (Hbd), 3) a 3-oxoadipyl-CoA dehydratratase (“crotonase” or Crt), 4) a 5-carboxy-2-pentenoyl-CoA reductases (Ter); 5) an aldehyde dehydrogenase (Ald), 6) a 6ACA transaminase (TA), and 7) a HMD-transaminase (TA2). The Thl, Hbd, Crt, Ter, Ald genes are reported in U.S. Pat. No. 8,377,680 (e. g., Example 8, which is incorporated by reference in its entirety). These genes were introduced in an E. coli strain that included all of the pathway enzymes necessary for producing HMD, with the exception of the CAR enzyme.

The vectors for expressing the CAR genes were transformed into the Thl/Hbd/Crt/Ter/Ald/TA/TA2 E. coli strain and transformants were tested for HMD production. The engineered E. coli cells were fed 2% glucose in minimal media, and after an 18 hour incubation at 35° C., the cells were harvested, and the supernatants were evaluated by analytical HPLC or standard LC/MS analytical method for HMD production. As shown in Table 8 and Table 9, expression of genes encoding the CAR enzymes in E. coli that included Thl, Hbd, Crt, Ter, Ald, TA, and TA2 genes resulted in HMD production by these strains.

### Example 6. CAR Variants

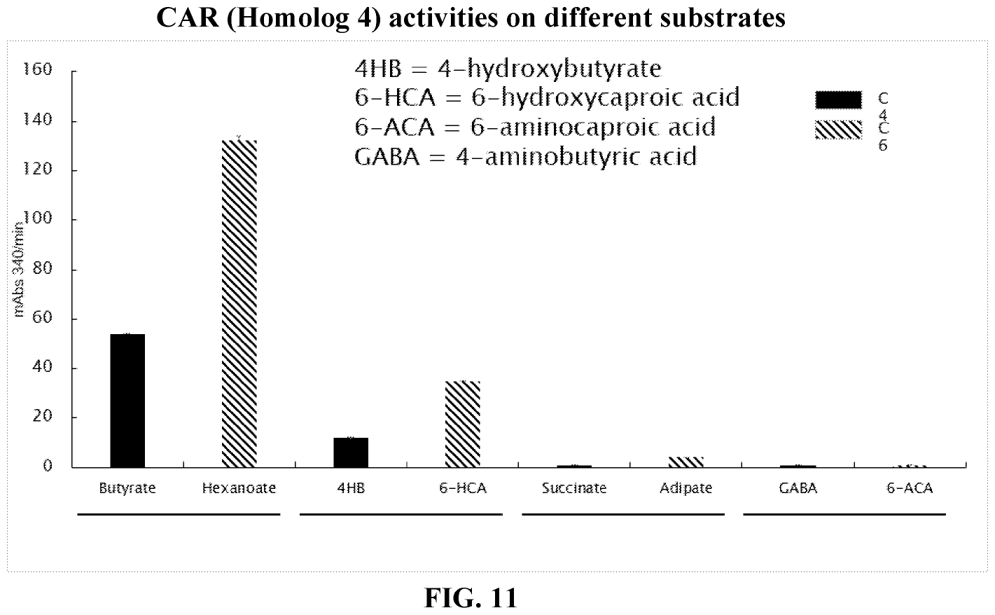

Amino acid positions were identified for mutations in CAR Homolog 4 (SEQ ID NO:153) and CAR Homolog 105 (SEQ ID NO:254) by examination of the crystal structure of the protein, and a gene encoding SEQ ID NOS: 153 and 254 was subjected to saturation mutagenesis at selected amino acid positions. The resulting variants were tested for activity in the lysate assay as described in Example 4. Results for the CAR SEQ ID NO:153 for eight different substrates are shown in FIG. 11.

Variants were generated by mutating the gene encoding the CAR enzyme (SEQ ID NOS:153 and 254) at amino acid positions for P141, L245, I247, W270, S274, K275, N276, F278, G279, N279insert, A282, A283, S299, I300, N335, S336, M389, G391, G414, G421, M422, F425, G636, D809, I810, L811, A812 and F929. Mutations were made singly and in combination with mutations at other amino acid positions. Mutations at the amino acid positions were made using degenerate primer sequences and PCR, where the altered gene sequence mixtures were transformed into E. coli. Transformants were tested in lysate assays as described in Example 4. Clones that provided lysates showing activity in the assays were retested in triplicate lysate assays. Clones that continued to demonstrate activity were then prepped for sequence analysis of the CAR genes they contained.

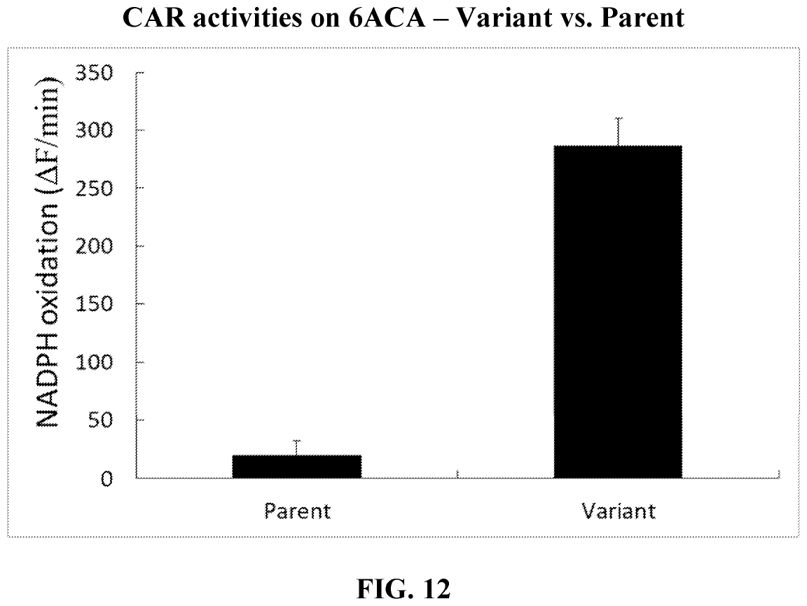

Table 9 and FIG. 12 provide the mutations found in the variant CAR gene sequences of the active clones. Variants demonstrating higher than wild type activity included single mutations and combinatorial (multiple) mutations in the CAR gene. Table 9 shows that multiple variants demonstrated greater activity than the wild type CAR (SEQ ID NO:152), with mutations at amino acid positions P141, L245, I247, W270, S274, K275, N276, F278, G279, N279insert, A282, A283, S299, I300, N335, S336, M389, G391, G414, G421, M422, F425, G636, D809, I810, L811, A812 and F929 (positions identified with respect to SEQ ID NO:152) resulting in multiple variants with higher activity than the wild type CAR from which they were derived. Table 9. CAR Variants

TABLE 9
CAR Variants
Variants of Homolog 4 (SEQ ID NO: 153)

| | | | In vivo HMD |
|---|---|---|---|
| Variant # | Mutations to Homolog 4 | Active 6ACA | production |
| | none | ++ | + |
| 1 | N335E | +++ | + |
| 2 | N335D | +++ | + |
| 3 | S274D | ++ | + |
| 4 | S274E | ++ | |
| 5 | K275D | ++ | |
| 6 | K275E | ++ | |
| 7 | S299D | ++++ | + |
| 8 | S299E | +++ | |
| 9 | M389D | ++++ | + |
| 10 | M389E | +++ | |
| 11 | G414D | +++ | + |
| 12 | G414E | +++ | + |
| 13 | G421D | ++ | |
| 14 | G421E | ++ | |
| 15 | M422D | +++ | + |
| 16 | M422E | ++ | |
| 17 | F425D | ++ | |
| 18 | F425E | ++ | |
| 19 | N335D A282P | +++ | |
| 20 | N335D A282V | +++ | |
| 21 | N335D A283C | ++++ | |
| 22 | N335D A283C F929L | +++ | |
| 23 | N335D A283C G636D | +++ | |
| 24 | N335D A283G | +++ | |
| 25 | N335D F278A | ++++ | |
| 26 | N335D F278C | +++ | |
| 27 | N335D F278S | ++++ | |
| 28 | N335D F278V | ++++ | |
| 29 | N335D G279V | +++ | |
| 30 | N335D I247M | ++++ | |
| 31 | N335D I247Q | +++ | |
| 32 | N335D I247T | ++++ | |
| 33 | N335D I247V | +++ | |
| 34 | N335D I300C | +++ | |
| 35 | N335D I300G | +++ | |
| 36 | N335D I300M | +++ | |
| 37 | N335D I300M | +++ | |
| 38 | N335D I300Y | +++ | |
| 39 | N335D K275A | +++ | |
| 40 | N335D K275D | +++ | |
| 41 | N335D K275D | +++ | |
| 42 | N335D K275E | +++ | |
| 43 | N335D K275M | +++ | |
| 44 | N335D K275N | ++++ | |
| 45 | N335D K275S | +++ | |
| 46 | N335D K275T | +++ | |
| 47 | N335D K275V | +++ | |
| 48 | N335D K275W | +++ | |
| 49 | N335D L245C | +++ | |
| 50 | N335D L245G | +++ | |
| 51 | N335D L245S | +++ | |
| 52 | N335D L245T | ++++ | |
| 53 | N335D L245V | ++++ | |
| 54 | N335D M389I | +++ | |
| 55 | N335D M389W | +++ | |
| 56 | N335D M422D | +++ | |
| 57 | N335D N276S | +++ | |
| 58 | N335D N279 INSERT | +++ | |
| 59 | N335D P141G | +++ | |
| 60 | N335D S299D | ++++ | |
| 61 | N335D S299E | +++ | |
| 62 | N335D S391G | +++ | |
| 63 | N335D W270M | +++ | |
| 64 | K275D N276S F278S A283C N335D | ++++ | |
| 65 | L245V K275D N276S F278S A283C | ++++ | |
| | N335D | | |
| 66 | I247M K275D N276S F278A A283C | ++++ | |
| | N335D | | |
| 67 | L245V K275T N276S F278S A283C | ++++ | |
| | N335D | | |
| 68 | I247M K275T N276S F278S A283C | ++++ | |
| | N335D | | |
| 69 | K275D N276S F278S A283C 1300G | ++++ | + |
| | N335D | | |
| 70 | L245T K275D N276S F278A A283C | ++++ | |
| | N335D | | |
| 71 | K275N F278S A283C S299D N335D | ++++ | |
| 72 | I247M K275N N276S F278S A283C | ++++ | |
| | N335D | | |
| 73 | K275N F278S A283C I300G N335D | ++++ | |
| 74 | K275N N276S F278S A283C I300G | ++++ | |
| | N335D | | |
| 75 | I247M K275T F278S A283C N335D | ++++ | |
| 76 | L245V K275D N276S F278A A283C | ++++ | |
| | N335D | | |
| 77 | K275N F278S A283C I300G N335D | ++++ | |
| 78 | K275N F278A A283C S299D | ++++ | |
| | N335D | | |
| 79 | K275N N276S F278A A283C | ++++ | |
| | N335D | | |
| 80 | L245V K275N N276S F278S A283C | ++++ | |
| | I300G N335D | | |
| 81 | L245T K275N N276S F278S A283C | ++++ | |
| | I300G N335D | | |
| 82 | K275N F278A S299D N335D | ++++ | |
| 83 | I247M K275N F278A A283C I300Y | ++++ | |
| | N335D | | |
| 84 | I247M K275N F278A A283C | ++++ | |
| | N335D | | |
| 85 | K275D N276S F278A N335D | ++++ | |
| 86 | K275T F278A A283C S299D N335D | ++++ | |
| 87 | F278A A282P N335D | ++++ | |
| 88 | F278A A283C N335D | ++++ | |
| 89 | K275N S299D N335D | ++++ | |
| 90 | L245T S299D N335D | ++++ | |
| 91 | K275D S299D N335D | ++++ | |
| 92 | K275N F278S N335D | ++++ | |
| 93 | S299D M389W N335D | ++++ | |
| 94 | G279V S299D N335D | ++++ | |
| 95 | F278A S283C N335D | ++++ | |
| 96 | K275D N276S N335D | ++++ | |
| 97 | G279V S299E N335D | ++++ | |
| 98 | I247M S299D N335D | ++++ | |
| 99 | P141G A282P N335D | ++++ | |
| 100 | L245T A282P N335D | ++++ | |
| 101 | F278S A283C N335D | ++++ | |
| 102 | K275D S284I S299D N335D | ++++ | |
| 103 | I247M I282P N335D | ++++ | |
| 104 | I247V K275D N276S F278S A283C | ++++ | |
| | I300G N335D | | |
| 105 | K275D S274C N276S F278S A283C | ++++ | |
| | I300G N335D | | |
| 106 | K275D S274A N276S F278S A283C | ++++ | |
| | I300G N335D | | |
| 107 | K275D S274P N276S F278S A283C | ++++ | |
| | I300G N335D | | |
| 108 | K275D N276S F278S A282F A283C | ++++ | |
| | I300G N335D | | |
| 109 | K275D N276S F278S A282P A283C | ++++ | |
| | I300G N335D | | |
| 110 | K275D N276S F278S A283C S299I | ++++ | |
| | I300G N335D | | |
| 111 | K275D N276S F278S A283C I300G | ++++ | |
| | N335D M389C | | |
| 112 | K275D N276S F278S A283C I300G | ++++ | |
| | N335D M389Y | | |
| 113 | K275D N276S F278S A283C I300G | ++++ | |
| | N335D M389S | | |

Variants of Homolog 105

| | | | In vivo HMD |
|---|---|---|---|
| Variant # | Mutations to Homolog 105 | Active 6ACA | production |
| | none | ++ | + |
| 114 | S336E | +++ | + |
| 115 | S336D | +++ | + |

Table 10 shows that combination of mutations at positions 245, 247, 274, 275, 276, 278, 282, 283, 299, 300 and 389 of homolog 4 can result in 165,888 total unique combinations. The combination mutants are in addition to N335D of homolog 4.

TABLE 10
Combination Mutants of CAR Variants
of Homolog 4 (SEQ ID NO: 153)

| Position # | Position | WT | Mutant | Total |
|---|---|---|---|---|
| 1 | 245 | L | T, V | 3 |
| 2 | 247 | I | M, T, V | 4 |
| 3 | 274 | S | C, A, P | 4 |
| 4 | 275 | K | D, N, T | 4 |
| 5 | 276 | N | S | 2 |
| 6 | 278 | F | S, A | 3 |
| 7 | 282 | A | F, P | 3 |
| 8 | 283 | A | C | 2 |
| 9 | 299 | S | D, I | 2 |
| 10 | 300 | I | G, Y | 3 |
| 11 | 389 | M | C, Y, S | 4 |
| Total unique combinations | | | | 165,888 |

### Example 7. Identification of TA2 Transaminases with Activity on 6-Aminocaproate Semialdehyde

Genes encoding TA2 transaminases were identified and tested as described in Example 1.

Table 11 shows that TA2 homologues having SEQ ID NOS:265 and 267-296 exhibited activity in converting 6-aminocaproate semialdehyde to HMND.

TABLE 11
TA2 Homologs

| | | SEQ | | | | | In vivo |
|---|---|---|---|---|---|---|---|
| Homolog | | ID | | Active | Active | Active | HMDA |
| # | Organism | NO: | Accession No. | 6ACA | GABA | HMD | Production |
| 1 | Escherichia | 265 | WP_001301395.1 | + | − | ++ | + |
| | coli | | | | | | |
| 2 | Candidatus | 266 | RTL46196.1 | − | − | − | |
| | melainabacteria | | | | | | |
| 3 | Enterobacteriaceae | 267 | WP_080350600.1 | + | + | ++ | |
| 4 | Citrobacter | 268 | WP_124021456.1 | ND | ND | ND | |
| 5 | Enterobacteriaceae | 269 | WP_126317364.1 | ND | ND | ND | |
| 6 | Chloroflexi | 270 | RIK54736.1 | + | + | ++ | |
| | bacterium | | | | | | |
| 7 | Anaerolineae | 271 | RCK76150.1 | + | + | ++ | |
| | bacterium | | | | | | |
| 8 | Citrobacter | 272 | WP_127674105.1 | − | + | + | |
| | freundii | | | | | | |
| 9 | Anaerovorax | 273 | WP_027400467.1 | + | + | + | |
| | odorimutans | | | | | | |
| 10 | Citrobacter | 274 | POT29884.1 | + | + | + | |
| | braakii | | | | | | |
| 11 | Anaerolineaceae | 275 | RJP50214.1 | ND | ND | ND | |
| | bacterium | | | | | | |
| 12 | Citrobacter | 276 | SUX95114.1 | + | − | + | |
| | koseri | | | | | | |
| 13 | Enterobacteriaceae | 277 | KGB03050.1 | − | + | + | |
| | bacterium | | | | | | |
| | ATCC 29904 | | | | | | |
| 14 | Lelliottia | 278 | ROR10477.1 | − | + | + | |
| | amnigena | | | | | | |
| 15 | Enterobacter | 279 | WP_014171705.1 | + | + | ++ | |
| 16 | Yokenella | 280 | WP_120816925.1 | − | + | − | |
| | regensburgei | | | | | | |
| 17 | Enterobacter | 281 | WP_119936919.1 | − | − | ++ | |
| | huaxiensis | | | | | | |
| 18 | Shigella | 282 | WP_128881201.1 | − | + | + | |
| | dysenteriae | | | | | | |
| 19 | Vibrio sp. | 283 | WP_124935623.1 | − | + | + | |
| | LJC006 | | | | | | |
| 20 | Desulfobulbaceae | 284 | RPH40555.1 | − | − | + | |
| | bacterium | | | | | | |
| 21 | Chloroflexi | 285 | RPI92554.1 | − | + | + | |
| | bacterium | | | | | | |
| 22 | Bacillus sp. | 286 | WP_120036151.1 | ND | ND | ND | |
| | PK3 68 | | | | | | |
| 23 | Thermosporothrix | 287 | WP_111320425.1 | + | + | + | |
| | hazakensis | | | | | | |
| 24 | Hafnia alvei | 288 | WP_111329186.1 | − | + | + | |
| 25 | Quasibacillus | 289 | WP_039231281.1 | − | + | ++ | |
| | thermotolerans | | | | | | |
| 26 | Lelliottia | 290 | WP_095283474.1 | + | + | + | |
| 27 | Aneurinibacillus | 291 | WP_091260983.1 | − | − | ++ | |
| | thermoaerophilus | | | | | | |
| 28 | Buttiauxella | 292 | WP_064555484.1 | − | + | ++ | |
| | noackiae | | | | | | |
| 29 | Desulfosarcina | 293 | WP_054695015.1 | + | + | + | |
| | cetonica | | | | | | |
| 30 | Leclercia | 294 | WP_082697316.1 | − | − | ++ | |
| | adecarboxylata | | | | | | |
| 31 | Neisseria | 295 | WP_047761121.1 | + | + | + | |
| | arctica | | | | | | |
| 32 | Citrobacter | 296 | WP_053088666.1 | − | + | ++ | |
| | freundii | | | | | | |

### Example 8. Screening of Aldehyde Dehydrogenases from Various Microbial Sources for Activity on Adipyl-CoA

Genes encoding aldehyde dehydrogenases (ALD) from various species were identified bioinformatically in the genomes of multiple species (Table 4). Genes encoding each of the aldehyde dehydrogenases were synthesized, expressed in E. coli, and evaluated for ALD activity.

The genes encoding the ALD enzyme candidates of Table 12 were cloned into a low-copy vector under a constitutive promoter and the constructs were transformed into E. coli using standard techniques. Transformants were cultured in LB medium in the presence of antibiotic overnight at 35° C., after which the cells were harvested at 15,000 rpm at room temperature. To prepare lysates, cells were resuspended in a chemical lysis solution containing lysozyme, nuclease, and 10 mM DTT and incubated at room temperature for at least 30 min. The resulting lysate was used to test aldehyde dehydrogenase activity.

The lysates (5 μl) were added to an assay mixture to result in a total volume of 20 μL with final concentrations of 0.1 M Tris-HCl, pH 7.5, 2.5 mM adipyl-CoA (AdCoA), and either 0.5 mM NADH or 0.5 mM NADPH.

This assay was used to screen ALD enzymes from various species. Some ALD candidates were also assayed using succinyl-CoA (SuCoA) or acetyl-CoA (AcCoA) as substrates. AdCoA, SuCoA, and AcCoA were obtained from commercial suppliers. Activity was monitored by a linear decrease in fluorescence of NADH or NADPH in the presence of the CoA substrate. ALDs that were significantly active on adipyl-CoA using either the NADH or NADPH were designated as positive (+) in Table 4 and those with little to no activity were designated with a minus (−).

What is claimed is:

1. An engineered carboxylic acid reductase (CAR) enzyme comprising at least one alteration of an amino acid of SEQ ID NOS:152, 153 or 254.

2. The engineered CAR of claim 1, wherein the engineered CAR enzyme is capable of:
(a) forming 6-aminocaproate semialdehyde from a 6-aminocaproic acid substrate;
(b) forming 6-aminocaproate semialdehyde from a 6-aminocaproic acid substrate at a greater rate as compared to the wild type CAR;
(c) having a higher affinity for a 6-aminocaproic acid substrate as compared to the wild type CAR, or
(d) any combination of (a), (b) and (c).

3. The engineered CAR of any one of claims 1-2, wherein the engineered CAR comprises one or more amino acid alterations at one or more residue positions selected from the group consisting of P141, L245, I247, W270, S274, K275, N276, F278, G279, N279insert, A282, A283, S299, I300, N335, S336, M389, G391, G414, G421, M422, F425, G636, D809, I810, L811, A812 and F929 or a combination thereof of SEQ ID NO:152, 153 or 254.

4. The engineered CAR of any one of claims 1-3, wherein the engineered CAR comprises one or more amino acid alterations at one or more residue positions selected from the group consisting of P141, L245, I247, W270, S274, K275, N276, F278, G279, N279insert, A282, A283, S299, I300, N335, S336, M389, G391, G414, G421, M422, F425, G636, D809, I810, L811, A812 and F929 or a combination thereof of SEQ ID NO:152, 153 or SEQ ID NO:254 that is a conservative amino acid substitution.

5. The engineered CAR of any one of claims 1-3, wherein the engineered CAR comprises one or more amino acid alterations at one or more residue positions selected from the group consisting of P141, L245, I247, W270, S274, K275, N276, F278, G279, N279insert, A282, A283, S299, I300, N335, S336, M389, G391, G414, G421, M422, F425, G636, D809, I810, L811, A812 and F929 or a combination thereof of SEQ ID NO:152, 153 or 254 that is a non-conservative amino acid substitution.

6. The engineered CAR of any one of claim 1-5, wherein the at least one amino acid alteration of the engineered protein is an alteration selected from Table 9 and combinations thereof of SEQ ID NO:152, 153 or 254.

7. The engineered CAR of any one of claims 1-6, wherein the amino acid alteration comprises at least two, three, four, five, six, seven, eight, or more of an amino acid sequence of SEQ ID NO:152, 153 or 254.

8. The engineered CAR of any one of claims 1-7, wherein the amino acid positions comprises at least two, three, four, five, six, seven, eight, or more amino acid positions of SEQ ID NO:152, 153 or 254.

9. The engineered CAR of any one of claims 1-8, wherein the engineered CAR comprises an activity that is at least 20% higher than the activity of the CAR of SEQ ID NO:152, 153 or 254.

10. The engineered CAR of any one of claims 1-9, wherein the engineered CAR comprises one or more amino acid alterations selected from the group consisting of: N335E; N335D; S274D; S274E; K275D; K275E; S299D; S299E; M389D; M389E; G414D; G414E; G421D; G421E; M422D; M422E; F425D; F425E; N335D and A282P; N335D and A282V; N335D and A283C; N335D, A283C and F929L; N335D, A283C and G636D; N335D and A283G; N335D and F278A; N335D and F278C; N335D and F278S; N335D and F278V; N335D and G279V; N335D and I247M; N335D and I247Q; N335D and 1247T; N335D and 1247V; N335D and I300C; N335D and I300G; N335D and I300M; N335D and I300M; N335D and I300Y; N335D and K275A; N335D and K275D; N335D and K275D; N335D and K275E; N335D and K275M; N335D and K275N; N335D and K275S; N335D and K275T; N335D and K275V; N335D and K275W; N335D and L245C; N335D and L245G; N335D and L245S; N335D and L245T; N335D and L245V; N335D and M389I; N335D and M389W; N335D and M422D; N335D and N276S; N335D and N279INSERT; N335D and P141G; N335D and S299D; N335D and S299E; N335D and S391G; N335D and W270M; K275D, N276S, F278S, A283C and N335D; L245V, K275D, N276S, F278S, A283C and N335D; I247M, K275D, N276S, F278A, A283C and N335D; L245V, K275T, N276S, F278S, A283C and N335D; I247M, K275T, N276S, F278S, A283C and N335D; K275D, N276S, F278S, A283C, I300G and N335D; L245T, K275D, N276S, F278A, A283C and N335D; K275N, F278S, A283C, S299D and N335D; I247M, K275N, N276S, F278S, A283C and N335D; K275N, F278S, A283C, I300G and N335D; K275N, N276S, F278S, A283C, I300G and N335D; I247M, K275T, F278S, A283C and N335D; L245V, K275D, N276S, F278A, A283C and N335D; K275N, F278S, A283C, I300G and N335D; K275N, F278A, A283C, S299D and N335D; K275N, N276S, F278A, A283C and N335D; L245V, K275N, N276S, F278S, A283C, I300G and N335D; L245T, K275N, N276S, F278S, A283C, I300G and N335D; K275N, F278A, S299D and N335D; I247M, K275N, F278A, A283C, I300Y and N335D; I247M, K275N, F278A, A283C and N335D; K275D, N276S, F278A and N335D; K275T, F278A, A283C, S299D and N335D; F278A, A282P and N335D; F278A, A283C and N335D; K275N, S299D and N335D; L245T, S299D and N335D; K275D, S299D and N335D; K275N, F278S and N335D; S299D, M389W and N335D; G279V, S299D and N335D; F278A, S283C and N335D; K275D, N276S and N335D; G279V, S299E and N335D; I247M, S299D and N335D; P141G, A282P and N335D; L245T, A282P and N335D; F278S, A283C and N335D; K275D, S284I, S299D and N335D; I247M, I282P and N335D; I247V, K275D, N276S, F278S, A283C, I300G and N335D; I247V, K275D, N276S, F278S, A283C, I300G and N335D; K275D, S274A, N276S, F278S, A283C, I300G and N335D; K275D, S274P, N276S, F278S, A283C, I300G and N335D; K275D, N276S, F278S, A282F, A283C, I300G and N335D; K275D, N276S, F278S, A282P, A283C, I300G and N335D; K275D, N276S, F278S, A283C, S299I, I300G and N335D; K275D, N276S, F278S, A283C, I300G, N335D and M389C; K275D, N276S, F278S, A283C, I300G, N335D and M389Y; and K275D, N276S, F278S, A283C, I300G, N335D and M389S.

11. The engineered CAR of any one of claims 1-10, wherein the engineered CAR comprises at least one alteration of an amino acid of SEQ ID NO: 153, and wherein the engineered CAR comprises one or more amino acid alterations selected from the group consisting of: K275D, N276S, F278S, A283C, I300G and N335D; I247V, K275D, N276S, F278S, A283C, I300G and N335D; I247V, K275D, N276S, F278S, A283C, I300G and N335D; K275D, S274A, N276S, F278S, A283C, I300G and N335D; K275D, S274P, N276S, F278S, A283C, I300G and N335D; K275D, N276S, F278S, A282F, A283C, I300G and N335D; K275D, N276S, F278S, A282P, A283C, I300G and N335D; K275D, N276S, F278S, A283C, S299I, I300G and N335D; K275D, N276S, F278S, A283C, I300G, N335D and M389C; K275D, N276S, F278S, A283C, I300G, N335D and M389Y; and K275D, N276S, F278S, A283C, I300G, N335D and M389S.

12. A nucleic acid encoding the engineered CAR of any one of claims 1-11.

13. The nucleic acid of claim 12, wherein the nucleic acid sequence encoding the engineered CAR is operatively linked to a promoter.

14. A vector comprising the nucleic acid of claim 13.

15. A non-naturally occurring microbial organism comprising an exogenous nucleic acid encoding:
(a) an engineered CAR selected from any one of claims 1-11;
(b) a CAR comprising an amino acid sequence having at least 50% sequence identity to at least 25, 50, 75, 100, 150, 200, 250, 300, or more contiguous amino acids of any of SEQ ID NOS:150-165, 168-171, 173-178, 180, 183-185, 187-188, 190-193, 195, 198-200, 202-216, 218-219, 221-230, 232-238, 241-244, 246-249, 251-252 and 255-264; or
(c) a hexamethylenediamine (HMD) transaminase (TA2) enzyme having at least 50% sequence identity to at least 25, 50, 75, 100, 150, 200, 250, 300, or more contiguous amino acids of any of SEQ ID NOS:265 and 267-296.

15. The non-naturally occurring microbial organism of claim 15, further comprising an exogenous nucleic acid encoding:
(a) an engineered transaminase (TA) enzyme comprising at least one alteration of an amino acid of SEQ ID NOS:1, 13 or 31;
(b) an engineered transaminase (TA) enzyme comprising one or more amino acid alterations at one or more positions selected from residues V114, S136, T148, P153, I203, I204, P206, V207, V111, T216, A237, T264, M265, L386, G19, C22, D70, R94, D99, T109, E112, A113, F137, G144, I149, K150, Y154, S178, L186, Q208, L234, T242, A315, K318, R338, G336, L386, V390, A406, S416, A421, G17, M21, A50, A76, Y77, Q78, 179, G84, F107, T108, K119, G139, M142, A152, P153, E205, G209, G211, D238, M285, A290, G291, G292, L293, Y297, M353, S387, S388, and G392 or a combination thereof of SEQ ID NO:1;
(c) an engineered transaminase (TA) enzyme comprising at least one amino acid alteration of the engineered protein selected from an alteration set forth in Table 7 and combinations thereof of SEQ ID NO:1, or
(d) a transaminase comprising an amino acid sequence having at least 50% sequence identity to at least 25 or more contiguous amino acids of any of SEQ ID NO: 1, 3, 4, 5, 9, 12, 13, 26, 27, 30, 31, 38, 50, 52, 64, 74, 78, 79, 81, 91, 106, 108, and 116.

17. The engineered TA of claim 16, wherein the one or more amino acid alterations at one or more residue positions or a combination thereof of SEQ ID NO:1 is selected from a conservative or a non-conservative amino acid alteration.

18. The non-naturally occurring microbial organism of any one of claims 15-17, wherein the exogenous nucleic acid is heterologous.

19. The non-naturally occurring microbial organism of any one of claims 15-17, wherein the exogenous nucleic acid is homologous.

20. The non-naturally occurring microbial organism of any one of claims 15-19, comprising a CAR having an amino acid sequence selected from the group consisting of SEQ ID NOS:150-165, 168-171, 173-178, 180, 183-185, 187-188, 190-193, 195, 198-200, 202-216, 218-219, 221-230, 232-238, 241-244, 246-249, 251-252 and 255-264.

21. The non-naturally occurring microbial organism of any one of claims 15-19, comprising a CAR having an amino acid sequence selected from the group consisting of CAR variants 1-115 set forth in Table 9.

22. The non-naturally occurring microbial organism of any one of claims 15-21, comprising a HMD transaminase (TA2) having an amino acid sequence selected from the group consisting of SEQ ID NOS:265, and 267-296.

23. The non-naturally occurring microbial organism of any one of claims 16-22, comprising an engineered transaminase (TA) having an amino acid sequence selected from the group consisting of TA variants 1-233 set forth in Table 7.

24. The non-naturally occurring microbial organism of any one of claims 15-23, further comprising at least one exogenous nucleic acid encoding an aldehyde dehydrogenase (ALD) enzyme that reacts with adipyl-CoA to form adipate-semialdehyde.

25. The non-naturally occurring microbial organism of claim 24, comprising an ALD enzyme having an amino acid sequence selected from the group consisting of SEQ ID NOS:141-143 and 297-370.

26. The non-naturally occurring microbial organism of any one of claims 24-25, wherein the ALD enzyme has greater catalytic efficiency for adipyl-CoA substrate as compared to succinyl-CoA, acetyl-CoA, or both succinyl-CoA and acetyl-CoA substrates, and/or the aldehyde dehydrogenase has higher turnover number for adipyl-CoA substrate as compared to succinyl-CoA, acetyl-CoA, or both succinyl-CoA and acetyl-CoA substrates.

27. The non-naturally occurring microbial organism of any one of claims 25-26, comprising a hexamethylenediamine (HMD) pathway having a HMD pathway enzyme expressed in sufficient amounts to produce HMD, wherein said HMD pathway comprises (1) 3-oxoadipyl-CoA thiolase, (2) hydroxyadipyl-CoA dehydrogenase (HBD), (3) crotonase, (4) trans-enoylCoA reductase (TER), (5) 6ACA-aldehyde dehydrogenase (6ACA-ALD), (6) 6ACA-transaminase (TA), (7) carboxylic acid reductase (CAR), and (8) HMD-transaminase (TA2).

28. The non-naturally occurring microbial organism of claim 27, further comprising an exogenous nucleic acid encoding a phosphopantetheinyl transferase HMD pathway enzyme.

29. The non-naturally occurring microbial organism of any one of claims 27-28, wherein the microbial organism comprises one, two, three, four, five, six, seven, eight or nine exogenous nucleic acids each encoding a HMD pathway enzyme.

30. The non-naturally occurring microbial organism of any one of claims 27-28, wherein the microbial organism comprises exogenous nucleic acids encoding each of the enzymes of the HMD pathway.

31. The non-naturally occurring microbial organism of any one of claims 27-30, wherein the at least one exogenous nucleic acid is a heterologous nucleic acid.

32. The non-naturally occurring microbial organism of any one of claims 27-31, wherein the non-naturally occurring microbial organism is in a substantially anaerobic culture medium.

33. The non-naturally occurring microbial organism of any one of claims 27-32, wherein the microbial organism is a species of bacteria, yeast, or fungus.

34. The non-naturally occurring microbial organism of any one of claims 27-33, wherein the non-naturally occurring microbial organism is capable of producing at least 10% more 6-aminocaproate semialdehyde, HMD or both compared to a control microbial organism that does not comprise the exogenous nucleic acid of any one of claims 15-26.

35. The non-naturally occurring microbial organism of any one of claims 27-34, wherein the non-naturally occurring microbial organism converts more:
(a) adipate semialdehyde to 6-aminocaproic acid;
(b) 6-aminocaproic acid to 6-aminocaproate semialdehyde, and/or
(c) 6-aminocaproate semialdehyde to HMD,
compared to a control microbial organism substantially identical to the non-naturally occurring microbial organism, with the exception that the control microbial organism does not comprise the exogenous nucleic acid of any one of claims 15-23.

36. A non-naturally occurring microbial organism comprising an exogenous nucleic acid encoding:
(a) an engineered CAR selected from any one of claims 1-11, or
(b) a CAR comprising an amino acid sequence having at least 50% sequence identity to at least 25 or more contiguous amino acids of any of SEQ ID NO: 150-165, 168-171, 173-178, 180, 183-185, 187-188, 190-193, 195, 198-200, 202-216, 218-219, 221-230, 232-238, 241-244, 246-249, 251-252 and 255-264.

37. The non-naturally occurring microbial organism of claim 36, further comprising an exogenous nucleic acid encoding:
(a) an engineered transaminase (TA) enzyme comprising at least one alteration of an amino acid of SEQ ID NOS:1, 13 or 31;
(b) an engineered transaminase (TA) enzyme comprising one or more amino acid alterations at one or more positions selected from residues V114, S136, T148, P153, I203, I204, P206, V207, V111, T216, A237, T264, M265, L386, G19, C22, D70, R94, D99, T109, E112, A113, F137, G144, I149, K150, Y154, S178, L186, Q208, L234, T242, A315, K318, R338, G336, L386, V390, A406, S416, A421, G17, M21, A50, A76, Y77, Q78, 179, G84, F107, T108, K119, G139, M142, A152, P153, E205, G209, G211, D238, M285, A290, G291, G292, L293, Y297, M353, S387, S388, and G392 or a combination thereof of SEQ ID NO:1;
(c) an engineered transaminase (TA) enzyme comprising at least one amino acid alteration of the engineered protein selected from an alteration set forth in Table 7 and combinations thereof of SEQ ID NO:1, or
(d) a transaminase comprising an amino acid sequence having at least 50% sequence identity to at least 25 or more contiguous amino acids of any of SEQ ID NO: 1, 3, 4, 5, 9, 12, 13, 26, 27, 30, 31, 38, 50, 52, 64, 74, 78, 79, 81, 91, 106, 108, and 116.

38. The engineered TA of claim 37, wherein the one or more amino acid alterations at one or more residue positions or a combination thereof of SEQ ID NO:1 is selected from a conservative or a non-conservative amino acid alteration.

39. The non-naturally occurring microbial organism of any one of claims 36-38, wherein the exogenous nucleic acid is heterologous.

40. The non-naturally occurring microbial organism of any one of claims 36-38, wherein the exogenous nucleic acid is homologous.

41. The non-naturally occurring microbial organism of any one of claims 36-40, comprising a CAR having an amino acid sequence selected from the group consisting of SEQ ID NOS:150-165, 168-171, 173-178, 180, 183-185, 187-188, 190-193, 195, 198-200, 202-216, 218-219, 221-230, 232-238, 241-244, 246-249, 251-252 and 255-264.

42. The non-naturally occurring microbial organism of any one of claims 36-40, comprising a CAR having an amino acid sequence selected from the group consisting of CAR variants 1-115 set forth in Table 9.

43. The non-naturally occurring microbial organism of any one of claims 37-42, comprising an engineered transaminase (TA) having an amino acid sequence selected from the group consisting of TA variants 1-233 set forth in Table 7.

44. The non-naturally occurring microbial organism of any one of claims 36-43, comprising a 1,6-hexanediol (HDO) pathway having a HDO pathway enzyme expressed in sufficient amounts to produce HDO, wherein said HDO pathway comprises (1) thiolase, (2) hydroxyadipyl-CoA dehydrogenase (HBD), (3) crotonase, (4) trans-enoylCoA reductase (TER), (5) 6ACA-aldehyde dehydrogenase (6ACA-ALD), (6) 6ACA-transaminase (TA), (7) carboxylic acid reductase (CAR), (8) 6-aminocaproate semialdehyde reductase, (9) 6-aminohexanol aminotransferase or oxidoreductase, and (10) 6-hydroxyhexanal reductase.

45. The non-naturally occurring microbial organism of claim 44, further comprising an exogenous nucleic acid encoding a phosphopantetheinyl transferase HDO pathway enzyme.

46. The non-naturally occurring microbial organism of any one of claims 44-45, wherein the microbial organism comprises one, two, three, four, five, six, seven, eight, nine, ten or eleven exogenous nucleic acids each encoding a HDO pathway enzyme.

47. The non-naturally occurring microbial organism of any one of claims 44-46, wherein the microbial organism comprises exogenous nucleic acids encoding each of the enzymes of the HDO pathway.

48. The non-naturally occurring microbial organism of any one of claims 44-47, wherein the at least one exogenous nucleic acid is a heterologous nucleic acid.

49. The non-naturally occurring microbial organism of any one of claims 44-48, wherein the non-naturally occurring microbial organism is in a substantially anaerobic culture medium.

50. The non-naturally occurring microbial organism of any one of claims 44-49, wherein the microbial organism is a species of bacteria, yeast, or fungus.

51. The non-naturally occurring microbial organism of any one of claims 44-50, wherein the non-naturally occurring microbial organism is capable of producing at least 10% more 6-aminocaproate semialdehyde, HDO or both compared to a control microbial organism that does not comprise the exogenous nucleic acid of any one of claims 36-43.

52. The non-naturally occurring microbial organism of any one of claims 44-51, wherein the non-naturally occurring microbial organism converts more:
(a) adipate semialdehyde to 6-aminocaproic acid, and/or
(b) 6-aminocaproic acid to 6-aminocaproate semialdehyde.
compared to a control microbial organism substantially identical to the non-naturally occurring microbial organism, with the exception that the control microbial organism does not comprise the exogenous nucleic acid of any one of claims 36-43.

53. A method for producing hexamethylenediamine (HMD), comprising culturing the non-naturally occurring microbial organism of any one of claims 27-35 under conditions and for a sufficient period of time to produce HMD.

54. The method of claim 53, wherein said method further comprises separating the HMD from other components in the culture.

55. The method of claim 54, wherein the separating comprises extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

56. A method for producing 1,6-hexanediol (HDO), comprising culturing the non-naturally occurring microbial organism of any one of claims 43-52 under conditions and for a sufficient period of time to produce HMD.

57. The method of claim 56, wherein said method further comprises separating the HMD from other components in the culture.

58. The method of claim 57, wherein the separating comprises extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

59. A culture medium comprising bioderived HMD, 6-aminocaproate semialdehyde, and/or HDO, wherein said bioderived HMD, 6-aminocaproate semialdehyde, and/or HDO has a carbon-12, carbon-13 and carbon-14 isotope ratio that reflects an atmospheric carbon dioxide uptake source.

60. The culture medium of claim 59, wherein the bioderived HMD, 6-aminocaproate semialdehyde, and/or HDO is produced by:
(a) the non-naturally occurring microbial organism of any one of claims 15-35;
(b) the non-naturally occurring microbial organism of any one of claims 36-52;
(c) the method of any one of claims 53-55; or
(d) the method of any one of claims 56-58.

61. The culture medium of any one of claims 59 and 61, wherein the culture medium comprises:
(a) the engineered CAR of any one of claims 1-11;
(b) the nucleic acid of any one of claims 12-14;
(c) the engineered transaminase (TA) enzyme of any one of claims 16-17, 37-38, and 43;
(d) the nucleic acid encoding the TA enzyme of any one of claims 16-17, 37-38, and 43;
(e) the engineered hexamethylenediamine (HMD) transaminase (TA2) enzyme of claim 15;
(f) the nucleic acid encoding the TA2 enzyme of claim 15;
(g) the aldehyde dehydrogenase (ALD) enzyme of any one of claims 24-27;
(h) the nucleic acid encoding the ALD enzyme of any one of claims 24-27;
(i) the non-naturally occurring microbial organism of any one of claims 15-35; or
(j) the non-naturally occurring microbial organism of any one of claims 36-52.

62. The culture medium of any one of claims 59-61, wherein said culture medium is separated from a non-naturally occurring microbial organism that produces bioderived HMD, 6-aminocaproate semialdehyde, and/or HDO.

63. Bioderived HMD, 6-aminocaproate semialdehyde, and/or HDO having a carbon-12, carbon-13 and carbon-14 isotope ratio that reflects an atmospheric carbon dioxide uptake source.

64. The bioderived HMD, 6-aminocaproate semialdehyde, and/or HDO of claim 63 is produced by:
(a) the non-naturally occurring microbial organism of any one of claims 15-35;
(b) the non-naturally occurring microbial organism of any one of claims 36-52;
(c) the method of any one of claims 53-55; or
(d) the method of any one of claims 56-58.

65. The bioderived HMD, 6-aminocaproate semialdehyde, and/or HDO of any one of claims 63-64, wherein said bioderived HMD, 6-aminocaproate semialdehyde, and/or HDO has an Fm value of at least 80%, at least 85%, at least 90%, at least 95% or at least 98%.

66. A composition comprising the bioderived HMD, 6-aminocaproate semialdehyde, and/or HDO of any one of claims 63-65, and a compound other than the bioderived HMD, 6-aminocaproate semialdehyde, and/or HDO.

67. The composition comprising the bioderived HMD, 6-aminocaproate semialdehyde, and/or HDO of claim H3, wherein the composition further comprises a portion of the non-naturally occurring microbial organism of any one of claims 15-42.

68. The composition comprising the bioderived HMD, 6-aminocaproate semialdehyde, and/or HDO of any one of claims 66-67, or a cell lysate or culture supernatant thereof.
